(19) United States
(12) Patent Application Publication
(10) Pub. No.: US 2026/0144821 A1
(43) Pub. Date: May 28, 2026

(54) Treatment of Blood Transfusion-Dependent Patients with Hematologic Malignancies

(71) Applicant: Hemanext Inc., Lexington, MA (US)

(72) Inventors: Martin CANNON, Chevy Chase, MD (US); Andrew DUNHAM, Tower Lakes, IL (US); Laurel OMERT, Malvern, PA (US); Kimberly DORSCH, Chesapeake, VA (US); Michael DIOGUARDI, Medford, MA (US)

(73) Assignee: Hemanext Inc., Lexington, MA (US)

(21) Appl. No.: 19/121,567

(22) PCT Filed: Oct. 20, 2023
(86) PCT No.: PCT/US2023/077484

§ 371 Date: Apr. 16, 2025

Related U.S. Application Data
(60) Provisional application No. 63/418,024, filed on Oct. 20, 2022.
(60) Provisional application No. 63/484,693, filed on Feb. 13, 2023.
(60) Provisional application No. 63/591,597, filed on Oct. 19, 2023.

(51) Int. Cl. A61K 35/18; A61K 47/02; A61K 47/14; A61K 47/26; A61P 7/00

(52) U.S. Cl. CPC A61K 35/18; A61K 47/02; A61K 47/14; A61K 47/26; A61P 7/00

(57) ABSTRACT

The present disclosure relates to methods for treating blood transfusion-dependent hematological malignancy patients with oxygen reduced blood.

### CROSS-REFERENCE TO RELATED APPLICATIONS

This application claims the benefit of U.S. Provisional Application No. 63/418,024, filed Oct. 20, 2022, U.S. Provisional Application No. 63/484,693, filed Feb. 13, 2023, and U.S. Provisional Application No. 63/591,597, filed Oct. 19, 2023, all of which are incorporated by reference in their entireties herein.

### FIELD OF THE INVENTION

The present disclosure relates to treatment of blood transfusion-dependent hematological malignancy patients, including patients having myelodysplastic syndromes (MDS), with oxygen and carbon dioxide reduced blood.

### BACKGROUND OF THE INVENTION

Hematologic malignancies such as myelodysplastic syndromes (MDS), multiple myeloma, and leukemia are increasing in prevalence in Norway as the population average age continues to increase. Collectively, hematologic malignancies refer to various disorders of the bone marrow which result in abnormal blood cell production. Leukemia consists of several malignant disorders that present with increased numbers of leucocytes in the blood and bone marrow. Multiple myeloma is characterized by uncontrolled growth of plasma cells in the bone marrow. MDS consist of bone marrow diseases associated with ineffective hematopoiesis, resulting in morphologically abnormal blood cells. See Brigle et al., “Pathobiology and Diagnosis of Multiple Myeloma,” Semin Oncol Nurs 33(3):225-236 (2017).

In hematologic malignancy cases, anemia can result from the overcrowding or ineffective production of normal blood cells, leading to inadequate oxygen delivery. Patients with hematologic malignancies often require regular blood transfusions at some time during their treatment to alleviate the symptoms of severe anemia, which can include fatigue, weakness, and exacerbation of other underlying medical conditions. While treatment varies with disease severity, transfusions are given at approximately weekly intervals in intensive treatment. See Hasserjian, et al., “Syndrome Updated,” Pathobiology 86(1):7-13 (2019). In relying on stored donor RBCs to address anemia, the goal is not to rapidly rebuild a normal red cell mass, but to minimize morbidity and mortality with the lowest possible exposure. See Cannas et al., “Supportive care in patients with acute leukemia: historical perspectives,” Blood Transfus 13(2):205-220 (2015). No consensus exists on the appropriate time to commence transfusions or on the correct transfusion threshold for optimal care. Some studies show reduced survival with increased transfusion density. See Cserti-Gazdewich, “Shifting ground and gaps in transfusion support of patients with hematological malignancies,” Hematology Am Soc Hematol Educ Program 2018 (1):553-560 (2018). Iron overload, a consequence of transfusion dependency, is also independently associated with poor outcomes for patients with MDS. See de Swart et al., “Impact of red blood cell transfusion dose density on progression-free survival in patients with lower-risk myelodysplastic syndromes,” Haematologica 105(3):632-639 (2020); and Sanz et al., “Independent Impact of Iron Overload and Transfusion Dependency on Survival and Leukemic Evolution in Patients with Myelodysplastic Syndrome,” Blood 112(11):640 (2008). Iron overload with organ impairment may be fatal in those who are heavily iron-overloaded. See Malcovati et al., “Time-dependent prognostic scoring system for predicting survival and leukemic evolution in myelodysplastic syndromes.” J Clin Oncol 25(23):3503-3510 (2007). Therefore, where a patient has no fatigue-induced limitation on quality of life or function at home, a more conservative approach may be preferred to avoid potential adverse effects and improve outcomes. See Gao et al., “Clinical outcomes of transfusion-associated iron overload in patients with refractory chronic anemia,” Patient Prefer Adherence 8:513-517 (2014).

Here, we demonstrate for the first time that oxygen and carbon dioxide reduced, leukoreduced blood manufactured using a Hemanext ONE system may decrease the required number of RBC transfusions, increase the time between transfusions for transfusion-dependent patients, reduce the risk and occurrence of transfusion-related iron overload, and reduce adverse events in patients with hematological malignancies.

### SUMMARY

The present specification addresses the need to develop effective treatments for blood transfusion-dependent hematological malignancy patients, including patients having myelodysplastic syndromes (MDS).

The present disclosure provides for, and includes, a method for reducing the blood transfusion volume for a hematologic malignancy patient in need thereof comprising administering at least two units of oxygen and carbon dioxide reduced, leukoreduced red blood cells to the hematologic malignancy patient in need thereof, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, leukoreduced red blood cells are administered to the hematologic malignancy patient in need thereof in a single transfusion event, and wherein the average volume of subsequent blood transfusions administered to the hematologic malignancy patient in need thereof after the administering of the at least two units of oxygen and carbon dioxide reduced, leukoreduced red blood cells is reduced by at least 10%.

The present disclosure provides for, and includes, a method for increasing the blood transfusion interval for a hematologic malignancy patient in need thereof comprising administering at least two units of oxygen and carbon dioxide reduced, leukoreduced red blood cells to the hematologic malignancy patient in need thereof, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, leukoreduced red blood cells are administered to the hematologic malignancy patient in need thereof in a single transfusion event, and wherein the average interval between subsequent blood transfusions administered to the hematologic malignancy patient in need thereof after the administering of the at least two units of oxygen and carbon dioxide reduced, leukoreduced red blood cells is increased by at least 10%.

The present disclosure provides for, and includes, a method for increasing the hemoglobin increment of a hematologic malignancy patient in need thereof comprising administering at least two units of oxygen and carbon dioxide reduced, leukoreduced red blood cells to the hematologic malignancy patient in need thereof, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, leukoreduced red blood cells are administered to the hematologic malignancy patient in need thereof in a single transfusion event, and wherein the hemoglobin increment of the hematologic malignancy patient in need thereof is increased by at least 1.0 grams per deciliter (g/dL) after the administering.

The present disclosure provides for, and includes, a method for improving oxygen delivery to a hematologic malignancy patient in need thereof comprising administering at least two units of oxygen and carbon dioxide reduced, leukoreduced red blood cells to the hematologic malignancy patient in need thereof, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, leukoreduced red blood cells are administered to the hematologic malignancy patient in need thereof in a single transfusion event, and wherein the blood oxygen level of the hematologic malignancy patient in need thereof is increased by at least 10% after the administering.

The present disclosure provides for, and includes, a method for preventing or reducing transfusion-related sequelae in a hematologic malignancy patient in need thereof comprising administering at least two units of oxygen and carbon dioxide reduced, leukoreduced red blood cells to the hematologic malignancy patient in need thereof, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, leukoreduced red blood cells are administered to the hematologic malignancy patient in need thereof in a single transfusion event, and wherein the occurrence of transfusion-related sequelae is reduced over a 7 day period after the administering compared to a hematologic malignancy patient in need thereof administered at least two units of conventional red blood cells.

The present disclosure provides for, and includes, a method for improving the quality of life of a hematologic malignancy patient in need thereof comprising administering at least two units of oxygen and carbon dioxide reduced, leukoreduced red blood cells to the hematologic malignancy patient in need thereof, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, leukoreduced red blood cells are administered to the hematologic malignancy patient in need thereof in a single transfusion event, and wherein the quality of life of the hematologic malignancy patient in need thereof is improved after the administering as assessed by the health-related quality of life (HRQOL) data of the hematologic malignancy patient in need thereof.

The present disclosure provides for, and includes, use of donor blood to manufacture oxygen and carbon dioxide reduced, leukoreduced red blood cells having an oxygen saturation of (SO₂) of 20% or less and an initial partial pressure of carbon dioxide (pCO₂) of 30 millimeters of mercury (mmHg) or less prior to and during storage for the therapeutic application of reducing the blood transfusion volume for a hematologic malignancy patient in need thereof.

The present disclosure provides for, and includes, use of donor blood to manufacture oxygen and carbon dioxide reduced, leukoreduced red blood cells having an oxygen saturation of (SO₂) of 20% or less and an initial partial pressure of carbon dioxide (pCO₂) of 30 millimeters of mercury (mmHg) or less prior to and during storage for the therapeutic application of increasing the blood transfusion interval for a hematologic malignancy patient in need thereof.

The present disclosure provides for, and includes, use of donor blood to manufacture oxygen and carbon dioxide reduced, leukoreduced red blood cells having an oxygen saturation of (SO₂) of 20% or less and an initial partial pressure of carbon dioxide (pCO₂) of 30 millimeters of mercury (mmHg) or less prior to and during storage for the therapeutic application of reducing the blood transfusion volume, increasing the blood transfusion interval, or both in a patient having myelodysplastic syndromes (MDS) in need thereof.

### BRIEF DESCRIPTION OF THE DRAWINGS

The present disclosure is provided with reference to the accompanying drawings, wherein:

FIG. 1 displays the overall study design for a single center, pilot clinical investigation of chronically transfused patients with hematologic malignancies, who are transfused with hypoxic red blood cells manufactured with a Hemanext ONE system.

FIGS. 2A and 2B show the study design for the treatment of MDS patients.

### DETAILED DESCRIPTION

Methods of the present disclosure provide for, and include, administering oxygen reduced blood to a hematologic malignancy patient in need thereof. In an aspect, the oxygen reduced blood has an initial oxygen saturation (SO₂) of 25% or less and is maintained at an SO₂ of 25% or less for a storage period. In an aspect, the oxygen reduced blood has an initial oxygen saturation (SO₂) of 20% or less and is maintained at an SO₂ of 20% or less for a storage period. In an aspect, the oxygen reduced blood has an initial SO₂ of 15% or less and is maintained at an SO₂ of 15% or less for a storage period. In an aspect, the oxygen reduced blood has an initial SO₂ of 10% or less and is maintained at an SO₂ of 10% or less for a storage period. In an aspect, the oxygen reduced blood has an initial SO₂ of 5% or less and is maintained at an SO₂ of 5% or less for a storage period. In an aspect, the oxygen reduced blood is carbon dioxide reduced. In an aspect, the oxygen reduced blood has an initial partial pressure of carbon dioxide (pCO₂) of 30 millimeters of mercury (mmHg) or less and is maintained at pCO₂ of 30 mmHg or less for a storage period. In an aspect, In an aspect, the oxygen reduced blood has an initial pCO₂ of 20 mmHg or less and is maintained at pCO₂ of 20 mmHg or less for a storage period. In an aspect, In an aspect, the oxygen reduced blood has an initial pCO₂ of 15 mmHg or less and is maintained at pCO₂ of 15 mmHg or less for a storage period. In an aspect, In an aspect, the oxygen reduced blood has an initial pCO₂ of 10 mmHg or less and is maintained at pCO₂ of 10 mmHg or less for a storage period. In an aspect, In an aspect, the oxygen reduced blood has an initial pCO₂ of 5 mmHg or less and is maintained at pCO₂ of 5 mmHg or less for a storage period. In an aspect, oxygen reduced blood is administered to a hematologic malignancy patient in need thereof by transfusion.

Methods of the present disclosure provide for, and include, administering oxygen and carbon dioxide reduced blood to a hematologic malignancy patient in need thereof. In an aspect, the oxygen and carbon dioxide reduced blood has an initial oxygen saturation (SO₂) of 20% or less and an initial partial pressure of carbon dioxide (pCO₂) of 30 millimeters of mercury (mmHg) or less and is maintained at an SO₂ of 20% or less and a pCO₂ of 30 mmHg or less for a storage period. In an aspect, the oxygen and carbon dioxide reduced blood has an initial SO₂ of 20% or less and an initial pCO₂ of 20 mmHg or less and is maintained at an SO₂ of 20% or less and a pCO₂ of 20 mmHg or less for a storage period. In an aspect, the oxygen and carbon dioxide reduced blood has an initial SO₂ of 15% or less and an initial pCO₂ of 20 mmHg or less and is maintained at an SO₂ of 15% or less and a pCO₂ of 20 mmHg or less for a storage period. In an aspect, the oxygen and carbon dioxide reduced blood has an initial SO₂ of 15% or less and an initial pCO₂ of 15 mmHg or less and is maintained at an SO₂ of 15% or less and a pCO₂ of 15 mmHg or less for a storage period. In an aspect, the oxygen and carbon dioxide reduced blood has an initial SO₂ of 10% or less and an initial pCO₂ of 15 mmHg or less and is maintained at an SO₂ of 15% or less and a pCO₂ of 20 mmHg or less for a storage period. In an aspect, the oxygen and carbon dioxide reduced blood has an initial SO₂ of 10% or less and an initial pCO₂ of 10 mmHg or less and is maintained at an SO₂ of 15% or less and a pCO₂ of 10 mmHg or less for a storage period. In an aspect, the oxygen and carbon dioxide reduced blood has an initial SO₂ of 5% or less and an initial pCO₂ of 10 mmHg or less and is maintained at an SO₂ of 5% or less and a pCO₂ of 10 mmHg or less for a storage period. In an aspect, the oxygen and carbon dioxide reduced blood has an initial SO₂ of 5% or less and an initial pCO₂ of 5 mmHg or less and is maintained at an SO₂ of 5% or less and a pCO₂ of 5 mmHg or less for a storage period. In an aspect, oxygen and carbon dioxide reduced blood is administered to a hematologic malignancy patient in need thereof by transfusion.

In an aspect of the present disclosure, oxygen reduced blood is stored for a storage period prior to administration to a hematologic malignancy patient in need thereof. In an aspect, the storage period is less than 2 days, less than 7 days, less than 14 days, less than 21 days, less than 28 days, less than 35 days, less than 42 days, or 42 days. In an aspect, the storage period is 2 days, 7 days, 14 days, 21 days, 28 days, 35 days, or 42 days. In an aspect, the storage period is less than 2 days. In an aspect, the storage period is less than 7 days. In an aspect, the storage period is less than 14 days. In an aspect, the storage period is less than 21 days. In an aspect, the storage period is less than 28 days. In an aspect, the storage period is less than 35 days. In an aspect, the storage period is less than 42 days. In an aspect, the storage period is 42 days.

Suitable blood for use in methods according to the present disclosure comprise oxygen reduced blood having an anticoagulant. In an aspect, oxygen reduced blood further comprises an additive solution. Suitable additive solutions according to the present disclosure include AS-1, AS-3 (Nutricel®), AS-5, CPD, SAGM, PAGG-SM, PAGG-GM, MAP, AS-7, ESOL-5, EAS61, OFAS1, OFAS3, and combinations thereof. In an aspect, the additive solution is added at the time of component separation. In an aspect, the additive solution is AS-1. In an aspect, the additive solution is AS-3. In an aspect, the additive solution is SAGM. In an aspect, the additive solution is citrate-phosphate-dextrose (CPD). In an aspect, the additive solution is phosphate-adenosine-guanosine-glucose-saline-mannitol (PAGG-SM). In an aspect, the additive solution is a mixture of citrate-phosphate-dextrose (CPD) and phosphate-adenosine-guanosine-glucose-saline-mannitol (PAGG-SM).

In an aspect of the present disclosure, a hematologic malignancy patient is in need of a blood transfusion. In an aspect, a hematologic malignancy patient is in need of multiple blood transfusions. In an aspect, a hematologic malignancy patient is in need of periodic blood transfusions. In an aspect, a hematologic malignancy patient is blood transfusion-dependent. As used herein, a blood transfusion-dependent patient is a patient who requires an average of more than two units of blood over a period of at least three weeks. In an aspect, a hematologic malignancy patient has anemia. In an aspect, a hematologic malignancy patient has transfusion-dependent anemia. In an aspect, a hematologic malignancy patient is in need of transfusion with oxygen reduced blood. In an aspect, a hematologic malignancy patient has myelodysplastic syndromes (MDS), MDS with ring sideroblasts (MDS-RS), MDS with deletion of part of the long arm of human chromosome 5q (del5q), acute myeloid leukemia (AML), myelofibrosis, myelomatosis, or leukemia. In an aspect, the myelomatosis is multiple myeloma. In an aspect, the leukemia is acute lymphoblastic leukemia (ALL), chronic lymphocytic leukemia (CLL), acute myelogenous leukemia (AML), or chronic myelogenous leukemia (CML).

In an aspect of the present disclosure, a hematologic malignancy patient in need of a blood transfusion has myelodysplastic syndromes (MDS). As used herein, MDS refers to a group of conditions that can occur when the blood-forming cells in the bone marrow become abnormal and do not properly mature. Signs and symptoms of MDS include anemia (low red blood cell count or reduced hemoglobin), neutropenia (low neutrophil count), and thrombocytopenia (low platelet count). One system for assessing the prognosis of MDS patients is the Revised International Prognostic Scoring System (IPSS-R). A patient's IPSS-R is a weighted score based on hemoglobin count, absolute neutrophil count, platelet count, percentage of bone marrow blasts, and cytogenic category, and IPSS-R scores are separated into five risk categories (very low, low, intermediate, high, or very high). See Greenberg et al., “Revised international prognostic scoring system for myelodysplastic syndromes,” Blood 120(12):2454-2465 (2012). In an aspect, an MDS patient has very low, low, or intermediate risk MDS as determined by the IPSS-R. In an aspect, an MDS patient has very low risk MDS as determined by the IPSS-R. In an aspect, an MDS patient has low risk MDS as determined by the IPSS-R. In an aspect, an MDS patient has intermediate risk MDS as determined by the IPSS-R. In an aspect, a bone marrow aspirate of an MDS patient completed up to six months prior to the administration of oxygen reduced blood did not indicate progression to higher risk MDS. In an aspect, an MDS patient requires at least two units of conventional red blood cells every four weeks prior to the administering.

In an aspect of the present disclosure, an MDS patient has an elevated serum ferritin level prior to being administered oxygen reduced blood. In an aspect, the elevated serum ferritin level prior to being administered oxygen reduced blood is greater than 200 nanograms per milliliter (ng/mL) for a female patient having MDS. In an aspect, the elevated serum ferritin level prior to being administered oxygen reduced blood is greater than 300 ng/mL for a male patient having MDS. In an aspect, the elevated serum ferritin level prior to being administered oxygen reduced blood is greater than 200 nanograms per milliliter (ng/mL) for a female patient having MDS, and greater than 300 ng/mL for a male patient having MDS.

In an aspect, an elevated serum ferritin level in an MDS patient is reduced after being administered oxygen reduced blood. In an aspect, the elevated serum ferritin level in a female MDS patient is reduced to 200 nanograms per milliliter (ng/mL) or less after being administered oxygen reduced blood. In an aspect, the elevated serum ferritin level in a male MDS patient is reduced to 300 ng/mL or less after being administered oxygen reduced blood. In an aspect, the elevated serum ferritin level in a female MDS patient is reduced to 200 ng/mL or less after being administered oxygen reduced blood, and the elevated serum ferritin level in a male MDS patient is reduced to 300 ng/mL or less after being administered oxygen reduced blood. In an aspect, the elevated serum ferritin level in an MDS patient is reduced by at least 5%, at least 10%, at least 15%, at least 20%, at least 25%, or at least 30% after being administered oxygen reduced blood. In an aspect, the elevated serum ferritin level in an MDS patient is reduced by at least 1-fold, at least 2-fold, at least 3-fold, at least 4-fold, or at least 5-fold after being administered oxygen reduced blood. In an aspect, the elevated serum ferritin level in an MDS patient is reduced by at least 5% after being administered oxygen reduced blood compared to the elevated serum ferritin level in an MDS patient after being administered non-oxygen reduced blood. In an aspect, the elevated serum ferritin level in an MDS patient is reduced by at least 10% after being administered oxygen reduced blood compared to the elevated serum ferritin level in an MDS patient after being administered non-oxygen reduced blood. In an aspect, the elevated serum ferritin level in an MDS patient is reduced by at least 15% after being administered oxygen reduced blood compared to the elevated serum ferritin level in an MDS patient after being administered non-oxygen reduced blood. In an aspect, the elevated serum ferritin level in an MDS patient is reduced by at least 20% after being administered oxygen reduced blood compared to the elevated serum ferritin level in an MDS patient after being administered non-oxygen reduced blood. In an aspect, the elevated serum ferritin level in an MDS patient is reduced by at least 25% after being administered oxygen reduced blood compared to the elevated serum ferritin level in an MDS patient after being administered non-oxygen reduced blood. In an aspect, the elevated serum ferritin level in an MDS patient is reduced by at least 30% after being administered oxygen reduced blood compared to the elevated serum ferritin level in an MDS patient after being administered non-oxygen reduced blood. In an aspect, the elevated serum ferritin level in an MDS patient is reduced by at least 1-fold after being administered oxygen reduced blood compared to the elevated serum ferritin level in an MDS patient after being administered non-oxygen reduced blood. In an aspect, the elevated serum ferritin level in an MDS patient is reduced by at least 2-fold after being administered oxygen reduced blood compared to the elevated serum ferritin level in an MDS patient after being administered non-oxygen reduced blood. In an aspect, the elevated serum ferritin level in an MDS patient is reduced by at least 3-fold after being administered oxygen reduced blood compared to the elevated serum ferritin level in an MDS patient after being administered non-oxygen reduced blood. In an aspect, the elevated serum ferritin level in an MDS patient is reduced by at least 4-fold after being administered oxygen reduced blood compared to the elevated serum ferritin level in an MDS patient after being administered non-oxygen reduced blood. In an aspect, the elevated serum ferritin level in an MDS patient is reduced by at least 5-fold after being administered oxygen reduced blood compared to the elevated serum ferritin level in an MDS patient after being administered non-oxygen reduced blood. In an aspect, oxygen reduced blood is oxygen and carbon dioxide reduced blood.

In an aspect of the present disclosure, a hematologic malignancy patient in need of a blood transfusion has MDS-RS. As used herein, RS refers to erythroid precursors with abnormal perinuclear mitochondrial iron accumulation. As used therein, MDS-RS refers to a myeloid neoplasm defined by the presence of RS. The World Health Organization (WHO) criteria for the diagnosis of MDS-RS allows for the diagnosis of MDS-RS either with at least 15% bone marrow RS, or with at least 5% bone marrow RS in the presence of SF3B1 mutations.

In an aspect of the present disclosure, a hematologic malignancy patient in need of a blood transfusion has MDS-del5q. As used therein, MDS-del5q refers to a type of MDS where part of chromosome 5 is missing. In an aspect, MDS-del5q remains stable for many years causing few symptoms. In an aspect, MDS-del5q may progress rapidly into a different subtype of MDS or transform into acute leukemia. MDS-del5q comprises refractory anemia, refractory neutropenia, and refractory thrombocytopenia.

In an aspect of the present disclosure, a hematologic malignancy patient in need of a blood transfusion has Acute Myeloid Leukemia (AML). Not to be limited by theory, AML refers to a cancer of the myeloid line of blood cells, characterized by the rapid growth of abnormal cells that build up in the bone marrow and blood and interfere with normal blood cell production. Signs and symptoms of AML include fatigue, shortness of breath, easy bruising and bleeding, and increased risk of infection.

In an aspect of the present disclosure, a hematologic malignancy patient in need of a blood transfusion has myelofibrosis. Not to be limited by theory, myelofibrosis refers to a type of myeloproliferative neoplasm, a group of cancers in which there is growth of abnormal cells in the bone marrow. Signs and symptoms of myelofibrosis comprise fatigue, easy bruising and bleeding, anemia, increased infection and an enlarged spleen.

In an aspect of the present disclosure, the functional status of a hematologic malignancy patient can be assessed by an Eastern Cooperative Oncology Group (ECOG) performance status scale, which is scored as 0, 1, 2, 3, or 4. See e.g., Oken et al., “Toxicity and response criteria of the Eastern Cooperative Oncology Group,” Am J Clin Oncol. 5(6):649-655 (1982). An ECOG performance status of 0 indicates that the patient is fully active with no performance restrictions. An ECOG performance status of 1 indicates that strenuous physical activity restricted, but the patient is fully ambulatory and able to carry out light work. An ECOG performance status of 2 indicates that the patient is capable of all self-care but is unable to carry out any work activities, and is up and about for more than half of waking hours. An ECOG performance status of 3 indicates that the patient is capable of only limited self-care, and is confined to a bed or chair for more than half of waking hours Finally, an ECOG performance status of 4 indicates that the patient is completely disabled, cannot carry out any self-care, and is confined to bed or chair at all times. In an aspect, a hematologic malignancy patient has an ECOG performance status of 3 or less prior to being administered oxygen reduced blood. In an aspect, a hematologic malignancy patient has an ECOG performance status of 0, 1, 2, or 3 prior to being administered oxygen reduced blood. In an aspect, an MDS patient has an ECOG performance status of 3 or less prior to being administered oxygen reduced blood. In an aspect, an MDS patient has an ECOG performance status of 0, 1, 2, or 3 prior to being administered oxygen reduced blood.

In an aspect, one, two, or three units of oxygen reduced blood are administered to the hematologic malignancy patient. In an aspect, at least one, at least two, or at least three units of oxygen reduced blood are administered to the hematologic malignancy patient. In an aspect, between one to two, between two to three, or between one to three units of oxygen reduced blood are administered to the hematologic malignancy patient. In an aspect, more than three units of oxygen reduced blood are administered to the hematologic malignancy patient. In an aspect, the oxygen reduced blood is administered to the hematologic malignancy patient as a single transfusion event. In an aspect, the oxygen reduced blood is administered to the hematologic malignancy patient as a double transfusion event. In an aspect, the oxygen reduced blood is administered to the hematologic malignancy patient as a multi-transfusion event.

In an aspect of the present disclosure, a hematologic malignancy patient in need of a blood transfusion is administered oxygen reduced blood periodically over a treatment time period. In an aspect, a treatment time period is for at least 7 days, for at least 14 days, for at least 21 days, or for at least 28 days. In an aspect, the treatment time period is for at least 1 month, for at least 1.5 months, for at least 2 months, for at least 3 months, for at least 4 months, for at least 5 month, for at least 6 months, for at least 7 months, for at least 8 months, for at least 9 months, for at least 10 months, or for at least 11 months. In an aspect, the treatment time period is for at least 1 year, for at least 1.5 years, for at least 2 years, for at least 3 years, for at least 4 years, for at least 5 years, for at least 7.5 years, or for at least 10 years.

In an aspect of the present disclosure, a hematologic malignancy patient in need of a blood transfusion is administered oxygen reduced blood over a transfusion time period. In an aspect, the transfusion time period for a single administration of oxygen reduced blood is at least 30 minutes, at least 1 hour, at least 90 minutes, at least 2 hours, at 3 three hours, at least 4 hours.

In an aspect of the present disclosure, a hematologic malignancy patient in need of a blood transfusion is administered oxygen reduced blood every day, every 2 days, every 3 days, every 4 days, every 5 days, every 6 days, every 7 days, every 8 days, every 9 days, every 10 days, every 12 days, every 14 days, every 16 days, every 18 days, every 20 days, every 22 days, every 24 days, every 26 days, every 28 days, or every 30 days. In an aspect, a hematologic malignancy patient in need of a blood transfusion is administered oxygen reduced blood once a week, twice a week, every other week, every 3rd week, every 4th week, every 5th week, every 6^{th} week, every 7^{th} week, every 8^{th} week, every 9^{th} week, and every 10^{th} week. In an aspect, hematologic malignancy patient in need of a blood transfusion is administered oxygen reduced blood on an irregular basis, or on an as-needed basis.

In an aspect of the present disclosure, a hematologic malignancy patient has a hemoglobin level of less than 9 grams per deciliter (g/dL) prior to being administered oxygen reduced blood. In an aspect, a hematologic malignancy patient has a hemoglobin level of less than 8.5 g/dL prior to being administered oxygen reduced blood. In an aspect, a hematologic malignancy patient has a hemoglobin level of less than 8 g/dL prior to being administered oxygen reduced blood. In an aspect, a hematologic malignancy patient has a hemoglobin level of less than 7.5 g/dL prior to being administered oxygen reduced blood. In an aspect, a hematologic malignancy patient has a hemoglobin level of between 8 g/dL to 9 g/dL prior to being administered oxygen reduced blood. In an aspect, a hematologic malignancy patient has a hemoglobin level of about 7.5 g/dL, about 8 g/dL, about 8.5 g/dL, or about 9 g/dL prior to being administered oxygen reduced blood. In an aspect, oxygen reduced blood is oxygen and carbon dioxide reduced blood.

In an aspect of the present disclosure, a hematologic malignancy patient has a hemoglobin level of 9 grams per deciliter (g/dL) or more after being administered oxygen reduced blood. In an aspect, a hematologic malignancy patient has a hemoglobin level of 9.5 g/dL or more after being administered oxygen reduced blood. In an aspect, a hematologic malignancy patient has a hemoglobin level of 10 g/dL or more after being administered o oxygen reduced blood. In an aspect, a hematologic malignancy patient has a hemoglobin level of 11 g/dL or more, 12 g/dL or more, 13 g/dL or more, 14 g/dL or more, 15 g/dL or more, or 16 g/dL or more after being administered oxygen reduced blood. In an aspect, a hematologic malignancy patient has a hemoglobin level of between 9 g/dL to 10 g/dL after being administered oxygen reduced blood. In an aspect, a hematologic malignancy patient has a hemoglobin level of between 9 g/dL to 12 g/dL after being administered oxygen reduced blood. In an aspect, a hematologic malignancy patient has a hemoglobin level of about 9 g/dL, about 9.5 g/dL, about 10 g/dL, about 11 g/dL, about 12 g/dL, about 13 g/dL, about 14 g/dL, or about 15 g/dL after being administered oxygen reduced blood. In an aspect, a hematologic malignancy patient has a normal hemoglobin level after being administered oxygen reduced blood. In an aspect, oxygen reduced blood is oxygen and carbon dioxide reduced blood.

In an aspect of the present disclosure, the hemoglobin level of a hematologic malignancy patient is increased by at least 0.5 grams per deciliter (g/dL) after being administered oxygen reduced blood. In an aspect, the hemoglobin level of a hematologic malignancy patient is increased by at least 1 g/dL after being administered oxygen reduced blood. In an aspect, the hemoglobin level of a hematologic malignancy patient is increased by at least 1.5 g/dL after being administered oxygen reduced blood. In an aspect, the hemoglobin level of a hematologic malignancy patient is increased by at least 2 g/dL after being administered oxygen reduced blood. In an aspect, the hemoglobin level of a hematologic malignancy patient is increased by at least 3 g/dL, at least 4 g/dL, at least 5 g/dL, or at least 6 g/dL after being administered oxygen reduced blood. In an aspect, the hemoglobin level of a hematologic malignancy patient is increased by between 1 g/dL to 2 g/dL after being administered oxygen reduced blood. In an aspect, the hemoglobin level of a hematologic malignancy patient is increased by between 1 g/dL to 3 g/dL after being administered oxygen reduced blood. In an aspect, the hemoglobin level of a hematologic malignancy patient is increased by at about 1 g/dL after being administered oxygen reduced blood. In an aspect, the hemoglobin level of a hematologic malignancy patient is increased by at about 1.5 g/dL after being administered oxygen reduced blood. In an aspect, the hemoglobin level of a hematologic malignancy patient is increased by at about 2 g/dL after being administered oxygen reduced blood. In an aspect, oxygen reduced blood is oxygen and carbon dioxide reduced blood.

In an aspect of the present disclosure, the hemoglobin level of a hematologic malignancy patient is increased by at least 0.5 grams per deciliter (g/dL) after being administered oxygen reduced blood compared to a hematologic malignancy patient having been administered the same volume of non-oxygen reduced blood. In an aspect, the hemoglobin level of a hematologic malignancy patient is increased by at least 1 g/dL after being administered oxygen reduced blood compared to a hematologic malignancy patient having been administered the same volume of non-oxygen reduced blood. In an aspect, the hemoglobin level of a hematologic malignancy patient is increased by at least 1.5 g/dL after being administered oxygen reduced blood compared to a hematologic malignancy patient having been administered the same volume of non-oxygen reduced blood. In an aspect, the hemoglobin level of a hematologic malignancy patient is increased by at least 2 g/dL after being administered oxygen reduced blood compared to a hematologic malignancy patient having been administered the same volume of non-oxygen reduced blood. In an aspect, the hemoglobin level of a hematologic malignancy patient is increased by at least 3 g/dL, at least 4 g/dL, at least 5 g/dL, or at least 6 g/dL after being administered oxygen reduced blood compared to a hematologic malignancy patient having been administered the same volume of non-oxygen reduced blood. In an aspect, the hemoglobin level of a hematologic malignancy patient is increased by between 1 g/dL to 2 g/dL after being administered oxygen reduced blood compared to a hematologic malignancy patient having been administered the same volume of non-oxygen reduced blood. In an aspect, the hemoglobin level of a hematologic malignancy patient is increased by between 1 g/dL to 3 g/dL after being administered oxygen reduced blood compared to a hematologic malignancy patient having been administered the same volume of non-oxygen reduced blood. In an aspect, the hemoglobin level of a hematologic malignancy patient is increased by at about 1 g/dL after being administered oxygen reduced blood compared to a hematologic malignancy patient having been administered the same volume of non-oxygen reduced blood. In an aspect, the hemoglobin level of a hematologic malignancy patient is increased by at about 1.5 g/dL after being administered oxygen reduced blood compared to a hematologic malignancy patient having been administered the same volume of non-oxygen reduced blood. In an aspect, the hemoglobin level of a hematologic malignancy patient is increased by at about 2 g/dL after being administered oxygen reduced blood compared to a hematologic malignancy patient having been administered the same volume of non-oxygen reduced blood. In an aspect, oxygen reduced blood is oxygen and carbon dioxide reduced blood.

In an aspect, the hemoglobin level of a hematologic malignancy patient is increased by at least 5%, at least 10%, at least 15%, at least 20%, at least 25%, or at least 30% after being administered oxygen reduced blood. In an aspect, the hemoglobin level of a hematologic malignancy patient is increased by at least 1-fold, at least 2-fold, at least 3-fold, at least 4-fold, or at least 5-fold after being administered oxygen reduced blood. In an aspect, the hemoglobin level of a hematologic malignancy patient is increased by at least 5% after being administered oxygen reduced blood compared to a hematologic malignancy patient having been administered the same volume of non-oxygen reduced blood. In an aspect, the hemoglobin level of a hematologic malignancy patient is increased by at least 10% after being administered oxygen reduced blood compared to a hematologic malignancy patient having been administered the same volume of non-oxygen reduced blood. In an aspect, at least 15% after being administered oxygen reduced blood compared to a hematologic malignancy patient having been administered the same volume of non-oxygen reduced blood. In an aspect, the hemoglobin level of a hematologic malignancy patient is increased by at least 20% after being administered oxygen reduced blood compared to a hematologic malignancy patient having been administered the same volume of non-oxygen reduced blood. In an aspect, the hemoglobin level of a hematologic malignancy patient is increased by at least 25% after being administered oxygen reduced blood compared to a hematologic malignancy patient having been administered the same volume of non-oxygen reduced blood. In an aspect, the hemoglobin level of a hematologic malignancy patient is increased by at least 30% after being administered oxygen reduced blood compared to a hematologic malignancy patient having been administered the same volume of non-oxygen reduced blood. In an aspect, the hemoglobin level of a hematologic malignancy patient is increased by at least 1-fold after being administered oxygen reduced blood compared to a hematologic malignancy patient having been administered the same volume of non-oxygen reduced blood. In an aspect, the hemoglobin level of a hematologic malignancy patient is increased by at least 2-fold after being administered oxygen reduced blood compared to a hematologic malignancy patient having been administered the same volume of non-oxygen reduced blood. In an aspect, the hemoglobin level of a hematologic malignancy patient is increased by at least 3-fold after being administered oxygen reduced blood compared to a hematologic malignancy patient having been administered the same volume of non-oxygen reduced blood. In an aspect, the hemoglobin level of a hematologic malignancy patient is increased by at least 4-fold after being administered oxygen reduced blood compared to a hematologic malignancy patient having been administered the same volume of non-oxygen reduced blood. In an aspect, the hemoglobin level of a hematologic malignancy patient is increased by at least 5-fold after being administered oxygen reduced blood compared to a hematologic malignancy patient having been administered the same volume of non-oxygen reduced blood. In an aspect, oxygen reduced blood is oxygen and carbon dioxide reduced blood.

Methods of the present disclosure provide for, and include, increasing the hemoglobin increment of a hematologic malignancy patient in need thereof by administering at least two units of oxygen reduced blood, as provided herein, to the hematologic malignancy patient. As used herein, a hemoglobin increment is the change in hemoglobin per unit of transfused blood. In an aspect, the hemoglobin increment of the hematologic malignancy patient is increased by at least 0.5 grams per deciliter (g/dL) after being administered oxygen reduced blood. In an aspect, the hemoglobin increment of the hematologic malignancy patient is increased by at least 1 g/dL after being administered oxygen reduced blood. In an aspect, the hemoglobin increment of the hematologic malignancy patient is increased by at least 1.5 g/dL after being administered oxygen reduced blood. In an aspect, the hemoglobin increment of the hematologic malignancy patient is increased by at least 2 g/dL after being administered oxygen reduced blood. In an aspect, the hemoglobin increment of the hematologic malignancy patient is increased by at least 2.5 g/dL after being administered oxygen reduced blood. In an aspect, the hemoglobin increment of the hematologic malignancy patient is increased by between 0.5 to 1 g/dL after being administered oxygen reduced blood. In an aspect, the hemoglobin increment of the hematologic malignancy patient is increased by between 1 to 2 g/dL after being administered oxygen reduced blood. In an aspect, the hemoglobin increment of the hematologic malignancy patient is increased by one-fold after being administered oxygen reduced blood. In an aspect, the hemoglobin increment of the hematologic malignancy patient is increased by two-fold after being administered oxygen reduced blood. In an aspect, the hemoglobin increment of the hematologic malignancy patient is increased by three-fold after being administered oxygen reduced blood. In an aspect, the hemoglobin increment of the hematologic malignancy patient is increased by four-fold after being administered oxygen reduced blood. In an aspect, the hemoglobin increment of the hematologic malignancy patient is increased by five-fold after being administered oxygen reduced blood. In an aspect, iron overload is reduced in the hematologic malignancy patient after being administered oxygen reduced blood.

In an aspect of the present disclosure, the neutrophil concentration of a hematologic malignancy patient is less than 1.8×10⁹/L prior to being administered oxygen reduced blood. In an aspect, the neutrophil concentration of a hematologic malignancy patient is less than 1.6×10⁹/L prior to being administered oxygen reduced blood. In an aspect, the neutrophil concentration of a hematologic malignancy patient is less than 1.4×10⁹/L prior to being administered oxygen reduced blood. In an aspect, the neutrophil concentration of a hematologic malignancy patient is less than 1.2×10⁹/L prior to being administered oxygen reduced blood. In an aspect, the neutrophil concentration of a hematologic malignancy patient is less than 1.0×10⁹/L prior to being administered oxygen reduced blood. In an aspect, the neutrophil concentration of a hematologic malignancy patient is between 1.2×10⁹/L to 1.6×10⁹/L prior to being administered oxygen reduced blood. In an aspect, the neutrophil concentration of a hematologic malignancy patient is between 1.2×10⁹/L to 1.6×10⁹/L prior to being administered oxygen reduced blood. In an aspect, the neutrophil concentration of a hematologic malignancy patient is between 1.0×10⁹/L to 1.6×10⁹/L prior to being administered oxygen reduced blood. In an aspect, oxygen reduced blood is oxygen and carbon dioxide reduced blood.

In an aspect of the present disclosure, the neutrophil concentration of a hematologic malignancy patient is 1.8×10⁹/L or more after being administered oxygen reduced blood. In an aspect, the neutrophil concentration of the hematologic malignancy patient is 2.0×10⁹/L or more after being administered oxygen reduced blood. In an aspect, the neutrophil concentration of the hematologic malignancy patient is 2.5×10⁹/L or more after being administered oxygen reduced blood. In an aspect, the neutrophil concentration of the hematologic malignancy patient is 3.0×10⁹/L or more after being administered oxygen reduced blood. In an aspect, the neutrophil concentration of the hematologic malignancy patient is 4.0×10⁹/L or more after being administered oxygen reduced blood. In an aspect, the neutrophil concentration of the hematologic malignancy patient is 5.0×10⁹/L or more after being administered oxygen reduced blood. In an aspect, the neutrophil concentration of the hematologic malignancy patient is 6.0×10⁹/L or more after being administered oxygen reduced blood. In an aspect, the neutrophil concentration of a hematologic malignancy patient is between 3.0×10⁹/L to 5.0×10⁹/L after being administered oxygen reduced blood. In an aspect, the neutrophil concentration of a hematologic malignancy patient is between 3.0×10⁹/L to 7.0×10⁹/L after being administered oxygen reduced blood. In an aspect, the neutrophil concentration of a hematologic malignancy patient is between 4.0×10⁹/L to 5.0×10⁹/L after being administered oxygen reduced blood. In an aspect, the neutrophil concentration of a hematologic malignancy patient is between 4.0×10⁹/L to 6.0×10⁹/L after being administered oxygen reduced blood. In an aspect, the neutrophil concentration of a hematologic malignancy patient is between 4.0×10⁹/L to 7.0×10⁹/L after being administered oxygen reduced blood. In an aspect, the neutrophil concentration of a hematologic malignancy patient is between 5.0×10⁹/L to 7.0×10⁹/L after being administered oxygen reduced blood. In an aspect, a hematologic malignancy patient has a neutrophil concentration that is within normal range after being administered oxygen reduced blood. In an aspect, oxygen reduced blood is oxygen and carbon dioxide reduced blood.

In an aspect of the present disclosure, the neutrophil concentration of a hematologic malignancy patient is increased by at least 0.2×10⁹/L after being administered oxygen reduced blood. In an aspect, the neutrophil concentration of a hematologic malignancy patient is increased by at least 0.4×10⁹/L after being administered oxygen reduced blood. In an aspect, the neutrophil concentration of a hematologic malignancy patient is increased by at least 0.8×10⁹/L after being administered oxygen reduced blood. In an aspect, the neutrophil concentration of a hematologic malignancy patient is increased by at least 1.0×10⁹/L after being administered oxygen reduced blood. In an aspect, the neutrophil concentration of a hematologic malignancy patient is increased by at least 1.5×10⁹/L after being administered oxygen reduced blood. In an aspect, the neutrophil concentration of a hematologic malignancy patient is increased by at least 2.0×10⁹/L after being administered oxygen reduced blood. In an aspect, the neutrophil concentration of a hematologic malignancy patient is increased by at least 2.5×10⁹/L after being administered oxygen reduced blood. In an aspect, the neutrophil concentration of a hematologic malignancy patient is increased by at least 3.0×10⁹/L after being administered oxygen reduced blood. In an aspect, the neutrophil concentration of a hematologic malignancy patient is increased by at least 4.0×10⁹/L after being administered oxygen reduced blood. In an aspect, the neutrophil concentration of a hematologic malignancy patient is increased by between 0.2×10⁹/L to 1.0×10⁹/L after being administered oxygen reduced blood. In an aspect, the neutrophil concentration of a hematologic malignancy patient is increased by between 0.2×10⁹/L to 5.0×10⁹/L after being administered oxygen reduced blood. In an aspect, the neutrophil concentration of a hematologic malignancy patient is increased by between 0.5×10⁹/L to 1.5×10⁹/L after being administered oxygen reduced blood. In an aspect, the neutrophil concentration of a hematologic malignancy patient is increased by between 0.5×10⁹/L to 5.0×10⁹/L after being administered oxygen reduced blood. In an aspect, the neutrophil concentration of a hematologic malignancy patient is increased by between 1.0×10⁹/L to 5.0×10⁹/L after being administered oxygen reduced blood. In an aspect, oxygen reduced blood is oxygen and carbon dioxide reduced blood.

In an aspect, the neutrophil concentration of a hematologic malignancy patient is increased by at least 5%, at least 10%, at least 15%, at least 20%, at least 25%, or at least 30% after being administered oxygen reduced blood. In an aspect, the neutrophil concentration of a hematologic malignancy patient is increased by at least 1-fold, at least 2-fold, at least 3-fold, at least 4-fold, or at least 5-fold after being administered oxygen reduced blood. In an aspect, the neutrophil cell concentration of a hematologic malignancy patient is increased by at least 5% after being administered oxygen reduced blood compared to a hematologic malignancy patient having been administered the same volume of non-oxygen reduced blood. In an aspect, the neutrophil concentration of a hematologic malignancy patient is increased by at least 10% after being administered oxygen reduced blood compared to a hematologic malignancy patient having been administered the same volume of non-oxygen reduced blood. In an aspect, the neutrophil concentration of a hematologic malignancy patient is increased by at least 15% after being administered oxygen reduced blood compared to a hematologic malignancy patient having been administered the same volume of non-oxygen reduced blood. In an aspect, the neutrophil concentration of a hematologic malignancy patient is increased by at least 20% after being administered oxygen reduced blood compared to a hematologic malignancy patient having been administered the same volume of non-oxygen reduced blood. In an aspect, the neutrophil concentration of a hematologic malignancy patient is increased by at least 25% after being administered oxygen reduced blood compared to a hematologic malignancy patient having been administered the same volume of non-oxygen reduced blood. In an aspect, the neutrophil concentration of a hematologic malignancy patient is increased by at least 30% after being administered oxygen reduced blood compared to a hematologic malignancy patient having been administered the same volume of non-oxygen reduced blood. In an aspect, the neutrophil concentration of a hematologic malignancy patient is increased by at least 1-fold after being administered oxygen reduced blood compared to a hematologic malignancy patient having been administered the same volume of non-oxygen reduced blood. In an aspect, the neutrophil concentration of a hematologic malignancy patient is increased by at least 2-fold after being administered oxygen reduced blood compared to a hematologic malignancy patient having been administered the same volume of non-oxygen reduced blood. In an aspect, the neutrophil concentration of a hematologic malignancy patient is increased by at least 3-fold after being administered oxygen reduced blood compared to a hematologic malignancy patient having been administered the same volume of non-oxygen reduced blood. In an aspect, the neutrophil concentration of a hematologic malignancy patient is increased by at least 4-fold after being administered oxygen reduced blood compared to a hematologic malignancy patient having been administered the same volume of non-oxygen reduced blood. In an aspect, the neutrophil concentration of a hematologic malignancy patient is increased by at least 5-fold after being administered oxygen reduced blood compared to a hematologic malignancy patient having been administered the same volume of non-oxygen reduced blood. In an aspect, oxygen reduced blood is oxygen and carbon dioxide reduced blood.

In an aspect of the present disclosure, the total white blood cell concentration of a hematologic malignancy patient is less than 4.0×10⁹/L prior to being administered oxygen reduced blood. In an aspect, the total white blood cell concentration of a hematologic malignancy patient is less than 3.5×10⁹/L prior to being administered oxygen reduced blood. In an aspect, the total white blood cell concentration of a hematologic malignancy patient is less than 3.0×10⁹/L prior to being administered oxygen reduced blood. In an aspect, the total white blood cell concentration of a hematologic malignancy patient is between 3.0×10⁹/L to 4.0×10⁹/L prior to being administered oxygen reduced blood. In an aspect, the total white blood cell concentration of a hematologic malignancy patient is between 2.0×10⁹/L to 4.0×10⁹/L prior to being administered oxygen reduced blood. In an aspect, the total white blood cell concentration of a hematologic malignancy patient is about 3.0×10⁹/L prior to being administered oxygen reduced blood. In an aspect, oxygen reduced blood is oxygen and carbon dioxide reduced blood.

In an aspect of the present disclosure, the total white blood cell concentration of a hematologic malignancy patient is 4.0×10⁹/L or more after being administered oxygen reduced blood. In an aspect, the total white blood cell concentration of the hematologic malignancy patient is 4.5×10⁹/L or more after being administered oxygen reduced blood. In an aspect, the total white blood cell concentration of the hematologic malignancy patient is 5.0×10⁹/L or more after being administered oxygen reduced blood. In an aspect, the total white blood cell concentration of the hematologic malignancy patient is 6.0×10⁹/L or more after being administered oxygen reduced blood. In an aspect, the total white blood cell concentration of the hematologic malignancy patient is 7.0×10⁹/L or more after being administered oxygen reduced blood. In an aspect, the total white blood cell concentration of the hematologic malignancy patient is 8.0×10⁹/L or more after being administered oxygen reduced blood. In an aspect, the total white blood cell concentration of the hematologic malignancy patient is 9.0×10⁹/L or more after being administered oxygen reduced blood. In an aspect, the total white blood cell concentration of a hematologic malignancy patient is between 4.0×10⁹/L to 5.0×10⁹/L after being administered oxygen reduced blood. In an aspect, the total white blood cell concentration of a hematologic malignancy patient is between 4.0×10⁹/L to 6.0×10⁹/L after being administered oxygen reduced blood. In an aspect, the total white blood cell concentration of a hematologic malignancy patient is between 4.0×10⁹/L to 8.0×10⁹/L after being administered oxygen reduced blood. In an aspect, the total white blood cell concentration of a hematologic malignancy patient is between 5.0×10⁹/L to 7.0×10⁹/L after being administered oxygen reduced blood. In an aspect, the total white blood cell concentration of a hematologic malignancy patient is between 4.0×10⁹/L to 10.0×10⁹/L after being administered oxygen reduced blood. In an aspect, a hematologic malignancy patient has a total white blood cell concentration that is within normal range after being administered oxygen reduced blood. In an aspect, oxygen reduced blood is oxygen and carbon dioxide reduced blood.

In an aspect of the present disclosure, the total white blood cell concentration of a hematologic malignancy patient is increased by at least 0.2×10⁹/L after being administered oxygen reduced blood. In an aspect, the total white blood cell concentration of a hematologic malignancy patient is increased by at least 0.5×10⁹/L after being administered oxygen reduced blood. In an aspect, the total white blood cell concentration of a hematologic malignancy patient is increased by at least 0.7×10⁹/L after being administered oxygen reduced blood. In an aspect, the total white blood cell concentration of a hematologic malignancy patient is increased by at least 1.0×10⁹/L after being administered oxygen reduced blood. In an aspect, the total white blood cell concentration of a hematologic malignancy patient is increased by at least 1.5×10⁹/L after being administered oxygen reduced blood. In an aspect, the total white blood cell concentration of a hematologic malignancy patient is increased by at least 2.0×10⁹/L after being administered oxygen reduced blood. In an aspect, the total white blood cell concentration of a hematologic malignancy patient is increased by between 0.2×10⁹/L to 0.5×10⁹/L after being administered oxygen reduced blood. In an aspect, the total white blood cell concentration of a hematologic malignancy patient is increased by between 0.2×10⁹/L to 1.0×10⁹/L after being administered oxygen reduced blood. In an aspect, the total white blood cell concentration of a hematologic malignancy patient is increased by between 0.2×10⁹/L to 5.0×10⁹/L after being administered oxygen reduced blood. In an aspect, the total white blood cell concentration of a hematologic malignancy patient is increased by between 0.2×10⁹/L to 10.0×10⁹/L after being administered oxygen reduced blood. In an aspect, the total white blood cell concentration of a hematologic malignancy patient is increased by between 0.5×10⁹/L to 1.0×10⁹/L after being administered oxygen reduced blood. In an aspect, the total white blood cell concentration of a hematologic malignancy patient is increased by between 0.5×10⁹/L to 2.0×10⁹/L after being administered oxygen reduced blood. In an aspect, the total white blood cell concentration of a hematologic malignancy patient is increased by between 0.5×10⁹/L to 5.0×10⁹/L after being administered oxygen reduced blood. In an aspect, the total white blood cell concentration of a hematologic malignancy patient is increased by between 0.5×10⁹/L to 10.0×10⁹/L after being administered oxygen reduced blood. In an aspect, the total white blood cell concentration of a hematologic malignancy patient is increased by between 1.0×10⁹/L to 2.0×10⁹/L after being administered oxygen reduced blood. In an aspect, the total white blood cell concentration of a hematologic malignancy patient is increased by between 1.0×10⁹/L to 5.0×10⁹/L after being administered oxygen reduced blood. In an aspect, the total white blood cell concentration of a hematologic malignancy patient is increased by between 1.0×10⁹/L to 10.0×10⁹/L after being administered oxygen reduced blood. In an aspect, the total white blood cell concentration of a hematologic malignancy patient is increased by between 5.0×10⁹/L to 10.0×10⁹/L after being administered oxygen reduced blood. In an aspect, oxygen reduced blood is oxygen and carbon dioxide reduced blood.

In an aspect, the total white blood cell concentration of a hematologic malignancy patient is increased by at least 5%, at least 10%, at least 15%, at least 20%, at least 25%, or at least 30% after being administered oxygen reduced blood. In an aspect, the total white blood cell concentration of a hematologic malignancy patient is increased by at least 1-fold, at least 2-fold, at least 3-fold, at least 4-fold, or at least 5-fold after being administered oxygen reduced blood. In an aspect, the total white blood cell concentration of a hematologic malignancy patient is increased by at least 5% after being administered oxygen reduced blood compared to a hematologic malignancy patient having been administered the same volume of oxygen reduced blood. In an aspect, the total white blood cell concentration of a hematologic malignancy patient is increased by at least 10% after being administered oxygen reduced blood compared to a hematologic malignancy patient having been administered the same volume of oxygen reduced blood. In an aspect, the total white blood cell concentration of a hematologic malignancy patient is increased by at least 15% after being administered oxygen reduced blood compared to a hematologic malignancy patient having been administered the same volume of oxygen reduced blood. In an aspect, the total white blood cell concentration of a hematologic malignancy patient is increased by at least 20% after being administered oxygen reduced blood compared to a hematologic malignancy patient having been administered the same volume of oxygen reduced blood. In an aspect, the total white blood cell concentration of a hematologic malignancy patient is increased by at least 25% after being administered oxygen reduced blood compared to a hematologic malignancy patient having been administered the same volume of oxygen reduced blood. In an aspect, the total white blood cell concentration of a hematologic malignancy patient is increased by at least 30% after being administered oxygen reduced blood compared to a hematologic malignancy patient having been administered the same volume of oxygen reduced blood. In an aspect, the total white blood cell concentration of a hematologic malignancy patient is increased by at least 1-fold after being administered oxygen reduced blood compared to a hematologic malignancy patient having been administered the same volume of oxygen reduced blood. In an aspect, the total white blood cell concentration of a hematologic malignancy patient is increased by at least 2-fold after being administered oxygen reduced blood compared to a hematologic malignancy patient having been administered the same volume of oxygen reduced blood. In an aspect, the total white blood cell concentration of a hematologic malignancy patient is increased by at least 3-fold after being administered oxygen reduced blood compared to a hematologic malignancy patient having been administered the same volume of oxygen reduced blood. In an aspect, the total white blood cell concentration of a hematologic malignancy patient is increased by at least 4-fold after being administered oxygen reduced blood compared to a hematologic malignancy patient having been administered the same volume of oxygen reduced blood. In an aspect, the total white blood cell concentration of a hematologic malignancy patient is increased by at least 5-fold after being administered oxygen reduced blood compared to a hematologic malignancy patient having been administered the same volume of oxygen reduced blood. In an aspect, oxygen reduced blood is oxygen and carbon dioxide reduced blood.

In an aspect of the present disclosure, the thrombocyte concentration of a hematologic malignancy patient is less than 155×10⁹/L prior to being administered oxygen reduced blood. In an aspect, the thrombocyte concentration of a hematologic malignancy patient is less than 145×10⁹/L prior to being administered oxygen reduced blood. In an aspect, the thrombocyte concentration of a hematologic malignancy patient is less than 135×10⁹/L prior to being administered oxygen reduced blood. In an aspect, the thrombocyte concentration of a hematologic malignancy patient is less than 125×10⁹/L prior to being administered oxygen reduced blood. In an aspect, the thrombocyte concentration of a hematologic malignancy patient is less than 115×10⁹/L prior to being administered oxygen reduced blood. In an aspect, the thrombocyte concentration of a hematologic malignancy patient is between 110×10⁹/L to 155×10⁹/L prior to being administered oxygen reduced blood. In an aspect, the thrombocyte concentration of a hematologic malignancy patient is between 100×10⁹/L to 145×10⁹/L prior to being administered oxygen reduced blood. In an aspect, oxygen reduced blood is oxygen and carbon dioxide reduced blood.

In an aspect of the present disclosure, the thrombocyte concentration of a hematologic malignancy patient is 145×10⁹/L or more after being administered oxygen reduced blood. In an aspect, the thrombocyte concentration of the hematologic malignancy patient is 155×10⁹/L or more after being administered oxygen reduced blood. In an aspect, the thrombocyte concentration of the hematologic malignancy patient is 165×10⁹/L or more after being administered oxygen reduced blood. In an aspect, the thrombocyte concentration of the hematologic malignancy patient is 180×10⁹/L or more after being administered oxygen reduced blood. In an aspect, the thrombocyte concentration of the hematologic malignancy patient is 200×10⁹/L or more after being administered oxygen reduced blood. In an aspect, the thrombocyte concentration of the hematologic malignancy patient is 250×10⁹/L or more after being administered oxygen reduced blood. In an aspect, the thrombocyte concentration of the hematologic malignancy patient is 300×10⁹/L or more after being administered oxygen reduced blood. In an aspect, the thrombocyte concentration of a hematologic malignancy patient is between 145×10⁹/L to 200×10⁹/L after being administered oxygen reduced blood. In an aspect, the thrombocyte concentration of a hematologic malignancy patient is between 145×10⁹/L to 300×10⁹/L after being administered oxygen reduced blood. In an aspect, the thrombocyte concentration of a hematologic malignancy patient is between 200×10⁹/L to 300×10⁹/L after being administered oxygen reduced blood. In an aspect, the thrombocyte concentration of a hematologic malignancy patient is between 200×10⁹/L to 350×10⁹/L after being administered oxygen reduced blood. In an aspect, the thrombocyte concentration of a hematologic malignancy patient is between 150×10⁹/L to 350×10⁹/L after being administered oxygen reduced blood. In an aspect, a hematologic malignancy patient has a thrombocyte concentration that is within normal range after being administered oxygen reduced blood. In an aspect, oxygen reduced blood is oxygen and carbon dioxide reduced blood.

In an aspect of the present disclosure, the thrombocyte concentration of a hematologic malignancy patient is increased by at least 10×10⁹/L after being administered oxygen reduced blood. In an aspect, the thrombocyte concentration of a hematologic malignancy patient is increased by at least 20×10⁹/L after being administered oxygen reduced blood. In an aspect, the thrombocyte concentration of a hematologic malignancy patient is increased by at least 30×10⁹/L after being administered oxygen reduced blood. In an aspect, the thrombocyte concentration of a hematologic malignancy patient is increased by at least 40×10⁹/L after being administered oxygen reduced blood. In an aspect, the thrombocyte concentration of a hematologic malignancy patient is increased by at least 50×10⁹/L after being administered oxygen reduced blood. In an aspect, the thrombocyte concentration of a hematologic malignancy patient is increased by at least 75×10⁹/L after being administered oxygen reduced blood. In an aspect, the thrombocyte concentration of a hematologic malignancy patient is increased by at least 100×10⁹/L after being administered oxygen reduced blood. In an aspect, the thrombocyte concentration of a hematologic malignancy patient is increased by at least 150×10⁹/L after being administered oxygen reduced blood. In an aspect, the thrombocyte concentration of a hematologic malignancy patient is increased by between 10×10⁹/L to 50×10⁹/L after being administered oxygen reduced blood. In an aspect, the thrombocyte concentration of a hematologic malignancy patient is increased by between 50×10⁹/L to 100×10⁹/L after being administered oxygen reduced blood. In an aspect, the thrombocyte concentration of a hematologic malignancy patient is increased by between 10×10⁹/L to 100×10⁹/L after being administered oxygen reduced blood. In an aspect, the thrombocyte concentration of a hematologic malignancy patient is increased by between 50×10⁹/L to 200×10⁹/L after being administered oxygen reduced blood. In an aspect, oxygen reduced blood is oxygen and carbon dioxide reduced blood.

In an aspect, the thrombocyte concentration of a hematologic malignancy patient is increased by at least 5%, at least 10%, at least 15%, at least 20%, at least 25%, or at least 30% after being administered oxygen reduced blood. In an aspect, the thrombocyte concentration of a hematologic malignancy patient is increased by at least 1-fold, at least 2-fold, at least 3-fold, at least 4-fold, or at least 5-fold after being administered oxygen reduced blood. In an aspect, the thrombocyte concentration of a hematologic malignancy patient is increased by at least 5% after being administered oxygen reduced blood compared to a hematologic malignancy patient having been administered the same volume of oxygen reduced blood. In an aspect, the thrombocyte concentration of a hematologic malignancy patient is increased by at least 10% after being administered oxygen reduced blood compared to a hematologic malignancy patient having been administered the same volume of oxygen reduced blood. In an aspect, the thrombocyte concentration of a hematologic malignancy patient is increased by at least 15% after being administered oxygen reduced blood compared to a hematologic malignancy patient having been administered the same volume of oxygen reduced blood. In an aspect, the thrombocyte concentration of a hematologic malignancy patient is increased by at least 20% after being administered oxygen reduced blood compared to a hematologic malignancy patient having been administered the same volume of oxygen reduced blood. In an aspect, the thrombocyte concentration of a hematologic malignancy patient is increased by at least 25% after being administered oxygen reduced blood compared to a hematologic malignancy patient having been administered the same volume of oxygen reduced blood. In an aspect, the thrombocyte concentration of a hematologic malignancy patient is increased by at least 30% after being administered oxygen reduced blood compared to a hematologic malignancy patient having been administered the same volume of oxygen reduced blood. In an aspect, the thrombocyte concentration of a hematologic malignancy patient is increased by at least 1-fold after being administered oxygen reduced blood compared to a hematologic malignancy patient having been administered the same volume of oxygen reduced blood. In an aspect, the thrombocyte concentration of a hematologic malignancy patient is increased by at least 2-fold after being administered oxygen reduced blood compared to a hematologic malignancy patient having been administered the same volume of oxygen reduced blood. In an aspect, the thrombocyte concentration of a hematologic malignancy patient is increased by at least 3-fold after being administered oxygen reduced blood compared to a hematologic malignancy patient having been administered the same volume of oxygen reduced blood. In an aspect, the thrombocyte concentration of a hematologic malignancy patient is increased by at least 4-fold after being administered oxygen reduced blood compared to a hematologic malignancy patient having been administered the same volume of oxygen reduced blood. In an aspect, the thrombocyte concentration of a hematologic malignancy patient is increased by at least 5-fold after being administered oxygen reduced blood compared to a hematologic malignancy patient having been administered the same volume of oxygen reduced blood. In an aspect, oxygen reduced blood is oxygen and carbon dioxide reduced blood.

In an aspect of the present disclosure, the reticulocyte concentration of a hematologic malignancy patient is less than 0.020×10¹²/L prior to being administered oxygen reduced blood. In an aspect, the reticulocyte concentration of a hematologic malignancy patient is less than 0.025×10¹²/L prior to being administered oxygen reduced blood. In an aspect, the reticulocyte concentration of a hematologic malignancy patient is less than 0.030×10¹²/L prior to being administered oxygen reduced blood. In an aspect, the reticulocyte concentration of a hematologic malignancy patient is less than 0.035×10¹²/L prior to being administered oxygen reduced blood. In an aspect, the reticulocyte concentration of a hematologic malignancy patient is less than 0.040×10¹²/L prior to being administered oxygen reduced blood. In an aspect, the reticulocyte concentration of a hematologic malignancy patient is less than 0.050×10¹²/L prior to being administered oxygen reduced blood. In an aspect, the reticulocyte concentration of a hematologic malignancy patient is between 0.020×10¹²/L to 0.030×10¹²/L prior to being administered oxygen reduced blood. In an aspect, the reticulocyte concentration of a hematologic malignancy patient is between 0.025×10¹²/L to 0.035×10¹²/L prior to being administered oxygen reduced blood. In an aspect, the reticulocyte concentration of a hematologic malignancy patient is between 0.030×10¹²/L to 0.040×10¹²/L prior to being administered oxygen reduced blood. In an aspect, the reticulocyte concentration of a hematologic malignancy patient is between 0.020×10¹²/L to 0.040×10¹²/L prior to being administered oxygen reduced blood. In an aspect, the reticulocyte concentration of a hematologic malignancy patient is between 0.010×10¹²/L to 0.050×10¹²/L prior to being administered oxygen reduced blood. In an aspect, oxygen reduced blood is oxygen and carbon dioxide reduced blood.

In an aspect of the present disclosure, the reticulocyte concentration of a hematologic malignancy patient is 0.030×10¹²/L or more after being administered oxygen reduced blood. In an aspect, the reticulocyte concentration of the hematologic malignancy patient is 0.035×10¹²/L or more after being administered oxygen reduced blood. In an aspect, the reticulocyte concentration of the hematologic malignancy patient is 0.040×10¹²/L or more after being administered oxygen reduced blood. In an aspect, the reticulocyte concentration of the hematologic malignancy patient is 0.050×10¹²/L or more after being administered oxygen reduced blood. In an aspect, the reticulocyte concentration of the hematologic malignancy patient is 0.060×10¹²/L or more after being administered oxygen reduced blood. In an aspect, the reticulocyte concentration of the hematologic malignancy patient is 0.075×10¹²/L or more after being administered oxygen reduced blood. In an aspect, the reticulocyte concentration of a hematologic malignancy patient is between 0.030×10¹²/L to 0.040×10¹²/L after being administered oxygen reduced blood. In an aspect, the reticulocyte concentration of a hematologic malignancy patient is between 0.030×10¹²/L to 0.050×10¹²/L after being administered oxygen reduced blood. In an aspect, the reticulocyte concentration of a hematologic malignancy patient is between 0.030×10¹²/L to 0.100×10¹²/L after being administered oxygen reduced blood. In an aspect, the reticulocyte concentration of a hematologic malignancy patient is between 0.030×10¹²/L to 0.040×10¹²/L after being administered oxygen reduced blood. In an aspect, the reticulocyte concentration of a hematologic malignancy patient is between 0.040×10¹²/L to 0.050×10¹²/L after being administered oxygen reduced blood. In an aspect, the reticulocyte concentration of a hematologic malignancy patient is between 0.040×10¹²/L to 0.100×10¹²/L after being administered oxygen reduced blood. In an aspect, the reticulocyte concentration of a hematologic malignancy patient is between 0.050×10¹²/L to 0.100×10¹²/L after being administered oxygen reduced blood. In an aspect, a hematologic malignancy patient has a reticulocyte concentration that is within normal range after being administered oxygen reduced blood. In an aspect, oxygen reduced blood is oxygen and carbon dioxide reduced blood.

In an aspect of the present disclosure, the reticulocyte concentration of a hematologic malignancy patient is increased by at least 0.005×10¹²/L after being administered oxygen reduced blood. In an aspect, the reticulocyte concentration of a hematologic malignancy patient is increased by at least 0.010×10¹²/L after being administered oxygen reduced blood. In an aspect, the reticulocyte concentration of a hematologic malignancy patient is increased by at least 0.015×10¹²/L after being administered oxygen reduced blood. In an aspect, the reticulocyte concentration of a hematologic malignancy patient is increased by at least 0.020×10¹²/L after being administered oxygen reduced blood. In an aspect, the reticulocyte concentration of a hematologic malignancy patient is increased by at least 0.030×10¹²/L after being administered oxygen reduced blood. In an aspect, the reticulocyte concentration of a hematologic malignancy patient is increased by at least 0.040×10¹²/L after being administered oxygen reduced blood. In an aspect, the reticulocyte concentration of a hematologic malignancy patient is increased by at least 0.050×10¹²/L after being administered oxygen reduced blood. In an aspect, the reticulocyte concentration of a hematologic malignancy patient is increased by between 0.005×10¹²/L to 0.020×10¹²/L after being administered oxygen reduced blood. In an aspect, the reticulocyte concentration of a hematologic malignancy patient is increased by between 0.010×10¹²/L to 0.020×10¹²/L after being administered oxygen reduced blood. In an aspect, the reticulocyte concentration of a hematologic malignancy patient is increased by between 0.010×10¹²/L to 0.030×10¹²/L after being administered oxygen reduced blood. In an aspect, the reticulocyte concentration of a hematologic malignancy patient is increased by between 0.010×10¹²/L to 0.050×10¹²/L after being administered oxygen reduced blood. In an aspect, the reticulocyte concentration of a hematologic malignancy patient is increased by between 0.010×10¹²/L to 0.075×10¹²/L after being administered oxygen reduced blood. In an aspect, oxygen reduced blood is oxygen and carbon dioxide reduced blood.

In an aspect, the reticulocyte concentration of a hematologic malignancy patient is increased by at least 5%, at least 10%, at least 15%, at least 20%, at least 25%, or at least 30% after being administered oxygen reduced blood. In an aspect, the reticulocyte concentration of a hematologic malignancy patient is increased by at least 1-fold, at least 2-fold, at least 3-fold, at least 4-fold, or at least 5-fold after being administered oxygen reduced blood. In an aspect, the reticulocyte concentration of a hematologic malignancy patient is increased by at least 5% after being administered oxygen reduced blood compared to a hematologic malignancy patient having been administered the same volume of oxygen reduced blood. In an aspect, the reticulocyte concentration of a hematologic malignancy patient is increased by at least 10% after being administered oxygen reduced blood compared to a hematologic malignancy patient having been administered the same volume of oxygen reduced blood. In an aspect, the reticulocyte concentration of a hematologic malignancy patient is increased by at least 15% after being administered oxygen reduced blood compared to a hematologic malignancy patient having been administered the same volume of oxygen reduced blood. In an aspect, the reticulocyte concentration of a hematologic malignancy patient is increased by at least 20% after being administered oxygen reduced blood compared to a hematologic malignancy patient having been administered the same volume of oxygen reduced blood. In an aspect, the reticulocyte concentration of a hematologic malignancy patient is increased by at least 25% after being administered oxygen reduced blood compared to a hematologic malignancy patient having been administered the same volume of oxygen reduced blood. In an aspect, the reticulocyte concentration of a hematologic malignancy patient is increased by at least 30% after being administered oxygen reduced blood compared to a hematologic malignancy patient having been administered the same volume of oxygen reduced blood. In an aspect, the reticulocyte concentration of a hematologic malignancy patient is increased by at least 1-fold after being administered oxygen reduced blood compared to a hematologic malignancy patient having been administered the same volume of oxygen reduced blood. In an aspect, the reticulocyte concentration of a hematologic malignancy patient is increased by at least 2-fold after being administered oxygen reduced blood compared to a hematologic malignancy patient having been administered the same volume of oxygen reduced blood. In an aspect, the reticulocyte concentration of a hematologic malignancy patient is increased by at least 3-fold after being administered oxygen reduced blood compared to a hematologic malignancy patient having been administered the same volume of oxygen reduced blood. In an aspect, the reticulocyte concentration of a hematologic malignancy patient is increased by at least 4-fold after being administered oxygen reduced blood compared to a hematologic malignancy patient having been administered the same volume of oxygen reduced blood. In an aspect, the reticulocyte concentration of a hematologic malignancy patient is increased by at least 5-fold after being administered oxygen reduced blood compared to a hematologic malignancy patient having been administered the same volume of oxygen reduced blood. In an aspect, oxygen reduced blood is oxygen and carbon dioxide reduced blood.

In an aspect of the present disclosure, the monocyte concentration of a hematologic malignancy patient is less than 0.20×10⁹/L prior to being administered oxygen reduced blood. In an aspect, the monocyte concentration of a hematologic malignancy patient is less than 0.30×10⁹/L prior to being administered oxygen reduced blood. In an aspect, the monocyte concentration of a hematologic malignancy patient is less than 0.40×10⁹/L prior to being administered oxygen reduced blood. In an aspect, the monocyte concentration of a hematologic malignancy patient is less than 0.50×10⁹/L prior to being administered oxygen reduced blood. In an aspect, the monocyte concentration of a hematologic malignancy patient is less than 0.60×10⁹/L prior to being administered oxygen reduced blood. In an aspect, the monocyte concentration of a hematologic malignancy patient is between 0.20×10⁹/L to 0.30×10⁹/L prior to being administered oxygen reduced blood. In an aspect, the monocyte concentration of a hematologic malignancy patient is between 0.20×10⁹/L to 0.40×10⁹/L prior to being administered oxygen reduced blood. In an aspect, the monocyte concentration of a hematologic malignancy patient is between 0.20×10⁹/L to 0.50×10⁹/L prior to being administered oxygen reduced blood. In an aspect, the monocyte concentration of a hematologic malignancy patient is between 0.20×10⁹/L to 0.60×10⁹/L prior to being administered oxygen reduced blood. In an aspect, the monocyte concentration of a hematologic malignancy patient is between 0.30×10⁹/L to 0.50×10⁹/L prior to being administered oxygen reduced blood. In an aspect, the monocyte concentration of a hematologic malignancy patient is between 0.30×10⁹/L to 0.60×10⁹/L prior to being administered oxygen reduced blood. In an aspect, oxygen reduced blood is oxygen and carbon dioxide reduced blood.

In an aspect of the present disclosure, the monocyte concentration of a hematologic malignancy patient is 0.30×10⁹/L or more after being administered oxygen reduced blood. In an aspect, the monocyte concentration of the hematologic malignancy patient is 0.40×10⁹/L or more after being administered oxygen reduced blood. In an aspect, the monocyte concentration of the hematologic malignancy patient is 0.50×10⁹/L or more after being administered oxygen reduced blood. In an aspect, the monocyte concentration of the hematologic malignancy patient is 0.60×10⁹/L or more after being administered oxygen reduced blood. In an aspect, the monocyte concentration of the hematologic malignancy patient is 0.70×10⁹/L or more after being administered oxygen reduced blood. In an aspect, the monocyte concentration of the hematologic malignancy patient is 0.80×10⁹/L or more after being administered oxygen reduced blood. In an aspect, the monocyte concentration of a hematologic malignancy patient is between 0.30×10⁹/L to 0.50×10⁹/L after being administered oxygen reduced blood. In an aspect, the monocyte concentration of a hematologic malignancy patient is between 0.50×10⁹/L to 0.75×10⁹/L after being administered oxygen reduced blood. In an aspect, the monocyte concentration of a hematologic malignancy patient is between 0.30×10⁹/L to 0.090×10⁹/L after being administered oxygen reduced blood. In an aspect, a hematologic malignancy patient has a monocyte concentration that is within normal range after being administered oxygen reduced blood. In an aspect, oxygen reduced blood is oxygen and carbon dioxide reduced blood.

In an aspect of the present disclosure, the monocyte concentration of a hematologic malignancy patient is increased by at least 0.05×10⁹/L after being administered oxygen reduced blood. In an aspect, the monocyte concentration of a hematologic malignancy patient is increased by at least 0.10×10⁹/L after being administered oxygen reduced blood. In an aspect, the monocyte concentration of a hematologic malignancy patient is increased by at least 0.15×10⁹/L after being administered oxygen reduced blood. In an aspect, the monocyte concentration of a hematologic malignancy patient is increased by at least 0.20×10⁹/L after being administered oxygen reduced blood. In an aspect, the monocyte concentration of a hematologic malignancy patient is increased by at least 0.30×10⁹/L after being administered oxygen reduced blood. In an aspect, the monocyte concentration of a hematologic malignancy patient is increased by at least 0.40×10⁹/L after being administered oxygen reduced blood. In an aspect, the monocyte concentration of a hematologic malignancy patient is increased by at least 0.50×10⁹/L after being administered oxygen reduced blood. In an aspect, the monocyte concentration of a hematologic malignancy patient is increased by between 0.05×10⁹/L to 0.10×10⁹/L after being administered oxygen reduced blood. In an aspect, the monocyte concentration of a hematologic malignancy patient is increased by between 0.05×10⁹/L to 0.20×10⁹/L after being administered oxygen reduced blood. In an aspect, the monocyte concentration of a hematologic malignancy patient is increased by between 0.10×10⁹/L to 0.20×10⁹/L after being administered oxygen reduced blood. In an aspect, the monocyte concentration of a hematologic malignancy patient is increased by between 0.10×10⁹/L to 0.30×10⁹/L after being administered oxygen reduced blood. In an aspect, the monocyte concentration of a hematologic malignancy patient is increased by between 0.20×10⁹/L to 0.50×10⁹/L after being administered oxygen reduced blood. In an aspect, the monocyte concentration of a hematologic malignancy patient is increased by between 0.10×10⁹/L to 0.50×10⁹/L after being administered oxygen reduced blood. In an aspect, the monocyte concentration of a hematologic malignancy patient is increased by between 0.05×10⁹/L to 0.50×10⁹/L after being administered oxygen reduced blood. In an aspect, oxygen reduced blood is oxygen and carbon dioxide reduced blood.

In an aspect, the monocyte concentration of a hematologic malignancy patient is increased by at least 5%, at least 10%, at least 15%, at least 20%, at least 25%, or at least 30% after being administered oxygen reduced blood. In an aspect, the monocyte concentration of a hematologic malignancy patient is increased by at least 1-fold, at least 2-fold, at least 3-fold, at least 4-fold, or at least 5-fold after being administered oxygen reduced blood. In an aspect, the monocyte concentration of a hematologic malignancy patient is increased by at least 5% after being administered oxygen reduced blood compared to a hematologic malignancy patient having been administered the same volume of oxygen reduced blood. In an aspect, the monocyte concentration of a hematologic malignancy patient is increased by at least 10% after being administered oxygen reduced blood compared to a hematologic malignancy patient having been administered the same volume of oxygen reduced blood. In an aspect, the monocyte concentration of a hematologic malignancy patient is increased by at least 15% after being administered oxygen reduced blood compared to a hematologic malignancy patient having been administered the same volume of oxygen reduced blood. In an aspect, the monocyte concentration of a hematologic malignancy patient is increased by at least 20% after being administered oxygen reduced blood compared to a hematologic malignancy patient having been administered the same volume of oxygen reduced blood. In an aspect, the monocyte concentration of a hematologic malignancy patient is increased by at least 25% after being administered oxygen reduced blood compared to a hematologic malignancy patient having been administered the same volume of oxygen reduced blood. In an aspect, the monocyte concentration of a hematologic malignancy patient is increased by at least 30% after being administered oxygen reduced blood compared to a hematologic malignancy patient having been administered the same volume of oxygen reduced blood. In an aspect, the monocyte concentration of a hematologic malignancy patient is increased by at least 1-fold after being administered oxygen reduced blood compared to a hematologic malignancy patient having been administered the same volume of oxygen reduced blood. In an aspect, the monocyte concentration of a hematologic malignancy patient is increased by at least 2-fold after being administered oxygen reduced blood compared to a hematologic malignancy patient having been administered the same volume of oxygen reduced blood. In an aspect, the monocyte concentration of a hematologic malignancy patient is increased by at least 3-fold after being administered oxygen reduced blood compared to a hematologic malignancy patient having been administered the same volume of oxygen reduced blood. In an aspect, the monocyte concentration of a hematologic malignancy patient is increased by at least 4-fold after being administered oxygen reduced blood compared to a hematologic malignancy patient having been administered the same volume of oxygen reduced blood. In an aspect, the monocyte concentration of a hematologic malignancy patient is increased by at least 5-fold after being administered oxygen reduced blood compared to a hematologic malignancy patient having been administered the same volume of oxygen reduced blood. In an aspect, oxygen reduced blood is oxygen and carbon dioxide reduced blood.

Mean corpuscular volume (MCV) is a measure of the average volume of a red blood cell (corpuscle). MCV is calculated by according to the following formula

$$MCV = \frac{Hct \times 10}{[ RBC ]} ,$$

where Hct is the hematocrit of the patient's blood and [RBC] is the concentration of red blood cells in the patient's blood. MCV is typically expressed in femtoliters (fL, or 10⁻¹⁵ L), and a normal MCV range for a human is between 82 fL to 98 fL. In an aspect of the present disclosure, blood of a hematologic malignancy patient has an MCV of greater than 95 fL prior to being administered oxygen reduced blood. In an aspect, blood of a hematologic malignancy patient has an MCV of greater than 98 fL prior to being administered oxygen reduced blood. In an aspect, blood of a hematologic malignancy patient has an MCV of greater than 100 fL prior to being administered oxygen reduced blood. In an aspect, blood of a hematologic malignancy patient has an MCV of greater than 105 fL prior to being administered oxygen reduced blood. In an aspect, blood of a hematologic malignancy patient has an MCV of between 95 fL to 100 fL prior to being administered oxygen reduced blood. In an aspect, blood of a hematologic malignancy patient has an MCV of between 95 fL to 105 fL prior to being administered oxygen reduced blood. In an aspect, blood of a hematologic malignancy patient has an MCV of between 95 fL to 110 fL prior to being administered oxygen reduced blood. In an aspect, blood of a hematologic malignancy patient has an MCV of between 98 fL to 105 fL prior to being administered oxygen reduced blood. In an aspect, blood of a hematologic malignancy patient has an MCV of between 95 fL to 110 fL prior to being administered oxygen reduced blood. In an aspect, blood of a hematologic malignancy patient has an MCV of between 100 fL to 110 fL prior to being administered oxygen reduced blood. In an aspect, oxygen reduced blood is oxygen and carbon dioxide reduced blood.

In an aspect of the present disclosure, blood of a hematologic malignancy patient has an MCV of 100 fL or less after being administered oxygen reduced blood. In an aspect, blood of a hematologic malignancy patient has an MCV of 98 fL or less after being administered oxygen reduced blood. In an aspect, blood of a hematologic malignancy patient has an MCV of 95 fL or less after being administered oxygen reduced blood. In an aspect, blood of a hematologic malignancy patient has an MCV of 90 fL or less after being administered oxygen reduced blood. In an aspect, blood of a hematologic malignancy patient has an MCV of 85 fL or less after being administered oxygen reduced blood. In an aspect, blood of a hematologic malignancy patient has an MCV of between 95 fL to 98 fL after being administered oxygen reduced blood. In an aspect, blood of a hematologic malignancy patient has an MCV of between 95 fL to 100 fL after being administered oxygen reduced blood. In an aspect, blood of a hematologic malignancy patient has an MCV of between 90 fL to 95 fL after being administered oxygen reduced blood. In an aspect, blood of a hematologic malignancy patient has an MCV of between 90 fL to 98 fL after being administered oxygen reduced blood. In an aspect, blood of a hematologic malignancy patient has an MCV of between 90 fL to 100 fL after being administered oxygen reduced blood. In an aspect, blood of a hematologic malignancy patient has an MCV of between 85 fL to 90 fL after being administered oxygen reduced blood. In an aspect, blood of a hematologic malignancy patient has an MCV of between 85 fL to 95 fL after being administered oxygen reduced blood. In an aspect, blood of a hematologic malignancy patient has an MCV of between 85 fL to 98 fL after being administered oxygen reduced blood. In an aspect, blood of a hematologic malignancy patient has an MCV of between 85 fL to 100 fL after being administered oxygen reduced blood. In an aspect, blood of a hematologic malignancy patient has an MCV of between 80 fL to 90 fL after being administered oxygen reduced blood. In an aspect, blood of a hematologic malignancy patient has an MCV of between 80 fL to 95 fL after being administered oxygen reduced blood. In an aspect, blood of a hematologic malignancy patient has an MCV of between 80 fL to 98 fL after being administered oxygen reduced blood. In an aspect, blood of a hematologic malignancy patient has an MCV that is within normal range after being administered oxygen reduced blood. In an aspect, oxygen reduced blood is oxygen and carbon dioxide reduced blood.

In an aspect of the present disclosure, the MCV of blood of a hematologic malignancy patient is reduced by at least 1 fL after being administered oxygen reduced blood. In an aspect, the MCV of blood of a hematologic malignancy patient is reduced by at least 2 fL after being administered oxygen reduced blood. In an aspect, the MCV of blood of a hematologic malignancy patient is reduced by at least 3 fL after being administered oxygen reduced blood. In an aspect, the MCV of blood of a hematologic malignancy patient is reduced by at least 5 fL after being administered oxygen reduced blood. In an aspect, the MCV of blood of a hematologic malignancy patient is reduced by at least 10 fL after being administered oxygen reduced blood. In an aspect, the MCV of blood of a hematologic malignancy patient is reduced by at least 15 fL after being administered oxygen reduced blood. In an aspect, the MCV of blood of a hematologic malignancy patient is reduced by between 1 fL to 5 fL after being administered oxygen reduced blood. In an aspect, the MCV of blood of a hematologic malignancy patient is reduced by between 1 fL to 10 fL after being administered oxygen reduced blood. In an aspect, the MCV of blood of a hematologic malignancy patient is reduced by between 1 fL to 15 fL after being administered oxygen reduced blood. In an aspect, the MCV of blood of a hematologic malignancy patient is reduced by between 1 fL to 20 fL after being administered oxygen reduced blood. In an aspect, the MCV of blood of a hematologic malignancy patient is reduced by between 2 fL to 5 fL after being administered oxygen reduced blood. In an aspect, the MCV of blood of a hematologic malignancy patient is reduced by between 2 fL to 10 fL after being administered oxygen reduced blood. In an aspect, the MCV of blood of a hematologic malignancy patient is reduced by between 2 fL to 15 fL after being administered oxygen reduced blood. In an aspect, the MCV of blood of a hematologic malignancy patient is reduced by between 2 fL to 20 fL after being administered oxygen reduced blood. In an aspect, the MCV of blood of a hematologic malignancy patient is reduced by between 5 fL to 10 fL after being administered oxygen reduced blood. In an aspect, the MCV of blood of a hematologic malignancy patient is reduced by between 5 fL to 15 fL after being administered oxygen reduced blood. In an aspect, the MCV of blood of a hematologic malignancy patient is reduced by between 5 fL to 20 fL after being administered oxygen reduced blood. In an aspect, the MCV of blood of a hematologic malignancy patient is reduced by between 10 fL to 15 fL after being administered oxygen reduced blood. In an aspect, the MCV of blood of a hematologic malignancy patient is reduced by between 10 fL to 20 fL after being administered oxygen reduced blood. In an aspect, oxygen reduced blood is oxygen and carbon dioxide reduced blood.

In an aspect of the present disclosure, the MCV of blood of a hematologic malignancy patient is reduced by at least 5%, at least 10%, at least 15%, at least 20%, at least 25%, or at least 30% after being administered oxygen reduced blood. In an aspect, the MCV of blood of a hematologic malignancy patient is reduced by at least 1-fold, at least 2-fold, at least 3-fold, at least 4-fold, or at least 5-fold after being administered oxygen reduced blood. In an aspect, the MCV of blood of a hematologic malignancy patient is reduced by at least 5% after being administered oxygen reduced blood compared to a hematologic malignancy patient having been administered the same volume of oxygen reduced blood. In an aspect, the MCV of blood of a hematologic malignancy patient is reduced by at least 10% after being administered oxygen reduced blood compared to a hematologic malignancy patient having been administered the same volume of oxygen reduced blood. In an aspect, the MCV of blood of a hematologic malignancy patient is reduced by at least 15% after being administered oxygen reduced blood compared to a hematologic malignancy patient having been administered the same volume of oxygen reduced blood. In an aspect, the MCV of blood of a hematologic malignancy patient is reduced by at least 20% after being administered oxygen reduced blood compared to a hematologic malignancy patient having been administered the same volume of oxygen reduced blood. In an aspect, the MCV of blood of a hematologic malignancy patient is reduced by at least 25% after being administered oxygen reduced blood compared to a hematologic malignancy patient having been administered the same volume of oxygen reduced blood. In an aspect, the MCV of blood of a hematologic malignancy patient is reduced by at least 30% after being administered oxygen reduced blood compared to a hematologic malignancy patient having been administered the same volume of oxygen reduced blood. In an aspect, the MCV of blood of a hematologic malignancy patient is reduced by at least 1-fold after being administered oxygen reduced blood compared to a hematologic malignancy patient having been administered the same volume of oxygen reduced blood. In an aspect, the MCV of blood of a hematologic malignancy patient is reduced by at least 2-fold after being administered oxygen reduced blood compared to a hematologic malignancy patient having been administered the same volume of oxygen reduced blood. In an aspect, the MCV of blood of a hematologic malignancy patient is reduced by at least 3-fold after being administered oxygen reduced blood compared to a hematologic malignancy patient having been administered the same volume of oxygen reduced blood. In an aspect, the MCV of blood of a hematologic malignancy patient is reduced by at least 4-fold after being administered oxygen reduced blood compared to a hematologic malignancy patient having been administered the same volume of oxygen reduced blood. In an aspect, the MCV of blood of a hematologic malignancy patient is reduced by at least 5-fold after being administered oxygen reduced blood compared to a hematologic malignancy patient having been administered the same volume of oxygen reduced blood. In an aspect, oxygen reduced blood is oxygen and carbon dioxide reduced blood.

Methods of the present disclosure provide for, and include, preventing the occurrence of a transfusion-related adverse event in a hematologic malignancy patient in need thereof comprising administering at least two units of oxygen reduced blood to the hematologic malignancy patient. Methods of the present disclosure provide for, and include, reducing the occurrence of a transfusion-related adverse event in a hematologic malignancy patient in need thereof comprising administering at least two units of oxygen reduced blood to the hematologic malignancy patient. Methods of the present disclosure provide for, and include, preventing or reducing the occurrence of a transfusion-related adverse event in a hematologic malignancy patient in need thereof comprising administering at least two units of oxygen reduced blood to the hematologic malignancy patient. As used herein, an adverse event is an unfavorable event resulting from a blood transfusion. In an aspect of the present disclosure, administering oxygen reduced blood to a hematologic malignancy patient prevents a transfusion-related adverse event for at least 7 days, at least 14 days, at least 21 days, at least 28 days, at least 35 days, or at least 48 days after the administration. In an aspect, administering oxygen reduced blood to a hematologic malignancy patient reduces a transfusion-related adverse event for at least 7 days, at least 14 days, at least 21 days, at least 28 days, at least 35 days, or at least 48 days after the administration. In an aspect, administering oxygen reduced blood to a hematologic malignancy patient prevents or reduces a transfusion-related adverse event for at least 7 days, at least 14 days, at least 21 days, at least 28 days, at least 35 days, or at least 48 days after the administration. In an aspect, no transfusion-related adverse event occurs for at least 7 days after administering oxygen reduced blood to the hematologic malignancy patient. In an aspect, no transfusion-related adverse event occurs for at least 14 days after administering oxygen reduced blood to the hematologic malignancy patient. In an aspect, no transfusion-related adverse event occurs for at least 21 days after administering oxygen reduced blood to the hematologic malignancy patient. In an aspect, no transfusion-related adverse event occurs for at least 28 days after administering oxygen reduced blood to the hematologic malignancy patient. In an aspect, no transfusion-related adverse event occurs for at least 35 days after administering oxygen reduced blood to the hematologic malignancy patient. In an aspect, a transfusion-related adverse event is reduced for at least 7 days after administering oxygen reduced blood to the hematologic malignancy patient. In an aspect, a transfusion-related adverse event is reduced for at least 14 days after administering oxygen reduced blood to the hematologic malignancy patient. In an aspect, a transfusion-related adverse event is reduced for at least 21 days after administering oxygen reduced blood to the hematologic malignancy patient. In an aspect, In an aspect, a transfusion-related adverse event is reduced for at least 28 days after administering oxygen reduced blood to the hematologic malignancy patient. In an aspect, a transfusion-related adverse event is reduced for at least 35 days after administering oxygen reduced blood to the hematologic malignancy patient.

In an aspect of the present disclosure, a transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), hematoma, arterial puncture, delayed bleeding, localized infection or inflammation, brachial artery pseudoaneurysm, deep vein thrombosis (DVT), vasovagal reactions, nerve injury or irritation, compartment syndrome, arteriovenous fistula, and any combination thereof. In an aspect, the transfusion-related adverse event is iron overload. In an aspect, the transfusion-related adverse event is hemorrhagic shock. In an aspect, the transfusion-related adverse event is alloimmunization. In an aspect, the transfusion-related adverse event is an allergic reaction. In an aspect, the transfusion-related adverse event is a transfusion-transmitted infection (TTI). In an aspect, the transfusion-related adverse event is transfusion-associated graft versus host disease. In an aspect, the transfusion-related adverse event is transfusion-related acute lung injury (TRALI). In an aspect, the transfusion-related adverse event is post-transfusion purpura. In an aspect, the transfusion-related adverse event is transfusion-associated circulatory overload (TACO). In an aspect, the transfusion-related adverse event is transfusion-associated dyspnea (TAD). In an aspect, the transfusion-related adverse event is febrile non-hemolytic transfusion reaction (FNHTR). In an aspect, the transfusion-related adverse event is hypotensive transfusion reaction. In an aspect, the transfusion-related adverse event is delayed hemolytic transfusion reaction (DHTR). In an aspect, the transfusion-related adverse event is delayed serologic transfusion reaction (DSTR). In an aspect, the transfusion-related adverse event is acute hemolytic transfusion reaction (AHTR). In an aspect, the transfusion-related adverse event is a major cardiac event (MCE). In an aspect, the transfusion-related adverse event is hematoma. In an aspect, the transfusion-related adverse event is arterial puncture. In an aspect, the transfusion-related adverse event is delayed bleeding. In an aspect, the transfusion-related adverse event is localized infection or inflammation. In an aspect, the transfusion-related adverse event is brachial artery pseudoaneurysm. In an aspect, the transfusion-related adverse event is deep vein thrombosis (DVT). In an aspect, the transfusion-related adverse event is vasovagal reactions. In an aspect, the transfusion-related adverse event is nerve injury or irritation. In an aspect, the transfusion-related adverse event is compartment syndrome. In an aspect, the transfusion-related adverse event is arteriovenous fistula. In an aspect, a transfusion-related adverse event is a combination of 1, 2, 3, 4, 5, 6, 7, 8, 9, 10, 11, or 12 transfusion-related adverse events.

In an aspect, a transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death. In an aspect, the transfusion-related serious adverse event is a life-threatening illness. In an aspect, the transfusion-related serious adverse event is a life-threatening injury. In an aspect, the transfusion-related serious adverse event is permanent impairment of a body structure. In an aspect, the transfusion-related serious adverse event is permanent impairment of a body function. In an aspect, the transfusion-related serious adverse event is hospitalization. In an aspect, the transfusion-related serious adverse event is prolongation of hospitalization. In an aspect, the transfusion-related serious adverse event is a need for medical or surgical intervention to prevent a life-threatening illness or injury. In an aspect, the transfusion-related serious adverse event is contraction of a chronic disease. In an aspect, the transfusion-related serious adverse event is death. In an aspect, a transfusion-related adverse event is a combination of 1, 2, 3, 4, 5, 6, or 7 serious adverse events.

Methods of the present disclosure provide for, and include, increasing the blood transfusion interval for a hematologic malignancy patient in need thereof by administering at least two units of oxygen reduced blood, as provided herein, to the hematologic malignancy patient. As used herein, an increased blood transfusion interval means an increase in the period of time between subsequent blood transfusions (i.e., resulting from less frequent blood transfusions administered over time). As used herein, an average interval between blood transfusions refers to the average (mean) time between blood transfusions for a series of two or more blood transfusions. In an aspect, the average interval between subsequent blood transfusions administered to the hematologic malignancy patient is increased by at least 5% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by at least 10% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by at least 15% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by at least 20% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by at least 25% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by at least 30% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by at least 35% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by at least 40% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by at least 45% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by at least 50% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between 10% to 20% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between 10% to 30% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between 10% to 40% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between 10% to 50% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between 20% to 30% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between 20% to 40% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between 20% to 50% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between 30% to 40% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between 30% to 50% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between 5% to 50% compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased at least one-fold compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased at least two-fold compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased at least three-fold compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased at least four-fold compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased at least five-fold compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by about one week compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by about two weeks compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by about three weeks compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by about four weeks compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between one day and one week compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between one week and two weeks compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between one week and four weeks compared to the average interval between blood transfusions administered to a hematologic malignancy patient previously transfused with an identical volume of non-oxygen reduced blood. In an aspect, the average interval between subsequent blood transfusions administered to the hematologic malignancy patient is increased by between two weeks and four weeks compared to the average interval between blood transfusions administered to a hematologic malignancy patient previously transfused with an identical volume of non-oxygen reduced blood.

In an aspect of the present disclosure, the average interval between blood transfusions administered to a hematologic malignancy patient is increased by at least 5% after administering at least two units of oxygen reduced blood, as provided herein, to a hematologic malignancy patient. In an aspect, the average interval between blood transfusions administered to a hematologic malignancy patient is increased by at least 10% after administering at least two units of oxygen reduced blood, as provided herein, to a hematologic malignancy patient. In an aspect, the average interval between blood transfusions administered to a hematologic malignancy patient is increased by at least 15% after administering at least two units of oxygen reduced blood, as provided herein, to a hematologic malignancy patient. In an aspect, the average interval between blood transfusions administered to a hematologic malignancy patient is increased by at least 20% after administering at least two units of oxygen reduced blood, as provided herein, to a hematologic malignancy patient. In an aspect, the average interval between blood transfusions administered to a hematologic malignancy patient is increased by at least 25% after administering at least two units of oxygen reduced blood, as provided herein, to a hematologic malignancy patient. In an aspect, the average interval between blood transfusions administered to a hematologic malignancy patient is increased by at least 25% after administering at least two units of oxygen reduced blood, as provided herein, to a hematologic malignancy patient. In an aspect, the average interval between blood transfusions administered to a hematologic malignancy patient is increased by at least 30% after administering at least two units of oxygen reduced blood, as provided herein, to a hematologic malignancy patient. In an aspect, the average interval between blood transfusions administered to a hematologic malignancy patient is increased by at least 35% after administering at least two units of oxygen reduced blood, as provided herein, to a hematologic malignancy patient. In an aspect, the average interval between blood transfusions administered to a hematologic malignancy patient is increased by at least 40% after administering at least two units of oxygen reduced blood, as provided herein, to a hematologic malignancy patient. In an aspect, the average interval between blood transfusions administered to a hematologic malignancy patient is increased by at least 45% after administering at least two units of oxygen reduced blood, as provided herein, to a hematologic malignancy patient. In an aspect, the average interval between blood transfusions administered to a hematologic malignancy patient is increased by at least 50% after administering at least two units of oxygen reduced blood, as provided herein, to a hematologic malignancy patient. In an aspect, the average interval between blood transfusions administered to a hematologic malignancy patient is increased at least one-fold after administering at least two units of oxygen reduced blood, as provided herein, to a hematologic malignancy patient. In an aspect, the average interval between blood transfusions administered to a hematologic malignancy patient is increased at least two-fold after administering at least two units of oxygen reduced blood, as provided herein, to a hematologic malignancy patient. In an aspect, the average interval between blood transfusions administered to a hematologic malignancy patient is increased at least three-fold after administering at least two units of oxygen reduced blood, as provided herein, to a hematologic malignancy patient. In an aspect, the average interval between blood transfusions administered to a hematologic malignancy patient is increased at least four-fold after administering at least two units of oxygen reduced blood, as provided herein, to a hematologic malignancy patient. In an aspect, the average interval between blood transfusions administered to a hematologic malignancy patient is increased at least five-fold after administering at least two units of oxygen reduced blood, as provided herein, to a hematologic malignancy patient. In an aspect, the average interval between blood transfusions administered to a hematologic malignancy patient is increased by about one day after administering at least two units of oxygen reduced blood, as provided herein, to a hematologic malignancy patient. In an aspect, the average interval between blood transfusions administered to a hematologic malignancy patient is increased by about two days after administering at least two units of oxygen reduced blood, as provided herein, to a hematologic malignancy patient. In an aspect, the average interval between blood transfusions administered to a hematologic malignancy patient is increased by about three days after administering at least two units of oxygen reduced blood, as provided herein, to a hematologic malignancy patient. In an aspect, the average interval between blood transfusions administered to a hematologic malignancy patient is increased by about four days after administering at least two units of oxygen reduced blood, as provided herein, to a hematologic malignancy patient. In an aspect, the average interval between blood transfusions administered to a hematologic malignancy patient is increased by about five days after administering at least two units of oxygen reduced blood, as provided herein, to a hematologic malignancy patient. In an aspect, the average interval between blood transfusions administered to a hematologic malignancy patient is increased by about six days after administering at least two units of oxygen reduced blood, as provided herein, to a hematologic malignancy patient. In an aspect, the average interval between blood transfusions administered to a hematologic malignancy patient is increased by about one week after administering at least two units of oxygen reduced blood, as provided herein, to a hematologic malignancy patient. In an aspect, the average interval between blood transfusions administered to a hematologic malignancy patient is increased by about two weeks after administering at least two units of oxygen reduced blood, as provided herein, to a hematologic malignancy patient. In an aspect, the average interval between blood transfusions administered to a hematologic malignancy patient is increased by about three weeks after administering at least two units of oxygen reduced blood, as provided herein, to a hematologic malignancy patient. In an aspect, the average interval between blood transfusions administered to a hematologic malignancy patient is increased by about four weeks after administering at least two units of oxygen reduced blood, as provided herein, to a hematologic malignancy patient. In an aspect, the average interval between blood transfusions administered to a hematologic malignancy patient is increased by between one day and one week after administering at least two units of oxygen reduced blood, as provided herein, to a hematologic malignancy patient. In an aspect, the average interval between blood transfusions administered to a hematologic malignancy patient is increased by between one week and two weeks after administering at least two units of oxygen reduced blood, as provided herein, to a hematologic malignancy patient. In an aspect, the average interval between blood transfusions administered to a hematologic malignancy patient is increased by between one day to one week after administering at least two units of oxygen reduced blood, as provided herein, to a hematologic malignancy patient. In an aspect, the average interval between blood transfusions administered to a hematologic malignancy patient is increased by between one week to two weeks after administering at least two units of oxygen reduced blood, as provided herein, to a hematologic malignancy patient. In an aspect, the average interval between blood transfusions administered to a hematologic malignancy patient is increased by between one week and four weeks after administering at least two units of oxygen reduced blood, as provided herein, to a hematologic malignancy patient. In an aspect, the average interval between blood transfusions administered to a hematologic malignancy patient is increased by between two weeks to four weeks after administering at least two units of oxygen reduced blood, as provided herein, to a hematologic malignancy patient.

Methods of the present disclosure provide for, and include, reducing the blood transfusion volume for a hematologic malignancy patient in need thereof by administering at least two units of oxygen reduced blood, as provided herein, to the hematologic malignancy patient. As used herein, a reduced blood transfusion volume is a reduction in the volume of blood administered after an initial administration. As used herein, an average interval between blood transfusions refers to the average (mean) volume of a series of two or more blood transfusions. In an aspect, the average blood transfusion volume of subsequent blood transfusions administered to a hematologic malignancy patient is reduced by at least 5% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least 10% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least 15% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least 20% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least 25% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least 30% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least 35% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least 40% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least 45% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least 50% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by between 10% to 20% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by between 10% to 30% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by between 10% to 40% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by between 10% to 50% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by between 20% to 30% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by between 20% to 40% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by between 20% to 50% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by between 30% to 40% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by between 30% to 50% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by between 5% to 50% compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least one-fold compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least two-fold compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least three-fold compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least four-fold compared to the average blood transfusion volume administered to a hematologic malignancy patient previously transfused with an identical volume of non-oxygen reduced blood. In an aspect, the average blood transfusion volume of subsequent blood transfusions administered to a hematologic malignancy patient is reduced by at least five-fold compared to the average blood transfusion volume administered to a hematologic malignancy patient previously transfused with an identical volume of non-oxygen reduced blood.

In an aspect, the average transfusion volume of subsequent blood transfusions administered to a hematologic malignancy patient is 5% less than the initial volume of oxygen reduced blood administered to the patient. In an aspect, the average transfusion volume of subsequent blood transfusions administered to a hematologic malignancy patient is 10% less than the initial volume of oxygen reduced blood administered to the patient. In an aspect, the average transfusion volume of subsequent blood transfusions administered to a hematologic malignancy patient is 20% less than the initial volume of oxygen reduced blood administered to the patient. In an aspect, the average transfusion volume of subsequent blood transfusions administered to a hematologic malignancy patient is 30% less than the initial volume of oxygen reduced blood administered to the patient. In an aspect, the average transfusion volume of subsequent blood transfusions administered to a hematologic malignancy patient is 40% less than the initial volume of oxygen reduced blood administered to the patient. In an aspect, the average transfusion volume of subsequent blood transfusions administered to a hematologic malignancy patient is 50% less than the initial volume of oxygen reduced blood administered to the patient. In an aspect, the average transfusion volume of subsequent blood transfusions administered to a hematologic malignancy patient is between 10% to 20% less than the initial volume of oxygen reduced blood administered to the patient. In an aspect, the average transfusion volume of subsequent blood transfusions administered to a hematologic malignancy patient is between 10% to 30% less than the initial volume of oxygen reduced blood administered to the patient. In an aspect, the average transfusion volume of subsequent blood transfusions administered to a hematologic malignancy patient is between 10% to 40% less than the initial volume of oxygen reduced blood administered to the patient. In an aspect, the average transfusion volume of subsequent blood transfusions administered to a hematologic malignancy patient is between 10% to 50% less than the initial volume of oxygen reduced blood administered to the patient. In an aspect, the average transfusion volume of subsequent blood transfusions administered to a hematologic malignancy patient is between 20% to 30% less than the initial volume of oxygen reduced blood administered to the patient. In an aspect, the average transfusion volume of subsequent blood transfusions administered to a hematologic malignancy patient is between 20% to 40% less than the initial volume of oxygen reduced blood administered to the patient. In an aspect, the average transfusion volume of subsequent blood transfusions administered to a hematologic malignancy patient is between 20% to 50% less than the initial volume of oxygen reduced blood administered to the patient. In an aspect, the average transfusion volume of subsequent blood transfusions administered to a hematologic malignancy patient is between 30% to 40% less than the initial volume of oxygen reduced blood administered to the patient. In an aspect, the average transfusion volume of subsequent blood transfusions administered to a hematologic malignancy patient is between 30% to 50% less than the initial volume of oxygen reduced blood administered to the patient. In an aspect, the average transfusion volume of subsequent blood transfusions administered to a hematologic malignancy patient is between 5% to 50% less than the initial volume of oxygen reduced blood administered to the patient. In an aspect, the average transfusion volume of subsequent blood transfusions administered to a hematologic malignancy patient is at least one-fold less than the initial volume of oxygen reduced blood administered to the patient. In an aspect, the average transfusion volume of subsequent blood transfusions administered to a hematologic malignancy patient is at least two-fold less than the initial volume of oxygen reduced blood administered to the patient. In an aspect, the average transfusion volume of subsequent blood transfusions administered to a hematologic malignancy patient is at least three-fold less than the initial volume of oxygen reduced blood administered to the patient. In an aspect, the average transfusion volume of subsequent blood transfusions administered to a hematologic malignancy patient is at least four-fold less than the initial volume of oxygen reduced blood administered to the patient. In an aspect, the average transfusion volume of subsequent blood transfusions administered to a hematologic malignancy patient is at least five-fold less than the initial volume of oxygen reduced blood administered to the patient.

In an aspect, the average transfusion volume of subsequent blood transfusions administered to a hematologic malignancy patient having been previously transfused with an oxygen reduced blood is less than 1 liter (L). In an aspect, the average transfusion volume of subsequent blood transfusions administered to a hematologic malignancy patient having been previously transfused with an oxygen reduced blood is less than 900 milliliters (mL). In an aspect, the average transfusion volume of subsequent blood transfusions administered to a hematologic malignancy patient having been previously transfused with an oxygen reduced blood is less than 800 mL. In an aspect, the average transfusion volume of subsequent blood transfusions administered to a hematologic malignancy patient having been previously transfused with an oxygen reduced blood is less than 700 mL. In an aspect, the average transfusion volume of subsequent blood transfusions administered to a hematologic malignancy patient having been previously transfused with an oxygen reduced blood is less than 600 mL. In an aspect, the average transfusion volume of subsequent blood transfusions administered to a hematologic malignancy patient having been previously transfused with an oxygen reduced blood is less than 500 mL. In an aspect, the average transfusion volume of subsequent blood transfusions administered to a hematologic malignancy patient having been previously transfused with an oxygen reduced blood is less than 400 mL. In an aspect, the average transfusion volume of subsequent blood transfusions administered to a hematologic malignancy patient having been previously transfused with an oxygen reduced blood is less than 250 mL. In an aspect, the average transfusion volume of subsequent blood transfusions administered to a hematologic malignancy patient having been previously transfused with an oxygen reduced blood is between 500 mL and 1 L. In an aspect, the average transfusion volume of subsequent blood transfusions administered to a hematologic malignancy patient having been previously transfused with an oxygen reduced blood is 2 units or less. In an aspect, the average transfusion volume of subsequent blood transfusions administered to a hematologic malignancy patient having been previously transfused with an oxygen reduced blood is 2 units. In an aspect, the average transfusion volume of subsequent blood transfusions administered to a hematologic malignancy patient having been previously transfused with an oxygen reduced blood is 1.5 units or less. In an aspect, the average transfusion volume of subsequent blood transfusions administered to a hematologic malignancy patient having been previously transfused with an oxygen reduced blood is 1.5 units. In an aspect, the average transfusion volume of subsequent blood transfusions administered to a hematologic malignancy patient having been previously transfused with an oxygen reduced blood is 1 unit or less. In an aspect, the average transfusion volume of subsequent blood transfusions administered to a hematologic malignancy patient having been previously transfused with an oxygen reduced blood is 1 unit.

Methods of the present disclosure provide for, and include, improving oxygen delivery to a hematologic malignancy patient in need thereof comprising administering at least two units of oxygen reduced blood, as provided herein, to the hematologic malignancy patient. As used herein, oxygen delivery/oxygenation is measured by blood oxygen level. In an aspect, the blood oxygen level of the hematologic malignancy patient is at least 5% greater than the blood oxygen level of a hematologic malignancy patient transfused with an identical volume of non-oxygen reduced blood. In an aspect, the blood oxygen level of the hematologic malignancy patient is at least 10% greater than the blood oxygen level of a hematologic malignancy patient transfused with an identical volume of non-oxygen reduced blood. In an aspect, the blood oxygen level of the hematologic malignancy patient is at least 15% greater than the blood oxygen level of a hematologic malignancy patient transfused with an identical volume of non-oxygen reduced blood. In an aspect, the blood oxygen level of the hematologic malignancy patient is at least 20% greater than the blood oxygen level of a hematologic malignancy patient transfused with an identical volume of non-oxygen reduced blood. In an aspect, the blood oxygen level of the hematologic malignancy patient is at least 25% greater than the blood oxygen level of a hematologic malignancy patient transfused with an identical volume of non-oxygen reduced blood. In an aspect, the blood oxygen level of the hematologic malignancy patient is at least 30% greater than the blood oxygen level of a hematologic malignancy patient transfused with an identical volume of non-oxygen reduced blood. In an aspect, the blood oxygen level of the hematologic malignancy patient is at least 35% greater than the blood oxygen level of a hematologic malignancy patient transfused with an identical volume of non-oxygen reduced blood. In an aspect, the blood oxygen level of the hematologic malignancy patient is at least 40% greater than the blood oxygen level of a hematologic malignancy patient transfused with an identical volume of non-oxygen reduced blood. In an aspect, the blood oxygen level of the hematologic malignancy patient is at least 45% greater than the blood oxygen level of a hematologic malignancy patient transfused with an identical volume of non-oxygen reduced blood. In an aspect, the blood oxygen level of the hematologic malignancy patient is at least 50% greater than the blood oxygen level of a hematologic malignancy patient transfused with an identical volume of non-oxygen reduced blood. In an aspect, the blood oxygen level of the hematologic malignancy patient is between 10% to 20% greater than the blood oxygen level of a hematologic malignancy patient transfused with an identical volume of non-oxygen reduced blood. In an aspect, the blood oxygen level of the hematologic malignancy patient is between 10% to 30% greater than the blood oxygen level of a hematologic malignancy patient transfused with an identical volume of non-oxygen reduced blood. In an aspect, the blood oxygen level of the hematologic malignancy patient is between 10% to 40% greater than the blood oxygen level of a hematologic malignancy patient transfused with an identical volume of non-oxygen reduced blood. In an aspect, the blood oxygen level of the hematologic malignancy patient is between 10% to 50% greater than the blood oxygen level of a hematologic malignancy patient transfused with an identical volume of non-oxygen reduced blood. In an aspect, the blood oxygen level of the hematologic malignancy patient is between 20% to 30% greater than the blood oxygen level of a hematologic malignancy patient transfused with an identical volume of non-oxygen reduced blood. In an aspect, the blood oxygen level of the hematologic malignancy patient is between 20% to 40% greater than the blood oxygen level of a hematologic malignancy patient transfused with an identical volume of non-oxygen reduced blood. In an aspect, the blood oxygen level of the hematologic malignancy patient is between 20% to 50% greater than the blood oxygen level of a hematologic malignancy patient transfused with an identical volume of non-oxygen reduced blood. In an aspect, the blood oxygen level of the hematologic malignancy patient is between 30% to 40% greater than the blood oxygen level of a hematologic malignancy patient transfused with an identical volume of non-oxygen reduced blood. In an aspect, the blood oxygen level of the hematologic malignancy patient is between 30% to 50% greater than the blood oxygen level of a hematologic malignancy patient transfused with an identical volume of non-oxygen reduced blood. In an aspect, the blood oxygen level of the hematologic malignancy patient is between 5% to 50% greater than the blood oxygen level of a hematologic malignancy patient transfused with an identical volume of non-oxygen reduced blood.

In an aspect, the blood oxygen level of the hematologic malignancy patient is at least 5% higher than the blood oxygen level of the patient before oxygen reduced blood was administered. In an aspect, the blood oxygen level of the hematologic malignancy patient is at least 10% higher than the blood oxygen level of the patient before oxygen reduced blood was administered. In an aspect, the blood oxygen level of the hematologic malignancy patient is at least 20% higher than the blood oxygen level of the patient before oxygen reduced blood was administered. In an aspect, the blood oxygen level of the hematologic malignancy patient is at least 30% higher than the blood oxygen level of the patient before oxygen reduced blood was administered. In an aspect, the blood oxygen level of the hematologic malignancy patient is at least 40% higher than the blood oxygen level of the patient before oxygen reduced blood was administered. In an aspect, the blood oxygen level of the hematologic malignancy patient is at least 50% higher than the blood oxygen level of the patient before oxygen reduced blood was administered. In an aspect, the blood oxygen level of the hematologic malignancy patient is between 10% to 20% higher than the blood oxygen level of the patient before oxygen reduced blood was administered. In an aspect, the blood oxygen level of the hematologic malignancy patient is between 10% to 30% higher than the blood oxygen level of the patient before oxygen reduced blood was administered. In an aspect, the blood oxygen level of the hematologic malignancy patient is between 10% to 40% higher than the blood oxygen level of the patient before oxygen reduced blood was administered. In an aspect, the blood oxygen level of the hematologic malignancy patient is between 10% to 50% higher than the blood oxygen level of the patient before oxygen reduced blood was administered. In an aspect, the blood oxygen level of the hematologic malignancy patient is between 20% to 30% higher than the blood oxygen level of the patient before oxygen reduced blood was administered. In an aspect, the blood oxygen level of the hematologic malignancy patient is between 20% to 40% higher than the blood oxygen level of the patient before oxygen reduced blood was administered. In an aspect, the blood oxygen level of the hematologic malignancy patient is between 20% to 50% higher than the blood oxygen level of the patient before oxygen reduced blood was administered. In an aspect, the blood oxygen level of the hematologic malignancy patient is between 5% to 50% higher than the blood oxygen level of the patient before oxygen reduced blood was administered.

In an aspect, the blood oxygen level of the hematologic malignancy patient is less than 80% prior to being administered oxygen reduced blood. In an aspect, the blood oxygen level of the hematologic malignancy patient is less than 85% prior to being administered oxygen reduced blood. In an aspect, the blood oxygen level of the hematologic malignancy patient is less than 90% prior to being administered oxygen reduced blood. In an aspect, the blood oxygen level of the hematologic malignancy patient is between 80% to 85% prior to being administered oxygen reduced blood. In an aspect, the blood oxygen level of the hematologic malignancy patient is between 80% to 90% prior to being administered oxygen reduced blood. In an aspect, the blood oxygen level of the hematologic malignancy patient is at least 90% after being administered oxygen reduced blood. In an aspect, the blood oxygen level of the hematologic malignancy patient is at least 95% after being administered oxygen reduced blood. In an aspect, the blood oxygen level of the hematologic malignancy patient is between 90% to 100% after being administered oxygen reduced blood. In an aspect, the blood oxygen level of the hematologic malignancy patient is in normal range after administered oxygen reduced blood.

Methods of the present disclosure provide for, and include, preventing transfusion-related sequelae in a hematologic malignancy patient in need thereof by administering at least two units of oxygen reduced blood, as provided herein, to the hematologic malignancy patient. Methods of the present disclosure provide for, and include, reducing transfusion-related sequelae in a hematologic malignancy patient in need thereof by administering at least two units of oxygen reduced blood, as provided herein, to the hematologic malignancy patient. Methods of the present disclosure provide for, and include, preventing or reducing transfusion-related sequelae in a hematologic malignancy patient in need thereof by administering at least two units of oxygen reduced blood, as provided herein, to the hematologic malignancy patient. As used herein, transfusion-related sequelae are adverse events resulting from a blood transfusion. In an aspect, administering oxygen reduced blood to a hematologic malignancy patient prevents transfusion-related sequelae for at least 7 days, at least 14 days, at least 21 days, at least 28 days, at least 35 days, or at least 48 days after the administration. In an aspect, administering oxygen reduced blood to a hematologic malignancy patient reduces transfusion-related sequelae for at least 7 days, at least 14 days, at least 21 days, at least 28 days, at least 35 days, or at least 48 days after the administration. In an aspect, administering oxygen reduced blood to a hematologic malignancy patient prevents or reduces transfusion-related sequelae for at least 7 days, at least 14 days, at least 21 days, at least 28 days, at least 35 days, or at least 48 days after the administration. In an aspect, no transfusion-related sequelae occur for 7 days after administering oxygen reduced blood to the hematologic malignancy patient. In an aspect, one transfusion-related sequela occurs over a 7 day period after administering oxygen reduced blood to the hematologic malignancy patient. In an aspect, two transfusion-related sequelae occurs over a 7 day period after administering oxygen reduced blood to the hematologic malignancy patient. In an aspect, two or less transfusion-related sequelae occurs over a 7 day period after administering oxygen reduced blood to the hematologic malignancy patient. In an aspect, three transfusion-related sequelae occurs over a 7 day period after administering oxygen reduced blood to the hematologic malignancy patient. In an aspect, three or less transfusion-related sequelae occurs over a 7 day period after administering oxygen reduced blood to the hematologic malignancy patient. In an aspect, no transfusion-related sequelae occur for 14 days after administering oxygen reduced blood to the hematologic malignancy patient. In an aspect, one transfusion-related sequela occurs over a 14 day period after administering oxygen reduced blood to the hematologic malignancy patient. In an aspect, two transfusion-related sequelae occurs over a 14 day period after administering oxygen reduced blood to the hematologic malignancy patient. In an aspect, two or less transfusion-related sequelae occurs over a 14 day period after administering oxygen reduced blood to the hematologic malignancy patient. In an aspect, three transfusion-related sequelae occurs over a 14 day period after administering oxygen reduced blood to the hematologic malignancy patient. In an aspect, three or less transfusion-related sequelae occurs over a 14 day period after administering oxygen reduced blood to the hematologic malignancy patient. In an aspect, no transfusion-related sequelae occur for 21 days after administering oxygen reduced blood to the hematologic malignancy patient. In an aspect, one transfusion-related sequela occurs over a 21 day period after administering oxygen reduced blood to the hematologic malignancy patient. In an aspect, two transfusion-related sequelae occurs over a 21 day period after administering oxygen reduced blood to the hematologic malignancy patient. In an aspect, two or less transfusion-related sequelae occurs over a 21 day period after administering oxygen reduced blood to the hematologic malignancy patient. In an aspect, three transfusion-related sequelae occurs over a 21 day period after administering oxygen reduced blood to the hematologic malignancy patient. In an aspect, three or less transfusion-related sequelae occurs over a 21 day period after administering oxygen reduced blood to the hematologic malignancy patient. In an aspect, no transfusion-related sequelae occur for 28 days after administering oxygen reduced blood to the hematologic malignancy patient. In an aspect, one transfusion-related sequela occurs over a 28 day period after administering oxygen reduced blood to the hematologic malignancy patient. In an aspect, two transfusion-related sequelae occurs over a 28 day period after administering oxygen reduced blood to the hematologic malignancy patient. In an aspect, two or less transfusion-related sequelae occurs over a 28 day period after administering oxygen reduced blood to the hematologic malignancy patient. In an aspect, three transfusion-related sequelae occurs over a 28 day period after administering oxygen reduced blood to the hematologic malignancy patient. In an aspect, three or less transfusion-related sequelae occurs over a 28 day period after administering oxygen reduced blood to the hematologic malignancy patient.

In an aspect of the present disclosure, transfusion-related sequelae are selected from the group consisting of dyspnea, fever, chills, facial flushing, severe pain, shock, rapid pulse, low blood pressure, hypothermia, respiratory distress, nausea, vomiting, itching, and jaundice. In an aspect of the present disclosure, transfusion-related sequelae include dyspnea, fever, chills, facial flushing, severe pain, shock, rapid pulse, low blood pressure, hypothermia, respiratory distress, nausea, vomiting, itching, jaundice, or any combination thereof. In an aspect, the transfusion-related sequelae comprise dyspnea. In an aspect, the transfusion-related sequelae comprises fever. In an aspect, the transfusion-related sequelae comprise dyspnea. In an aspect, the transfusion-related sequelae comprises chills. In an aspect, the transfusion-related sequelae comprise dyspnea. In an aspect, the transfusion-related sequelae comprise facial flushing. In an aspect, the transfusion-related sequelae comprise severe pain. In an aspect, the transfusion-related sequelae comprises facial flushing. In an aspect, the transfusion-related sequelae comprise shock. In an aspect, the transfusion-related sequelae comprises facial flushing. In an aspect, the transfusion-related sequelae comprise rapid pulse. In an aspect, the transfusion-related sequelae comprises facial flushing. In an aspect, the transfusion-related sequelae comprise low blood pressure. In an aspect, the transfusion-related sequelae comprise hypothermia. In an aspect, the transfusion-related sequelae comprise respiratory distress. In an aspect, the transfusion-related sequelae comprise nausea. In an aspect, the transfusion-related sequelae comprises vomiting. In an aspect, the transfusion-related sequelae comprise itching. In an aspect, the transfusion-related sequelae comprise vomiting. In an aspect, the transfusion-related sequelae comprise jaundice. In an aspect, transfusion-related sequelae comprise a combination of 1, 2, 3, 4, 5, 6, 7, 8, 9, 10, 11, 12, 13, or 14 sequela.

Methods of the present disclosure provide for, and include, improving the quality of life of a hematologic malignancy patient in need thereof by administering at least two units of oxygen reduced blood, as provided herein, to the hematologic malignancy patient. In an aspect, improved quality of life is determined by evaluation of a patient's health-related quality of life (HRQOL) assessment following administration of oxygen reduced blood. HRQOL comprises a series of questions designed to assess a patient's perceived physical and mental health. In an aspect, the quality of life of the hematologic malignancy patient is improved after being administered oxygen reduced blood as assessed by the HRQOL data of the patient. In an aspect, the improved quality of life of the hematologic malignancy patient in need thereof is reduced fatigue, reduced appetite loss, reduced insomnia, reduced stress, reduced anxiety, or any combination thereof. In an aspect, the improved quality of life of the hematologic malignancy patient in need thereof is reduced fatigue. In an aspect, the improved quality of life of the hematologic malignancy patient in need thereof is reduced appetite loss. In an aspect, the improved quality of life of the hematologic malignancy patient in need thereof is reduced insomnia In an aspect, the improved quality of life of the hematologic malignancy patient in need thereof is reduced stress. In an aspect, the improved quality of life of the hematologic malignancy patient in need thereof is reduced anxiety As used herein, the singular forms “a,” “an,” and “the” include plural references unless the context clearly dictates otherwise. For example, the term “a compound” or “at least one compound” can include a plurality of compounds, including mixtures thereof.

As used herein the term “about” refers to ±10%.

As used herein the “Hemanext ONE” system is a blood container set used to process and store CPD/PAGGSM, Leukocytes reduced, and oxygen and carbon dioxide reduced red blood cells. The Hemanext ONE system is an assembly of two disposables: the Oxygen Reduction Bag (ORB) and the Hemanext (Anaerobic) Storage Bag (HSB), as outlined in International Publication Nos. WO 2016/145210 A1 and WO 2016/172645 A1 (incorporated herein by reference).

As used herein, the term “adverse event” is any unfavorable event resulting from a blood transfusion.

As used herein, the term “blood” refers to whole blood, leukoreduced red blood cells (RBCs), platelet reduced RBCs, and leukocyte and platelet reduced RBCs. The term “blood” further includes packed red blood cells, platelet reduced packed red blood cells, leukocyte reduced packed red blood cells, and leukocyte and platelet reduced packed red blood cells. The temperature of blood can vary depending on the stage of the collection process, starting at the normal body temperature of 37° C. at the time and point of collection, but decreasing rapidly to about 30° C. as soon as the blood leaves the patient's body and further thereafter to room temperature in about 6 hours when untreated, and ultimately being refrigerated at between about 4° C. and 6° C. Human red blood cells in vivo are in a dynamic state. The red blood cells contain hemoglobin, the iron-containing protein that carries oxygen throughout the body and gives red blood its color. The percentage of blood volume composed of red blood cells is called the hematocrit. As used herein, unless otherwise limited, RBCs also includes packed red blood cells (pRBCs). Packed red blood cells are prepared from whole blood using centrifugation techniques commonly known in the art. As used herein, unless otherwise indicated, the hematocrit of pRBCs is about 70%. As used herein, oxygen reduced stored RBCs can include oxygen and carbon dioxide reduced stored RBCs. As used herein, oxygen reduced (OR) blood can include oxygen and carbon dioxide (OCR) reduced blood.

As used herein, the term “hypoxic blood” refers to oxygen reduced blood or oxygen and carbon dioxide reduced blood having percent SO₂ and partial pressure of CO₂ as provided herein and in paragraph [0017].

As used herein, the terms “comprises,” “comprising,” “includes,” “including,” “having,” and their conjugates mean “including but not limited to.”

As used herein, the term “consisting of” means “including and limited to.”

As used herein, the term “consisting essentially of” means that the composition, method or structure can include additional ingredients, steps and/or parts, but only if the additional ingredients, steps and/or parts do not materially alter the basic and novel characteristics of the claimed composition, method or structure.

As used herein, the terms “higher”, “greater” or “increased” means that the measured values of oxygen reduced blood, when compared to the measured values of otherwise equivalently treated non-oxygen reduced conventionally stored blood, are at least 1 standard deviation greater, with a sample size of at least 2 for each compared measured condition.

As used herein, the terms “injury”, “damage”, and “failure” refer to an organ not functioning properly or not functioning as is expected in a person or animal without disease or injury.

As used herein, the term “less than” refers to a smaller amount and an amount greater than zero.

As used herein, the terms “patient” and “subject” are used interchangeably to mean a human or animal in need of treatment with the methods disclosed herein.

As used herein, the terms “reduce”, “reduced”, “lower”, “decreased” or “less” means that the measured values of oxygen reduced blood when compared to the measured values of otherwise equivalently treated non-oxygen reduced conventionally stored blood, are at least 1 standard deviation lower, with a sample size of at least 2 for each compared measured condition.

Various aspects of this disclosure may be presented as a fold change (e.g., a fold increase or a fold reduction). Within the context of measured values of oxygen reduced blood when compared to the measured values of otherwise equivalently treated non-oxygen reduced conventionally stored blood in the disclosure, the word “fold” should be understood to entail a multiplication in the case of a fold increase, or a division in the case of a fold reduction. For example, “increased by 2 fold” means a multiplication by 2. As another example, “reduced by 2 fold” means a division by 2.

As used herein, a “unit” of blood is about 450-500 mL including anticoagulant. Suitable anticoagulants include CPD, CPDA1, ACD, and ACD-A.

As used herein, the terms “administer”, “administration”, and “administering” refer to the delivery of blood to a patient, e.g., by transfusion.

Throughout this application, various aspects of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. Description of ranges may include terms such as “from” and “between.” For example, description of a range such as “from 1 to 6” should be considered to have specifically disclosed subranges such as “from 1 to 3,” “from 1 to 4,” “from 1 to 5,” “from 2 to 4,” “from 2 to 6,” “from 3 to 6,” etc., as well as individual numbers within that range, for example, 1, 2, 3, 4, 5, and 6. This applies regardless of the breadth of the range. As another example, description of a range such as “between 1 and 6” should be considered to have specifically disclosed subranges such as “between 1 and 3,” “between 1 and 4,” “between 1 and 5,” “between 2 and 4,” “between 2 and 6,” “between 3 and 6,” etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number “and” a second indicated number, and “ranging/ranges from” a first indicated number “to” a second indicated number are used interchangeably herein and are meant to include the first and second indicated numbers and all the fractional and integral numerals there between. Similarly, the phrases “between” a first indicated number “and” a second indicated number, and “between” a first indicated number “to” a second indicated number are also used interchangeably herein and are meant to include the first and second indicated numbers and all the fractional and integral numerals there between.

As used herein, the term “method” refers to manners, means, techniques, and procedures for accomplishing a given task including, but not limited to, administering to a human hematologic malignancy patient oxygen reduced stored blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period.

The present disclosure provides for and includes the following embodiments:

Embodiment 1. A method for reducing the blood transfusion volume for a hematologic malignancy patient in need thereof comprising administering at least two units of oxygen and carbon dioxide reduced blood to the hematologic malignancy patient in need thereof, the oxygen and carbon dioxide reduced, blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, blood are administered to the hematologic malignancy patient in need thereof in a single transfusion event, and wherein the average volume of subsequent blood transfusions administered to the hematologic malignancy patient in need thereof after the administering of the at least two units of oxygen and carbon dioxide reduced, blood is reduced by at least 10%.

Embodiment 2. The method of Embodiment 1, wherein the average volume of subsequent blood transfusions administered to the hematologic malignancy patient in need thereof after the administering of the oxygen and carbon dioxide reduced, blood is reduced by at least 25%.

Embodiment 3. The method of Embodiment 1 or Embodiment 2, wherein the average interval between the subsequent blood transfusions administered to the hematologic malignancy patient in need thereof after the administering of the oxygen and carbon dioxide reduced, blood is reduced by at least 10%.

Embodiment 4. The method of Embodiment 3, wherein the average interval between the subsequent blood transfusions administered to the hematologic malignancy patient in need thereof after the administering of the oxygen and carbon dioxide reduced, blood is reduced by at least 25%.

Embodiment 5. The method of any one of Embodiments 1 to 4, wherein a transfusion-related adverse does not occur for at least 7 days after the administering of the at least two units of oxygen and carbon dioxide reduced, blood.

Embodiment 6. The method of Embodiment 5, wherein the transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), hematoma, arterial puncture, delayed bleeding, localized infection or inflammation, brachial artery pseudoaneurysm, deep vein thrombosis (DVT), vasovagal reactions, nerve injury or irritation, compartment syndrome, arteriovenous fistula, and any combination thereof.

Embodiment 7. The method of Embodiment 5, wherein the transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death.

Embodiment 8. The method of any one of Embodiments 5 to 7, wherein the transfusion-related adverse does not occur for at least 28 days after the administering.

Embodiment 9. The method of any one of Embodiments 1 to 8, wherein the hematologic malignancy is selected from the group consisting of myelodysplastic syndromes (MDS), myelomatosis, and leukemia.

Embodiment 10. The method of Embodiment 9, wherein the myelomatosis is multiple myeloma.

Embodiment 11. The method of Embodiment 9, wherein the leukemia is acute lymphoblastic leukemia (ALL), chronic lymphocytic leukemia (CLL), acute myelogenous leukemia (AML), or chronic myelogenous leukemia (CML).

Embodiment 12. The method of any one of Embodiments 1 to 11, wherein the hematologic malignancy patient in need thereof has anemia.

Embodiment 13. The method of any one of Embodiments 1 to 12, wherein the hematologic malignancy patient in need thereof requires chronic blood transfusions.

Embodiment 14. The method of any one of Embodiments 1 to 13, wherein the hematologic malignancy patient in need thereof has a hemoglobin level of less than 9 grams per deciliter (g/dL) prior to the administering.

Embodiment 15. The method of any one of Embodiments 1 to 14, wherein the hematologic malignancy patient in need thereof has a hemoglobin level of 9 grams per deciliter (g/dL) or more after the administering.

Embodiment 16. The method of any one of Embodiments 1 to 15, wherein the hemoglobin level of the hematologic malignancy patient in need thereof is increased by at least 1.0 grams per deciliter (g/dL) after the administering.

Embodiment 17. The method of any one of Embodiments 1 to 16, wherein the blood transfusion-dependent patient has a total white blood cell concentration of less than 4.0×10⁹/L prior to the administering.

Embodiment 18. The method of Embodiment 17, wherein the total white blood cell concentration is increased to 4.0×10⁹/L or more after the administering.

Embodiment 19. The method of any one of Embodiments 1 to 18, wherein the blood transfusion-dependent patient has a neutrophil concentration of less than 1.8×10⁹/L prior to the administering.

Embodiment 20. The method of Embodiment 19, wherein the neutrophil concentration is increased to 1.8×10⁹/L or more after the administering.

Embodiment 21. The method of any one of Embodiments 1 to 20, wherein the hematologic malignancy patient in need thereof has a thrombocyte concentration of less than 145×10⁹/L prior to the administering.

Embodiment 22. The method of Embodiment 21, wherein the thrombocyte concentration is increased after the administering.

Embodiment 23. The method of any one of Embodiments 1 to 22, wherein the hematologic malignancy patient in need thereof has a reticulocyte concentration of less than 0.030×10¹²/L prior to the administering.

Embodiment 24. The method of Embodiment 23, wherein the reticulocyte concentration is increased after the administering.

Embodiment 25. The method of any one of Embodiments 1 to 24, wherein the blood of the hematologic malignancy patient in need thereof patient has a mean corpuscular volume (MCV) of greater than 98 femtoliters (fL) prior to the administering.

Embodiment 26. The method of Embodiment 25, wherein the MCV is reduced to 98 fL or less after the administering.

Embodiment 27. The method of any one of Embodiments 1 to 26, wherein the administering is over a period of about two hours.

Embodiment 28. The method of any one of Embodiments 1 to 27, wherein the storage period is less than 2 days, less than 7 days, less than 14 days, less than 21 days, less than 28 days, less than 35 days, less than 42 days, or 42 days.

Embodiment 29. The method of any one of Embodiments 1 to 28, wherein the at least two units of oxygen and carbon dioxide reduced, blood further comprise a mixture of citrate-phosphate-dextrose (CPD) and phosphate-adenosine-guanosine-glucose-saline-mannitol (PAGG-SM).

Embodiment 30. The method of any one of Embodiments 1 to 29, wherein the SO₂ is 10% or less.

Embodiment 31. The method of any one of Embodiments 1 to 30, wherein the pCO₂ is 15 mmHg or less.

Embodiment 32. A method for increasing the blood transfusion interval for a hematologic malignancy patient in need thereof comprising administering at least two units of oxygen and carbon dioxide reduced, blood to the hematologic malignancy patient in need thereof, the oxygen and carbon dioxide reduced, blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, blood are administered to the hematologic malignancy patient in need thereof in a single transfusion event, and wherein the average interval between subsequent blood transfusions administered to the hematologic malignancy patient in need thereof after the administering of the at least two units of oxygen and carbon dioxide reduced, blood is increased by at least 10%.

Embodiment 33. The method of Embodiment 32, wherein the average interval between the subsequent blood transfusions administered to the hematologic malignancy patient in need thereof after the administering of the at least two units of oxygen and carbon dioxide reduced, blood is increased by at least 25%.

Embodiment 34. The method of Embodiment 32 or Embodiment 33, wherein the average volume of the subsequent blood transfusions administered to the hematologic malignancy patient in need thereof after the administering of the at least two units of oxygen and carbon dioxide reduced, blood is reduced by at least 10%.

Embodiment 35. The method of Embodiment 34, wherein the average volume of the subsequent blood transfusions administered to the hematologic malignancy patient in need thereof after the administering of the at least two units of oxygen and carbon dioxide reduced, blood is reduced by at least 25%.

Embodiment 36. The method of any one of Embodiments 32 to 35, wherein a transfusion-related adverse event does not occur for at least 7 days after the administering of the at least two units of oxygen and carbon dioxide reduced blood.

Embodiment 37. The method of Embodiment 36, wherein the transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), hematoma, arterial puncture, delayed bleeding, localized infection or inflammation, brachial artery pseudoaneurysm, deep vein thrombosis (DVT), vasovagal reactions, nerve injury or irritation, compartment syndrome, arteriovenous fistula, and any combination thereof.

Embodiment 38. The method of Embodiment 36, wherein the transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death.

Embodiment 39. The method of any one of Embodiments 36 to 38, wherein the transfusion-related adverse event does not occur for at least 28 days after the administering.

Embodiment 40. The method of any one of Embodiments 32 to 39, wherein the hematologic malignancy is selected from the group consisting of myelodysplastic syndromes (MDS), myelomatosis, and leukemia.

Embodiment 41. The method of Embodiment 40, wherein the myelomatosis is multiple myeloma.

Embodiment 42. The method of Embodiment 40, wherein the leukemia is acute lymphoblastic leukemia (ALL), chronic lymphocytic leukemia (CLL), acute myelogenous leukemia (AML), or chronic myelogenous leukemia (CML).

Embodiment 43. The method of any one of Embodiments 32 to 42, wherein the hematologic malignancy patient in need thereof has anemia.

Embodiment 44. The method of any one of Embodiments 32 to 43, wherein the hematologic malignancy patient in need thereof requires chronic blood transfusions.

Embodiment 45. The method of any one of Embodiments 32 to 44, wherein the hematologic malignancy patient in need thereof has a hemoglobin level of less than 9 grams per deciliter (g/dL) prior to the administering.

Embodiment 46. The method of any one of Embodiments 32 to 45, wherein the hematologic malignancy patient in need thereof has a hemoglobin level of 9 grams per deciliter (g/dL) or more after the administering.

Embodiment 47. The method of any one of Embodiments 32 to 46, wherein the hemoglobin level of the hematologic malignancy patient in need thereof is increased by at least 1.0 grams per deciliter (g/dL) after the administering.

Embodiment 48. The method of any one of Embodiments 32 to 47, wherein the blood transfusion-dependent patient has a total white blood cell concentration of less than 4.0×10⁹/L prior to the administering.

Embodiment 49. The method of Embodiment 48, wherein the total white blood cell concentration is increased to 4.0×10⁹/L or more after the administering.

Embodiment 50. The method of any one of Embodiments 32 to 49, wherein the blood transfusion-dependent patient has a neutrophil concentration of less than 1.8×10⁹/L prior to the administering.

Embodiment 51. The method of Embodiment 50, wherein the neutrophil concentration is increased to 1.8×10⁹/L or more after the administering.

Embodiment 52. The method of any one of Embodiments 32 to 51, wherein the hematologic malignancy patient in need thereof has a thrombocyte concentration of less than 145×10⁹/L prior to the administering.

Embodiment 53. The method of Embodiment 52, wherein the thrombocyte concentration is increased after the administering.

Embodiment 54. The method of any one of Embodiments 32 to 53, wherein the hematologic malignancy patient in need thereof has a reticulocyte concentration of less than 0.030×10¹²/L prior to the administering.

Embodiment 55. The method of Embodiment 54, wherein the reticulocyte concentration is increased after the administering.

Embodiment 56. The method of any one of Embodiments 32 to 55, wherein the blood of the hematologic malignancy patient in need thereof patient has a mean corpuscular volume (MCV) of greater than 98 femtoliters (fL) prior to the administering.

Embodiment 57. The method of Embodiment 56, wherein the MCV is reduced to 98 fL or less after the administering.

Embodiment 58. The method of any one of Embodiments 32 to 57, wherein the administering is over a period of about two hours.

Embodiment 59. The method of any one of Embodiments 32 to 58, wherein the storage period is less than 2 days, less than 7 days, less than 14 days, less than 21 days, less than 28 days, less than 35 days, less than 42 days, or 42 days.

Embodiment 60. The method of any one of Embodiments 32 to 59, wherein the at least two units of oxygen and carbon dioxide reduced, blood further comprise a mixture of citrate-phosphate-dextrose (CPD) and phosphate-adenosine-guanosine-glucose-saline-mannitol (PAGG-SM).

Embodiment 61. The method of any one of Embodiments 32 to 60, wherein the SO₂ is 10% or less.

Embodiment 62. The method of any one of Embodiments 32 to 61, wherein the pCO₂ is 15 mmHg or less.

Embodiment 63. A method for increasing the hemoglobin increment of a hematologic malignancy patient in need thereof comprising administering at least two units of oxygen and carbon dioxide reduced blood to the hematologic malignancy patient in need thereof, the oxygen and carbon dioxide reduced, blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, blood are administered to the hematologic malignancy patient in need thereof in a single transfusion event, and wherein the hemoglobin increment of the hematologic malignancy patient in need thereof is increased by at least 1.0 grams per deciliter (g/dL) after the administering.

Embodiment 64. The method of Embodiment 63, wherein iron overload is reduced in the hematologic malignancy patient in need thereof after the administering.

Embodiment 65. The method of Embodiment 63 or Embodiment 64, wherein the hematologic malignancy patient in need thereof has a hemoglobin level of less than 9 grams per deciliter (g/dL) prior to the administering.

Embodiment 66. The method of any one of Embodiments 63 to 65, wherein the hematologic malignancy patient in need thereof has a hemoglobin level of 9 grams per deciliter (g/dL) or more after the administering.

Embodiment 67. The method of any one of Embodiments 63 to 66, wherein a transfusion-related adverse does not occur for at least 7 days after the administering of the at least two units of oxygen and carbon dioxide reduced, blood.

Embodiment 68. The method of Embodiment 67, wherein the transfusion-related adverse event is selected from the group consisting of hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), hematoma, arterial puncture, delayed bleeding, localized infection or inflammation, brachial artery pseudoaneurysm, deep vein thrombosis (DVT), vasovagal reactions, nerve injury or irritation, compartment syndrome, arteriovenous fistula, and any combination thereof.

Embodiment 69. The method of Embodiment 67, wherein the transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death.

Embodiment 70. The method of any one of Embodiments 67 to 69, wherein the transfusion-related adverse does not occur for at least 28 days after the administering.

Embodiment 71. The method of any one of Embodiments 67 to 70, wherein the hematologic malignancy is selected from the group consisting of myelodysplastic syndromes (MDS), myelomatosis, and leukemia.

Embodiment 72. The method of Embodiment 71, wherein the myelomatosis is multiple myeloma.

Embodiment 73. The method of Embodiment 71, wherein the leukemia is acute lymphoblastic leukemia (ALL), chronic lymphocytic leukemia (CLL), acute myelogenous leukemia (AML), or chronic myelogenous leukemia (CML).

Embodiment 74. The method of any one of Embodiments 63 to 73, wherein the hematologic malignancy patient in need thereof has anemia.

Embodiment 75. The method of any one of Embodiments 63 to 74, wherein the hematologic malignancy patient in need thereof requires chronic blood transfusions.

Embodiment 76. The method of any one of Embodiments 63 to 75, wherein the average volume of subsequent blood transfusions administered to the hematologic malignancy patient in need thereof after the administering of the at least two units of oxygen and carbon dioxide reduced, blood is reduced.

Embodiment 77. The method of any one of Embodiments 63 to 76, wherein the average interval between subsequent blood transfusions administered to the hematologic malignancy patient in need thereof after the administering of the at least two units of oxygen and carbon dioxide reduced, blood is increased.

Embodiment 78. The method of any one of Embodiments 63 to 77, wherein the administering is over a period of about two hours.

Embodiment 79. The method of any one of Embodiments 63 to 78, wherein the storage period is less than 2 days, less than 7 days, less than 14 days, less than 21 days, less than 28 days, less than 35 days, less than 42 days, or 42 days.

Embodiment 80. The method of any one of Embodiments 63 to 79, wherein the at least two units of oxygen and carbon dioxide reduced blood further comprise a mixture of citrate-phosphate-dextrose (CPD) and phosphate-adenosine-guanosine-glucose-saline-mannitol (PAGG-SM).

Embodiment 81. The method of any one of Embodiments 63 to 80, wherein the SO₂ is 10% or less.

Embodiment 82. The method of any one of Embodiments 63 to 81, wherein the pCO₂ is 15 mmHg or less.

Embodiment 83. A method for improving oxygen delivery to a hematologic malignancy patient in need thereof comprising administering at least two units of oxygen and carbon dioxide reduced, blood to the hematologic malignancy patient in need thereof, the oxygen and carbon dioxide reduced, blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, blood are administered to the hematologic malignancy patient in need thereof in a single transfusion event, and wherein the blood oxygen level of the hematologic malignancy patient in need thereof is increased by at least 10% after the administering.

Embodiment 84. The method of Embodiment 83, wherein the blood oxygen level of the hematologic malignancy patient in need thereof is increased by at least 25% after the administering.

Embodiment 85. The method of Embodiment 83 or Embodiment 84, wherein the blood oxygen level of the hematologic malignancy patient in need thereof is less than 90% prior to the administering.

Embodiment 86. The method of any one of Embodiments 83 to 85, wherein the blood oxygen level of the hematologic malignancy patient in need thereof is at least 95% prior to the administering.

Embodiment 87. The method of any one of Embodiments 83 to 86, wherein the hematologic malignancy patient in need thereof has a hemoglobin level of less than 9 grams per deciliter (g/dL) prior to the administering.

Embodiment 88. The method of any one of Embodiments 83 to 87, wherein the hematologic malignancy patient in need thereof has a hemoglobin level of 9 grams per deciliter (g/dL) or more after the administering.

Embodiment 89. The method of any one of Embodiments 83 to 88, wherein a transfusion-related adverse does not occur for at least 7 days after the administering of the at least two units of oxygen and carbon dioxide reduced, blood.

Embodiment 90. The method of Embodiment 89, wherein the transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), hematoma, arterial puncture, delayed bleeding, localized infection or inflammation, brachial artery pseudoaneurysm, deep vein thrombosis (DVT), vasovagal reactions, nerve injury or irritation, compartment syndrome, arteriovenous fistula, and any combination thereof.

Embodiment 91. The method of Embodiment 89, wherein the transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death.

Embodiment 92. The method of any one of Embodiments 89 to 91, wherein the transfusion-related adverse event does not occur for at least 28 days after the administering.

Embodiment 93. The method of any one of Embodiments 89 to 92, wherein the hematologic malignancy is selected from the group consisting of myelodysplastic syndromes (MDS), myelomatosis, and leukemia.

Embodiment 94. The method of Embodiment 93, wherein the myelomatosis is multiple myeloma.

Embodiment 95. The method of Embodiment 93, wherein the leukemia is acute lymphoblastic leukemia (ALL), chronic lymphocytic leukemia (CLL), acute myelogenous leukemia (AML), or chronic myelogenous leukemia (CML).

Embodiment 96. The method of any one of Embodiments 89 to 95, wherein the hematologic malignancy patient in need thereof has anemia.

Embodiment 97. The method of any one of Embodiments 89 to 96, wherein the hematologic malignancy patient in need thereof requires chronic blood transfusions.

Embodiment 98. The method of any one of Embodiments 89 to 97, wherein the average volume of subsequent blood transfusions administered to the hematologic malignancy patient in need thereof after the administering of the at least two units of oxygen and carbon dioxide reduced blood is reduced.

Embodiment 99. The method of any one of Embodiments 89 to 98, wherein the average interval between subsequent blood transfusions administered to the hematologic malignancy patient in need thereof after the administering of the at least two units of oxygen and carbon dioxide reduced blood is increased.

Embodiment 100. The method of any one of Embodiments 89 to 99, wherein the administering is over a period of about two hours.

Embodiment 101. The method of any one of Embodiments 89 to 100, wherein the storage period is less than 2 days, less than 7 days, less than 14 days, less than 21 days, less than 28 days, less than 35 days, less than 42 days, or 42 days.

Embodiment 102. The method of any one of Embodiments 89 to 101, wherein the at least two units of oxygen and carbon dioxide reduced, blood further comprise a mixture of citrate-phosphate-dextrose (CPD) and phosphate-adenosine-guanosine-glucose-saline-mannitol (PAGG-SM).

Embodiment 103. The method of any one of Embodiments 89 to 102, wherein the SO₂ is 10% or less.

Embodiment 104. The method of any one of Embodiments 89 to 103, wherein the pCO₂ is 15 mmHg or less.

Embodiment 105. A method for preventing or reducing transfusion-related sequelae in a hematologic malignancy patient in need thereof comprising administering at least two units of oxygen and carbon dioxide reduced, blood to the hematologic malignancy patient in need thereof, the oxygen and carbon dioxide reduced, blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, blood are administered to the hematologic malignancy patient in need thereof in a single transfusion event, and wherein the occurrence of transfusion-related sequelae is reduced over a 7 day period after the administering compared to a hematologic malignancy patient in need thereof administered at least two units of conventional blood.

Embodiment 106. The method of Embodiment 105, wherein less than two transfusion-related sequelae occur over the 7 day period.

Embodiment 107. The method of Embodiment 105 or Embodiment 106, wherein the occurrence of transfusion-related sequelae is reduced over a 28 day period after the administering compared to a hematologic malignancy patient in need thereof administered at least two units of conventional blood.

Embodiment 108. The method of Embodiment 107, wherein less than two transfusion-related sequelae occur over the 28 day period.

Embodiment 109. The method of any one of Embodiments 105 to 108, wherein the transfusion-related sequelae are selected from the group consisting of dyspnea, fever, chills, facial flushing, severe pain, shock, rapid pulse, low blood pressure, hypothermia, respiratory distress, nausea, vomiting, itching, and jaundice.

Embodiment 110. The method of any one of Embodiments 105 to 109, wherein the hematologic malignancy patient in need thereof has a hemoglobin level of less than 9 grams per deciliter (g/dL) prior to the administering.

Embodiment 111. The method of any one of Embodiments 105 to 110, wherein the hematologic malignancy patient in need thereof has a hemoglobin level of 9 grams per deciliter (g/dL) or more after the administering.

Embodiment 112. The method of any one of Embodiments 105 to 111, wherein the hematologic malignancy is selected from the group consisting of myelodysplastic syndromes (MDS), myelomatosis, and leukemia.

Embodiment 113. The method of Embodiment 112, wherein the myelomatosis is multiple myeloma.

Embodiment 114. The method of Embodiment 112, wherein the leukemia is acute lymphoblastic leukemia (ALL), chronic lymphocytic leukemia (CLL), acute myelogenous leukemia (AML), or chronic myelogenous leukemia (CML).

Embodiment 115. The method of any one of Embodiments 105 to 114, wherein the hematologic malignancy patient in need thereof has anemia.

Embodiment 116. The method of any one of Embodiments 105 to 115, wherein the hematologic malignancy patient in need thereof requires chronic blood transfusions.

Embodiment 117. The method of any one of Embodiments 105 to 116, wherein the average volume of subsequent blood transfusions administered to the hematologic malignancy patient in need thereof after the administering of the at least two units of oxygen and carbon dioxide reduced, blood is reduced.

Embodiment 118. The method of any one of Embodiments 105 to 117, wherein the average interval between subsequent blood transfusions administered to the hematologic malignancy patient in need thereof after the administering of the at least two units of oxygen and carbon dioxide reduced, blood is increased.

Embodiment 119. The method of any one of Embodiments 105 to 118, wherein the administering is over a period of about two hours.

Embodiment 120. The method of any one of Embodiments 105 to 119, wherein the storage period is less than 2 days, less than 7 days, less than 14 days, less than 21 days, less than 28 days, less than 35 days, less than 42 days, or 42 days.

Embodiment 121. The method of any one of Embodiments 105 to 120, wherein the at least two units of oxygen and carbon dioxide reduced, blood further comprise a mixture of citrate-phosphate-dextrose (CPD) and phosphate-adenosine-guanosine-glucose-saline-mannitol (PAGG-SM).

Embodiment 122. The method of any one of Embodiments 105 to 121, wherein the SO₂ is 10% or less.

Embodiment 123. The method of any one of Embodiments 105 to 122, wherein the pCO₂ is 15 mmHg or less.

Embodiment 124. A method for improving the quality of life of a hematologic malignancy patient in need thereof comprising administering at least two units of oxygen and carbon dioxide reduced, blood to the hematologic malignancy patient in need thereof, the oxygen and carbon dioxide reduced, blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, blood are administered to the hematologic malignancy patient in need thereof in a single transfusion event, and wherein the quality of life of the hematologic malignancy patient in need thereof is improved after the administering as assessed by the health-related quality of life (HRQOL) data of the hematologic malignancy patient in need thereof.

Embodiment 125. The method of Embodiment 124, wherein the improved quality of life of the hematologic malignancy patient in need thereof is reduced fatigue, reduced appetite loss, reduced insomnia, reduced stress, reduced anxiety, or any combination thereof.

Embodiment 126. The method of Embodiment 124 or Embodiment 125, wherein the storage period is less than 2 days, less than 7 days, less than 14 days, less than 21 days, less than 28 days, less than 35 days, less than 42 days, or 42 days.

Embodiment 127. The method of any one of Embodiments 124 to 126, wherein the at least two units of oxygen and carbon dioxide reduced, blood further comprise a mixture of citrate-phosphate-dextrose (CPD) and phosphate-adenosine-guanosine-glucose-saline-mannitol (PAGG-SM).

Embodiment 128. The method of any one of Embodiments 124 to 127, wherein the SO₂ is 10% or less.

Embodiment 129. The method of any one of Embodiments 124 to 128, wherein the pCO₂ is 15 mmHg or less.

Embodiment 130. A method for reducing the blood transfusion volume, increasing the blood transfusion interval, or both in a patient having myelodysplastic syndromes (MDS) in need thereof comprising administering between one to three units of oxygen and carbon dioxide reduced, blood to the patient having MDS in need thereof, the oxygen and carbon dioxide reduced, blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the patient having MDS in need thereof is a blood transfusion dependent patient, wherein the one to three units of oxygen and carbon dioxide reduced, blood are administered to the patient having MDS in need thereof in a single transfusion event.

Embodiment 131. The method of Embodiment 130, wherein the average volume of subsequent blood transfusions administered to the patient having MDS in need thereof after the administering of the one to three units of oxygen and carbon dioxide reduced, blood is reduced by at least 10%.

Embodiment 132. The method of Embodiment 130 or Embodiment 131, wherein the average interval between subsequent blood transfusions administered to the patient having MDS in need thereof after the administering of the one to three units of oxygen and carbon dioxide reduced blood is increased by at least 10%.

Embodiment 133. The method of any one of Embodiment 130 to 132, wherein the MDS is very low, low, or intermediate risk MDS as determined by the Revised International Prognostic Scoring System (IPSS-R).

Embodiment 134. The method of Embodiment 133, wherein a bone marrow aspirate of the patient having MDS in need thereof completed up to six months prior to the administering did not indicate progression to higher risk MDS.

Embodiment 135. The method of any one of Embodiments 130 to 134, wherein the patient having MDS in need thereof requires at least two units of conventional blood every four weeks prior to the administering.

Embodiment 136. The method of any one of Embodiments 130 to 135, wherein the patient having MDS in need thereof has an Eastern Cooperative Oncology Group (ECOG) performance status of 3 or less prior to the administering.

Embodiment 137. The method of any one of Embodiments 130 to 136, wherein the patient having MDS in need thereof has a hemoglobin level of 8 grams per deciliter (g/dL) or less prior to the administering.

Embodiment 138. The method of any one of Embodiments 130 to 137, wherein the patient having MDS in need thereof has a hemoglobin level of 9 grams per deciliter (g/dL) or more after the administering.

Embodiment 139. The method of any one of Embodiments 130 to 138, wherein the hematocrit of the patient having MDS in need thereof is increased after the administering.

Embodiment 140. The method of any one of Embodiments 130 to 139, wherein the patient having MDS in need thereof has an elevated serum ferritin level prior to the administering.

Embodiment 141. The method of Embodiment 140, wherein the elevated serum ferritin level is greater than 200 nanograms per milliliter (ng/mL) for a female patient having MDS in need thereof, and greater than 300 ng/mL for a male patient having MDS in need thereof.

Embodiment 142. The method of Embodiment 140 or Embodiment 141, wherein the serum ferritin level of the patient having MDS in need thereof is reduced after the administering.

Embodiment 143. The method of Embodiment 142, wherein the reduced serum ferritin level is 200 nanograms per milliliter (ng/mL) or less for a female patient having MDS in need thereof, or 300 ng/mL or less for a male patient having MDS in need thereof.

Embodiment 144. The method of any one of Embodiments 130 to 143, wherein a transfusion-related adverse event does not occur for at least 7 days after the administering of the one to three units of oxygen and carbon dioxide reduced blood to the patient having MDS in need thereof.

Embodiment 145. The method of Embodiment 144, wherein the transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), hematoma, arterial puncture, delayed bleeding, localized infection or inflammation, brachial artery pseudoaneurysm, deep vein thrombosis (DVT), vasovagal reactions, nerve injury or irritation, compartment syndrome, arteriovenous fistula, and any combination thereof.

Embodiment 146. The method of Embodiment 144, wherein the transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death.

Embodiment 147. The method of any one of Embodiments 144 to 146, wherein the transfusion-related adverse event does not occur for at least 28 days after the administering.

Embodiment 148. The method of any one of Embodiments 130 to 147, wherein the administering is once a month for a period of at least 6 months.

Embodiment 149. The method of Embodiment 148, wherein the Quality of Life in Myelodysplasia Scale (QUALMS) score of the patient having MDS in need thereof is improved after the period of at least 6 months.

Embodiment 150. The method of Embodiment 148, wherein the EORTC Core Quality of Life questionnaire (EORTC QLQ-C30) score of the patient having MDS in need thereof is improved after the period of at least 6 months.

Embodiment 151. The method of any one of Embodiments 148 to 150, wherein a serum ferritin level of 200 nanograms per milliliter (ng/mL) or less for a female patient having MDS in need thereof, or 300 ng/mL or less for a male patient having MDS in need thereof, is maintained throughout the period of at least six months.

Embodiment 152. The method of any one of Embodiments 130 to 151, wherein one unit of oxygen and carbon dioxide reduced, blood is administered to the blood transfusion-dependent patient having MDS.

Embodiment 153. The method of any one of Embodiments 130 to 151, wherein two units of oxygen and carbon dioxide reduced, blood are administered to the blood transfusion-dependent patient having MDS.

Embodiment 154. The method of any one of Embodiments 130 to 151, wherein three units of oxygen and carbon dioxide reduced, blood are administered to the blood transfusion-dependent patient having MDS.

Embodiment 155. The method of any one of Embodiments 130 to 154, wherein the storage period is less than 2 days, less than 7 days, less than 14 days, less than 21 days, less than 28 days, less than 35 days, less than 42 days, or 42 days.

Embodiment 156. The method of any one of Embodiments 130 to 155, wherein the oxygen and carbon dioxide reduced, blood further comprises a mixture of citrate-phosphate-dextrose (CPD) and phosphate-adenosine-guanosine-glucose-saline-mannitol (PAGG-SM).

Embodiment 157. The method of any one of Embodiments 130 to 156, wherein the SO₂ is 10% or less.

Embodiment 158. The method of any one of Embodiments 130 to 157, wherein the pCO₂ is 15 mmHg or less.

Embodiment 159. A method for preventing the occurrence of a transfusion-related adverse event hematologic malignancy patient in need thereof comprising administering at least two units of oxygen and carbon dioxide reduced blood to the hematologic malignancy patient in need thereof, the oxygen and carbon dioxide reduced, blood having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, blood are administered to the hematologic malignancy patient in need thereof in a single transfusion event, and wherein the transfusion-related adverse does not occur for at least 7 days after the administering.

Embodiment 160. The method of Embodiment 159, wherein the transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), hematoma, arterial puncture, delayed bleeding, localized infection or inflammation, brachial artery pseudoaneurysm, deep vein thrombosis (DVT), vasovagal reactions, nerve injury or irritation, compartment syndrome, arteriovenous fistula, and any combination thereof.

Embodiment 161. The method of Embodiment 159, wherein the transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death.

Embodiment 162. The method of any one of Embodiments 159 to 161, wherein the transfusion-related adverse does not occur for at least 28 days after the administering.

Embodiment 163. The method of any one of Embodiments 159 to 162, wherein the hematologic malignancy is selected from the group consisting of myelodysplastic syndromes (MDS), myelomatosis, and leukemia.

Embodiment 164. The method of Embodiment 163, wherein the myelomatosis is multiple myeloma.

Embodiment 165. The method of Embodiment 164, wherein the leukemia is acute lymphoblastic leukemia (ALL), chronic lymphocytic leukemia (CLL), acute myelogenous leukemia (AML), or chronic myelogenous leukemia (CML).

Embodiment 166. The method of any one of Embodiments 159 to 165, wherein the hematologic malignancy patient in need thereof has anemia.

Embodiment 167. The method of any one of Embodiments 159 to 166, wherein the hematologic malignancy patient in need thereof requires chronic blood transfusions.

Embodiment 168. The method of any one of Embodiments 159 to 167, wherein the hematologic malignancy patient in need thereof has a hemoglobin level of less than 9.0 g/dL prior to the administering.

Embodiment 169. The method of any one of Embodiments 159 to 168, wherein the hematologic malignancy patient in need thereof has a hemoglobin level of 9 grams per deciliter (g/dL) or more after the administering.

Embodiment 170. The method of any one of Embodiments 159 to 169, wherein the hemoglobin level of the hematologic malignancy patient in need thereof is increased by at least 1.0 grams per deciliter (g/dL) after the administering.

Embodiment 171. The method of any one of Embodiments 159 to 170, wherein the average volume of subsequent blood transfusions administered to the hematologic malignancy patient in need thereof after the administering of the at least two units of oxygen and carbon dioxide reduced, blood is reduced.

Embodiment 172. The method of any one of Embodiments 159 to 171, wherein the average interval between subsequent blood transfusions administered to the hematologic malignancy patient in need thereof after the administering of the at least two units of oxygen and carbon dioxide reduced blood is increased.

Embodiment 173. The method of any one of Embodiments 159 to 172, wherein the administering is over a period of about two hours.

Embodiment 174. The method of any one of Embodiments 159 to 173, wherein the storage period is less than 2 days, less than 7 days, less than 14 days, less than 21 days, less than 28 days, less than 35 days, less than 42 days, or 42 days.

Embodiment 175. The method of any one of Embodiments 159 to 174, wherein the at least two units of oxygen and carbon dioxide reduced, blood further comprise a mixture of citrate-phosphate-dextrose (CPD) and phosphate-adenosine-guanosine-glucose-saline-mannitol (PAGG-SM).

Embodiment 176. The method of any one of Embodiments 159 to 175, wherein the SO₂ is 10% or less.

Embodiment 177. The method of any one of Embodiments 159 to 176, wherein the pCO₂ is 15 mmHg or less.

Embodiment 178. Use of donor blood to manufacture oxygen and carbon dioxide reduced, blood having an oxygen saturation of (SO₂) of 20% or less and an initial partial pressure of carbon dioxide (pCO₂) of 30 millimeters of mercury (mmHg) or less prior to and during storage for the therapeutic application of reducing the blood transfusion volume for a hematologic malignancy patient in need thereof.

Embodiment 179. Use of donor blood to manufacture oxygen and carbon dioxide reduced, blood having an oxygen saturation of (SO₂) of 20% or less and an initial partial pressure of carbon dioxide (pCO₂) of 30 millimeters of mercury (mmHg) or less prior to and during storage for the therapeutic application of increasing the blood transfusion interval for a hematologic malignancy patient in need thereof.

Embodiment 180. Use of donor blood to manufacture oxygen and carbon dioxide reduced, blood having an oxygen saturation of (SO₂) of 20% or less and an initial partial pressure of carbon dioxide (pCO₂) of 30 millimeters of mercury (mmHg) or less prior to and during storage for the therapeutic application of reducing the blood transfusion volume, increasing the blood transfusion interval, or both in a hematologic malignancy patient in need thereof.

Embodiment 181. Use of donor blood to manufacture oxygen and carbon dioxide reduced, blood having an oxygen saturation of (SO₂) of 20% or less and an initial partial pressure of carbon dioxide (pCO₂) of 30 millimeters of mercury (mmHg) or less prior to and during storage for the therapeutic application of reducing the blood transfusion volume, increasing the blood transfusion interval, or both in a patient having myelodysplastic syndromes (MDS) in need thereof.

Embodiment 182. Use of donor blood to manufacture oxygen and carbon dioxide reduced, blood having an oxygen saturation of (SO₂) of 20% or less and an initial partial pressure of carbon dioxide (pCO₂) of 30 millimeters of mercury (mmHg) or less prior to and during storage for the therapeutic application of preventing or reducing the risk of a transfusion-related adverse event in a hematologic malignancy patient in need thereof.

Embodiment 183. The use of Embodiment 182, wherein the transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), hematoma, arterial puncture, delayed bleeding, localized infection or inflammation, brachial artery pseudoaneurysm, deep vein thrombosis (DVT), vasovagal reactions, nerve injury or irritation, compartment syndrome, arteriovenous fistula, and any combination thereof.

Embodiment 184. The use of Embodiment 182, wherein the transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death.

Embodiment 185. The method of any one of Embodiments 1, 32, 63, 83, 105, 124, 130, and 159, wherein the blood is selected from the group consisting of whole blood, leukoreduced red blood cells (RBCs), platelet reduced RBCs, leukocyte and platelet reduced RBCs, packed red blood cells, platelet reduced packed red blood cells, leukocyte reduced packed red blood cells, and leukocyte and platelet reduced packed red blood cells.

Embodiment 186. The method of any one of Embodiments 1, 32, 63, 83, 105, 124, 130, and 159, wherein said blood comprises red blood cells.

Embodiment 187. The method of any one of Embodiments 1, 32, 63, 83, 105, 124, 130, and 159, wherein said blood comprises leukoreduced red blood cells.

### EXAMPLES

### Example 1: Testing of Red Blood Cell Infusion

Fourteen (14) participants receive two separate infusions of approximately 10 mL of autologous radiolabeled red blood cells (RBCs). One of the infusions is with hypoxic RBCs in AS-3 produced using the Hemanext ONE process as outlined in International Publication Nos. WO 2016/145210 A1 and WO 2016/172645 A1 (incorporated herein by reference) and the other transfusion is with conventional RBCs in AS-3. The hypoxic RBCs infused have an overall higher dual-label 24 hour in vivo recovery (mean: 89.3, SD: 5.81) than the conventional RBCs (mean: 85.8, SD: 6.12). None of the participants experience any adverse events (AE) or serious adverse events (SAE) related to the use of hypoxic RBCs.

### Example 2: Treatment of Hematological Malignancy Patients

Ten patents with hematological malignancies, who require transfusion of red blood cells and who fulfill eligibility criteria are enrolled in a study to test the safety of oxygen and carbon dioxide reduced and leukoreduced red blood cells for transfusion. Patients meeting the following criteria are included in the clinical investigation and undergo the informed consent process:
1. Male or female patients at least 18 years of age.
2. Patients expected to require >2 units of red blood cells in a single transfusion event.
3. Patients who have the capacity to consent to participate and are willing to comply with the study procedures.
4. Patients identified by a transfusion hemoglobin trigger of less than 9 g/dL.
5. Patients with a documented diagnosis of leukemia, myelomatosis or MDS requiring chronic transfusions.

Patients meeting any of the following exclusion criteria are excluded from participation in the clinical investigation:
1. Patients with any positive antibody screening test.
2. Patients for whom consent has not been obtained.
3. Patients with a known hemolytic anemia (congenital or acquired).
4. Patients <18 years old.
5. Patients with a known or suspected pregnancy.
6. Patients with a history of major transfusion reactions.
7. Patients whom the Investigator deems clinical trial participation is not in their best interest.

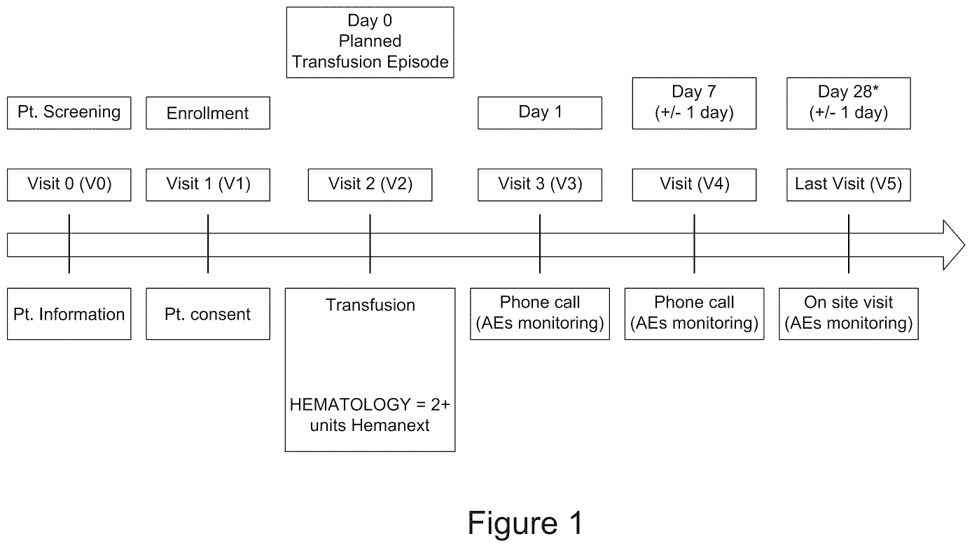

Patients are transfused with 2 units of hypoxic blood (i.e., oxygen reduced blood, or oxygen and carbon dioxide reduced blood) during scheduled transfusion therapy visits. The remainder of the units required for transfusion are drawn from traditionally processed red cells. Adverse events are collected from the Screening visit through either discharge, the subsequent transfusion, or Day 28, whichever comes first. See FIG. 1. Patients are evaluated over five visits. The primary objective of the study is to determine the safety and tolerance of hypoxic RBCs up to 24 hours following the transfusion initiation and overall up to 7 days (+/−1 day) after the transfusion episode (single transfusion course). The secondary objectives are as follows:
1. Assessment of pre- and post-transfusion hemoglobin levels;
2. Assessment of hemoglobin level before the subsequent transfusion, if applicable;
3. Assessment of the occurrence of adverse events (AEs):
   a. Up to 7 days (+/−1 day) post transfusion, in comparison with historical control published in local registries (including but not limited to infection, deep vein thrombosis, acute respiratory distress syndrome, transfusion-related acute lung injury, transfusion associated circulatory overload, anaphylactic shock, acute hemolytic transfusion reaction).
   b. Up to the subsequent transfusion episode or up to 28 days after the initial transfusion, whichever comes first.
   c. From enrollment, up to their subsequent transfusion or 28 days post-transfusion, whichever comes first, through the assessment of patient's diary;
4. Assessment of the vital signs during and up to 15 minutes after the transfusion.

The primary outcome is to determine the number of participants who experience an adverse event (all types/grades) within a time frame. Patients are monitored for adverse events throughout the entirety of their participation in the clinical investigation. Investigators monitor patients directly for adverse events during their transfusion visit. In addition, patients are contacted by phone 24 hours and 7 days (+/−1) after a transfusion visit to record any adverse events experienced since their transfusion visit. All adverse events are recorded.

Patients are monitored for adverse events throughout the entirety of their participation in the clinical investigation. Adverse events are elicited from the patients, the medical record, and an adverse event diary. Enrolled patients undergo standard care for their disease or illness as prescribed by the Investigators. All procedures are performed in compliance with the site's established policies and work instructions. The type and the grade of each adverse event are categorized according to:
1. Association for the Advancement of Blood and Biotherapies (AABB) technical manual, 20^{th} edition (2020);
2. Delaney et al., “Biomedical Excellence for Safer Transfusion (BEST) Collaborative. Transfusion reactions: prevention, diagnosis, and treatment,” Lancet 388:2825-2836 (2016);
3. Local AEs database;
4. ISO 14155-2020 (Clinical investigation of medical devices for human subjects—Good Clinical Practice) definitions.

Patients are also monitored for secondary outcomes related to AE occurrence during their transfusion visit and then patients are contacted by phone 24 hours and 7 days (+/−1) after their transfusion visit to record any adverse events they experienced since their transfusion visit. The patient's chart is checked for any AE occurrence after their following transfusion visit or 28 days after their initial transfusion, whichever comes first. All adverse events are recorded.

The secondary outcomes measured include, but are not limited to, the following:
1. Evolution of the hemoglobin level before and after the transfusion;
2. Calculation of the hemoglobin increment after transfusion corrected for patient blood volume and hemoglobin dose;
3. Comparison of the hemoglobin level before the index transfusion to that prior to the subsequent transfusion;
4. Comparison of the frequency of all AEs, including transfusion related AEs up to 7 days (+/−1) post-transfusion compared to historical data in local patient registries;
5. Evaluation of AEs from enrollment, up to prior to the subsequent transfusion or up to 28 days after the initial transfusion, whichever comes first;
6. Evaluation of the frequency of all AEs, from enrollment, up to their subsequent transfusion or 28 days post-transfusion, whichever comes first, through the assessment of patient's diary, when applicable;
7. Evaluation of patient's vital signs over the course of the transfusion and up to 15 minutes post-transfusion.

Patient information is collected from enrollment and throughout the end of the study visit as outlined in Table 1.

TABLE 1
Hematological Malignancies Screening, Transfusion, and Follow-up Guidelines

| | Investigation Periods | | | | | | |
|---|---|---|---|---|---|---|---|
| | Screening & | | | | | | |
| | Enrollment | Pre- | | Post- | Follow | Follow- | Follow- |
| | Visit | Transfusion | Transfusion | Transfusion | Up 1 | up 2 | up 3 |
| | Visit | | | | | | |
| | 1 | 2 | 2 | 2 | 3 | 4 | 5 |
| | Time (hour, day, week) | | | | | | |
| | | | | | | | Day of |
| | | | | | | | subsequent |
| | | | | | | | transfusion |
| | | | | | | | or D 28, |
| | | | | | D 2 | D 7 (+/−1 | whichever |
| | D 0 | D 1 | D 1 | D 1 | (≤24 h) | day) | comes 1st |
| In-/Exclusion | x | x | | | | | |
| Patient | | | | | | | |
| Information | | | | | | | |
| Patient consent | x | | | | | | |
| (ICF) | | | | | | | |
| Demographics | x | | | | | | |
| Medical History | x | | | | | | |
| Concomitant | x | x | x | x | x | x | x |
| Medications | | | | | | | |
| Physical | x | | | | | | x |
| Examination | | | | | | | |
| Pregnancy Test | x | | | | | | |
| Height | x | | | | | | |
| Weight | x | | | | | | |
| RBC Unit Weight | | x | | | | | |
| Patient | | x | | x | | | x |
| Hemoglobin | | | | | | | |
| Blood Pressure² | x | x | x | x | | | |
| Heart Rate² | x | x | x | x | | | |
| Body | x | x | x | x | | | |
| Temperature² | | | | | | | |
| Respiratory Rate² | x | x | x | x | | | |
| Oxygen | x | x | x | x | | | |
| Saturation¹ | | | | | | | |
| Transfusion | | | x | | | | |
| Adverse Events, | x | x | x | x | x | x | x |
| Adverse device | | | | | | | |
| effects | | | | | | | |
| Review Device | | x | x | x | | | |
| (blood bag) | | | | | | | |
| Integrity | | | | | | | |
| Patient Diary | x | x | x | x | x | x | x |

¹Oxygen saturation is measured by non-invasive pulse oximetry during the transfusion and for 15 minutes post-transfusion.
²Measured during the transfusion and for 15 minutes post-transfusion.

During the first follow-up visit 24 hours post-transfusion infection patients are evaluated for infection occurrence, adverse event occurrence, concomitant medications, and device deficiencies. The second follow-up visit occurs over the phone 7 days (+/−1) post-transfusion. Patients are evaluated for the occurrence of infection, adverse event(s), concomitant medications, device deficiencies, and patient diary. The final follow-up occurs at the patient's subsequent transfusion visit or 28 days after transfusion, whichever comes first. Patients are evaluated for the occurrence of adverse event(s) and concomitant medications, pre-transfusion hemoglobin, and a physical examination is performed.

### Example 3: Hypoxic Red Blood Cells Transfused in a Myelodysplastic Syndromes (MDS) Patient

An 80-year-old male patient is enrolled in the transfusion study. He initially presented with anaemia and diagnostic workup revealed bone marrow smear with morphological features of erythroid dysplasia with ring sideroblasts in 30% of cells and mutation in the SF3B1 gene. Prior to study enrollment, the patient became transfusion-dependent after an initial transfusion with conventional RBCs, and received 2 units of RBCs every 2 weeks for the last year. The patient is transfused with 2 units of hypoxic red blood cells processed with the Hemanext ONE system and screened during the treatment period. The patient's blood test results are outlined in Table 2. No adverse effects were recorded on Days 1 or 7, and none were reported on Day 14, during the time to the subject's subsequent RBC transfusion. No adverse events were reported for up to 28 days post-transfusion. The patient's pre-transfusion Hb level was 8.1 g/dL and his post-transfusion with hypoxic RBCs Hb level increased to 9.3 g/dL. The patient's pre- and post-transfusion Hct is 25% and his post-transfusion Hct level was increased to 28%.

TABLE 2
Blood Test Results

| | | | Pre- | | | |
|---|---|---|---|---|---|---|
| | Pre-transfusion | | transfusion | | | |
| Parameter | Day 1 | | Day 14 | | Reference value | |
| Hemoglobin | 8.1 | g/dL | 8.4 | g/dL | 13.4-17.0 | g/dL |
| Total white blood cells | 3.1 × 10⁹/L | | 3.1 × 10⁹/L | | 4.1-9.8 × 10⁹/L | |
| Neutrophils | 1.6 × 10⁹/L | | 1.2 × 10⁹/L | | 1.8-6.9 × 10⁹/L | |
| Monocytes | 0.50 × 10⁹/L | | 0.37 × 10⁹/L | | 0.28-0.90 × 10⁹/L | |
| Thrombocytes | 155 × 10⁹/L | | 115 × 10⁹/L | | 145-348 × 10⁹/L | |
| Reticulocytes | 0.036 × 10¹²/L | | 0.027 × 10¹²/L | | 0.030-0.100 × 10¹²/L | |
| Mean corpuscular volume | 101 | fL | 101 | fL | 82-98 | fL |

### Example 4: Hypoxic Red Blood Cell Transfusion for Myelodysplastic Syndromes (MDS) Patients

Patients are enrolled in a study to evaluate the total volume of hypoxic RBCs required for transfusion. The following is recorded during the study:
1. Mean number of transfusion events throughout the study period;
2. Mean change in key laboratory assessments (hemoglobin and hematocrit) and average hemoglobin increment after transfusions of hypoxically stored RBCs compared to that with conventionally stored RBCs;
3. Mean change in QoL as assessed at Day 0, Month 6, and end of study as measured by the EORTC QLQ-C30 and QUALMS (Quality of Life in Myelodysplasia Scale);
4. Mean change from baseline in serum ferritin during the first 12 months;
5. Safety assessment in terms of frequency of adverse event reactions;
6. The number of participants who experienced an adverse event (all types/grades) within a time frame up to 24 hours following the transfusion, up to 7 days (+/−1 day) after the transfusion and between transfusion intervals will be evaluated.

The type and the grade of each adverse event will be categorized according to:
1. Association for the Advancement of Blood and Biotherapies (AABB) technical manual, 20th edition (2020);
2. Delaney et al., “Biomedical Excellence for Safer Transfusion (BEST) Collaborative. Transfusion reactions: prevention, diagnosis, and treatment,” Lancet 388:2825-2836 (2016);
3. Local AEs database (for reference);
4. ISO 14155-2020 (Clinical investigation of medical devices for human subjects—Good Clinical Practice) definitions;

Rate of adverse event reactions and rate of subjects who had a doctor office visit (non-study scheduled), or emergency room visit, or a hospitalization after signing informed consent will be reported.

### Example 5: Safety and Efficacy of Hypoxic Red Blood Cells Processed with the Hemanext ONE System in Patients with Myelodysplastic Syndromes Requiring Chronic Transfusion

The study is a multi-center, randomized, controlled cross-over design comparing the transfusion of hypoxic RBCs in PAGGSM (study group) against the transfusions completed with conventional RBCs.

The following criteria must be met for participation in the study:
1. Male or female aged 18 or older;
2. Have very low, low or intermediate risk MDS per IPSS-R (https://www[dot]mds-foundation[dot]org/ipss-r-calculator/);
3. Bone marrow aspirate completed within the 6 months prior to study enrolment, and which did not show progression to higher risk MDS;
4. Relapse or failure after ESA therapy and/or lenalidomide, azacitidine, luspatercept, or other experimental agents;
5. Have RBC transfusion dependence (at least 2 RBC units/4 weeks during the last 16 weeks);
6. Baseline RBC transfusion threshold of <=8 g/dL;
7. ECOG (Eastern Cooperative Oncology Group) performance status <3;
8. Have signed the informed consent form and are willing to comply with the study visits and procedures; and
9. Have been on a stable dose iron chelation therapy, if started at least 3 months prior to screening.

The following criteria must not be met for participation in the study:
1. Have a life expectancy of less than 1 year;
2. Have palpable splenomegaly (more than 3 cm below the mid clavicular line);
3. Have other associated causes of anaemia (including auto-immune hemolysis or active haemorrhage, or progression to acute leukaemia);
4. Are currently taking erythropoiesis affecting disease modifying agents (i.e., lenalidomide, azacitidine, luspatercept);
5. Have severe renal insufficiency with creatinine clearance (MDRD or CKD EPI) below 30 ml/min;
6. Have lung disease with hypoxia or are oxygen-dependent;
7. Have severe coronary artery disease (including unstable angina or recent myocardial infraction) or severe heart failure (left ventricular ejection fraction less than 30%);
8. Have a history of cancer active in the previous 3 years, except local cervix cancer, or basal cell cutaneous carcinoma;
9. Have a history of allo-immunization other than rhesus Kell;
10. Are a female of child-bearing potential that is pregnant, planning to become pregnant in the next 12 months or breastfeeding;
11. Are a patient under guardianship or curatorship; or
12. Are currently participating in another interventional study.

Participants undergo transfusions with 1 to 3 units of hypoxic Red Blood Cells (i.e., leukocytes reduced, O₂/CO₂ reduced) manufactured with the Hemanext ONE System, or conventional (i.e., non-oxygen reduced blood or non-oxygen and carbon dioxide reduced blood), leukocyte reduced RBCs during the treatment period. Each participant is randomized to treatment with either hypoxic RBCs (Treatment A) or conventional RBCs (Treatment B) after successful completion of the screening period. After 6 months in the randomized treatment arm, two “Wash Out” transfusions of conventional RBCs are scheduled. The participants are then “cross-over” into the other treatment arm to complete another 6 months of treatment.

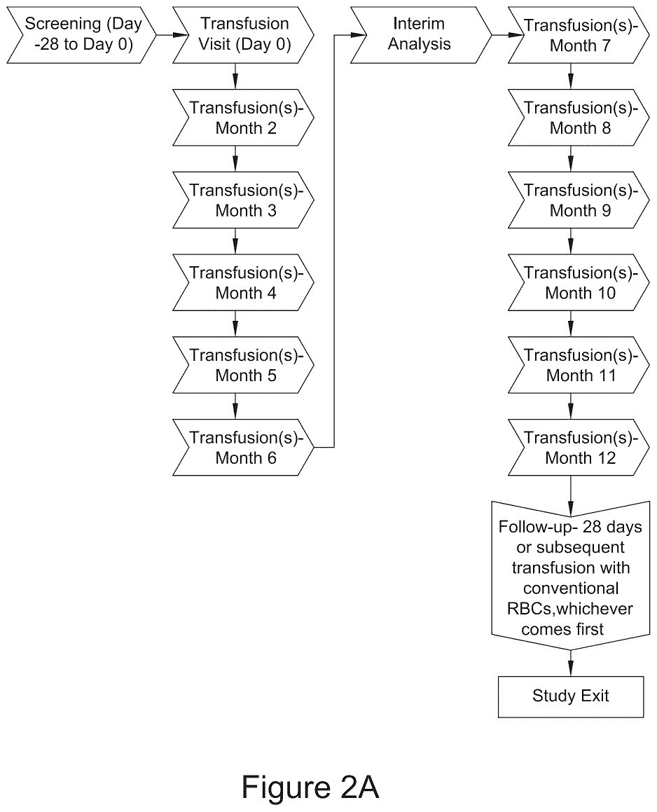

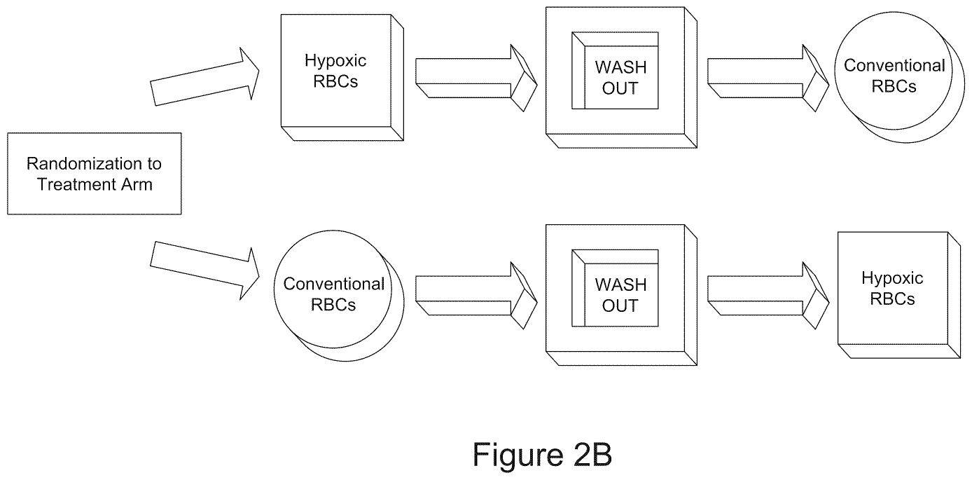

Group 1 (AB) participants receive 6 consecutive months of transfusion exchange procedures with hypoxic RBCs followed by a washout period (2 transfusion cycles), then 6 consecutive months of transfusion exchange procedures with conventional RBCs. Group 2 participants receive 6 consecutive months of transfusion exchange procedures with conventional RBCs followed by a washout period (2 transfusion cycles), then 6 consecutive months of transfusion exchange procedures with hypoxic RBCs, as shown in FIGS. 2A and 2B. The treatment period is a total of 14 months and the total study duration is 26 months, including the 12 month enrollment period.

The following information is collected from enrollment throughout end of study visit, when applicable:
1. Informed consent;
2. Documentation of Inclusion/Exclusion criteria;
3. Demographics;
4. Medical history and concomitant medications/treatments;
5. Routine physical examinations at Day 0 and end of study;
6. Routine laboratory assessments and pregnancy tests (if applicable);
7. Height at the Screening visit;
8. Weight prior to each transfusion;
9. RBC unit weight and volume prior to each transfusion;
10. Age (days) of the RBC unit at the time of each transfusion;
11. Hemoglobin measurements 15 minutes, 60 minutes, and 24 hours after each transfusion;
12. Hemoglobin measurement, at a minimum, 15 minutes before the subsequent transfusion;
13. Blood pressure every 15 minutes during the transfusion and until 15 minutes after the end of the transfusion;
14. Heart rate every 15 minutes during the transfusion and until 15 minutes after the end of the transfusion;
15. Body temperature every 15 minutes during the transfusion and until 15 minutes after the end of the transfusion;
16. Respiratory rate every 15 minutes during the transfusion and until 15 minutes after the end of the transfusion;
17. Oxygen saturation every 15 minutes and until 15 minutes after the end of the transfusion;
18. Serum ferritin measurement at Day 0, Month 3, Month 6, Month 8, Month 11, and end of study; and
19. Patient diary for collection of concomitant medications/treatments and changes in health status from enrollment up to the final study visit. See Table 3.

TABLE 3
Study Visits

| | | Pre- | | Post- | Follow | Follow | |
|---|---|---|---|---|---|---|---|
| | Screening | Transfusion | Transfusion | Transfusion | Up Visit | Up Visit | Study Exit |
| | Visit | | | | | | |
| | Day −28- | Transfusions | | | | | |
| | Day 0 | Month 1 through Month 14 | | | | | Month 14 |
| | Time (hour, day, week) | | | | | | |
| | | | | | | | Month 14 + 1 |
| | | | | | | | (at least 20 |
| | | D 1 | | D 1 | | | days after |
| | | (4 hours to 15 | | (up to 60 | D 2 | D 7 (+/−1 | Month 14 |
| | | minutes prior) | D 1 | minutes after) | (≤24 h) | day) | transfusion) |
| Informed | x | | | | | | |
| Consent | | | | | | | |
| Inclusion/ | x | x | | | | | |
| Exclusion | | | | | | | |
| Randomization | x | | | | | | |
| Transfusion | | | x | | | | |
| with RBCs | | | | | | | |
| Demographics | x | | | | | | |
| Medical History | x | | | | | | |
| Concomitant | x | x | x | x | x | x | x |
| Medications | | | | | | | |
| Physical | x | | | | | | x |
| Examination | | | | | | | |
| Pregnancy | x | x | | | | | |
| Test | | | | | | | |
| Height | x | | | | | | |
| Weight | x | x | | | | | |
| RBC Unit | | x | | | | | |
| Weight | | | | | | | |
| Hemoglobin | | x | | x | x | x | |
| Blood Pressure | x | x | x | x | | | |
| Heart Rate | x | x | x | x | | | |
| Body | x | x | x | x | | | |
| Temperature | | | | | | | |
| Respiratory | x | x | x | x | | | |
| Rate | | | | | | | |
| Oxygen | x | x | x | x | | | |
| Saturation | | | | | | | |
| Laboratory | | x | x | x | | | |
| Measurements | | | | | | | |
| (Standard of | | | | | | | |
| Care) | | | | | | | |
| Serum Ferritin | | x (every 3 | | | | | |
| | | months) | | | | | |
| Patient Diary | | | | x | x | x | x |
| Quality of Life | | x (before first | | x (after Month | | | |
| Questionnaire | | transfusion | | 6 and 14 | | | |
| | | only) | | only) | | | |

Statistical Analysis: Continuous data is summarized by descriptive statistics of n, mean, median, standard deviation, minimum, and maximum. Categorical data is summarized by frequencies and percentages. Hypothesis testing is performed at a two-sided alpha of 0.05.

Baseline comparability is determined by comparing treatment characteristics between the hypoxic RBC transfusion treatments and a historical control from each participant's 12 months of conventional RBC transfusions prior to study enrollment.

The primary efficacy endpoint of the change in volume of RBCs will be analysed using paired t-tests and analysis of variance (ANOVA), where appropriate. Safety data will be summarized with descriptive statistics.

While the present disclosure has been described with reference to particular embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the scope of the present disclosure.

Therefore, it is intended that the present disclosure not be limited to the particular embodiments disclosed as the best mode contemplated for carrying out the present disclosure, but that the present disclosure will include all embodiments falling within the scope and spirit of the appended claims.

### Example 6: Testing Safety of the Administration of Hypoxic Red Blood Cells to a 33-Year-Old Patient with MDS-RS

A 33-year-old female patient with MDS-RS is enrolled in the safety study for the infusion of 2 units of hypoxic red blood cells processed with the Hemanext ONE system. The patient's pre-transfusion Hb level is 8.2 g/dL, and her pre-transfusion Hct is 28%. The patient experienced an AE two days post-transfusion that was due to rhinovirus and deemed unrelated to the study device, the hypoxic blood product and study procedures by the Investigator. The patient's post-transfusion Hb level is increased to 10.6 g/dL and her post-transfusion Hct is increased to 34%. The patient's post-transfusion Hb and Hct levels are higher compared to older patients, e.g. patients in Examples 7, 8, and 9, indicating that age may play a role in incremental Hb increases following blood transfusions.

### Example 7: Testing Safety of the Administration of Hypoxic Red Blood Cells to an Acute Myeloid Leukemia Patient

A 72-year-old male patient with Acute Myeloid Leukemia (AML) is enrolled in the safety study. The patient was transfused with 2 units of hypoxic red blood cells processed with the Hemanext ONE system. The patient's pre-transfusion Hb level is 7.5 g/dL, and his pre-transfusion Hct is 25%. No AEs are reported post-transfusion of hypoxic blood up to the patient's subsequent RBC transfusion (or Day 28). The patient's post-transfusion Hb level is increased to 8.4 g/dL and his post-transfusion Hct is increased to 24%.

### Example 8: Testing Safety of the Administration of Hypoxic Red Blood Cells to a Patient with MDS with Deletion of Part of the Long Arm of Human Chromosome 5q (Del5q)

A 89-year-old male patient with MDS with del5q is enrolled in the safety study. The patient is transfused with 2 units of hypoxic red blood cells processed with the Hemanext ONE system. The patient's pre-transfusion Hb level is 7.8 g/dL, and his pre-transfusion Hct is 23%. The study recorded any occurrence of AEs post-transfusion of hypoxic blood up to the patient's subsequent RBC transfusion (or Day 28). No AEs are reported up to 28 days post-transfusion. The patient's post-transfusion Hb level is increased to 8.8 g/dL and his post-transfusion Hct is increased to 26%.

### Example 9: Testing Safety of the Administration of Hypoxic Red Blood Cells to a Patient with Myelofibrosis

A 75-year-old male patient with Myelofibrosis was enrolled in the safety study for the infusion of 2 units of hypoxic red blood cells. Hypoxic red blood cells are processed with the Hemanext ONE system. The patient's pre-transfusion Hb level is 6.9 g/dL, and his pre-transfusion Hct is 24%. No AEs are reported post-transfusion of hypoxic blood up to the patient's subsequent RBC transfusion (or Day 28). The patient's post-transfusion Hb level is increased to 8.0 g/dL and his post-transfusion Hct is increased to 26%.

Hypoxic RBCs processed with the Hemanext ONE system are well tolerated in patients. Hb levels are within the target range at follow-up, supporting that hypoxically stored RBCs function appropriately. The higher post-transfusion level in a younger, less ill patient supports the concept that age and inflammation play a role in incremental Hb increases following blood transfusions.

### Example 10: Testing Safety of the Administration of Hypoxic Red Blood Cells to Five Patients with Hematological Malignancies

Five patients with haematological malignancies are enrolled and included in a study to test the tolerability of citrate-phosphatedextrose (CPD)/phosphate-adenine-glucose-guanosine-saline-mannitol (PAGGSM) RBCs that are leukocyte-reduced (LR) and O₂/CO₂ reduced, with the aim of improving RBC quality for transfusion. The baseline characteristics of the enrolled patients are shown in Table 4. The mean standard deviation (SD) age of the patients is 69.8±19.3 years, and 80% of the patients are male. Mean body mass index is 24.8±3.4 kg/m². Three patients have a primary diagnosis of MDS, two with ring sideroblasts and one with a del5q mutation. Of the two remaining patients, one has a primary diagnosis of AML, and the other has primary myelofibrosis. The mean time since diagnosis is 6.6±5.3 years. The mean pre-transfusion Hb level is 7.7±0.5 g/dL, and patients are transfused with a mean volume of 478±20.5 mL of 0₂/CO₂ reduced, LR RBCs in PAGGSM having a mean age of 20.8±12.3 days. One adverse event (rhinovirus) is recorded in a single patient within 7 days of the transfusion (primary endpoint), occurring two days post-treatment. The event is mild in severity and is likely unrelated to the treatment. The adverse event is resolved prior to study exit—28 days post-transfusion. No other adverse events occur within 28 days of the transfusion (secondary endpoint).

TABLE 4
Baseline Characteristics
Variable

| | |
|---|---|
| Age, years, mean ± SD | 69.8 ± 19.3 |
| Sex, n (%) | |
| Male | 4 (80) |
| White, n (%) | 5 (100 |
| Body Mass Index (BMI), kg/m2 | 24.8 ± 3.4 |
| Time since diagnosis of hematological malignancy, years | 6.6 ± 5.3 |

The mean Hb level rises from 7.7±0.5 g/dL prior to the transfusion to 9.0±0.9 g/dL post-transfusion, representing an increase of 17%. Patients receive a subsequent transfusion 22.8±6.4 days (mean) after the first transfusion, at which point the mean Hb is 8.2±1.7 g/dL. Blood test values pre-transfusion and at study exit are shown in Table 5.

Although most values remain the same, an increase in the mean total white blood cell count and a reduction in the mean reticulocyte count are observed. The increase in total white blood cells is predominantly based on one patient who has an elevated white blood cell count pre-transfusion. Also, the reduction in reticulocytes predominantly arises from one patient whose pre-transfusion count is well above the reference value; the reticulocyte count of this patient is not determined at the time of study exit.

The hypoxic RBCs processed with the CPD/PAGGSM leukoreduced, oxygen and carbon dioxide reduced system are effective and well tolerated in patients with hematological malignancies. No treatment-related adverse events are reported for up to 28 days after transfusion.

TABLE 5
Blood test parameters before first transfusion and at study exit.

| Parameter | Pre-transfusion | Study Exit | Reference value |
|---|---|---|---|
| Hemoglobin, g/dL | 7.7 ± 0.5 | 8.2 ± 1.7 | 13.4-17.0 |
| Total white blood cells | 25 ± 44 | 35.9 ± 65.6 | 4.1-9.8 |
| (WBC), cells × 10⁹/L | | | |
| Neutrophils, | 1.6 ± 0.7 | 1.1 ± 0.6 | 1.8-6.9 |
| cells × 10⁹/L | | | |
| Monocytes, | 0.5 ± 0.1 | 0.4 ± 0.3 | 0.3-0.9 |
| cells × 10⁹/L | | | |
| Thrombocytes, cells × | 102 ± 69 | 94 ± 74 | 145-348 |
| 10⁹/L | | | |
| Reticulocytes, cells × | 1.61 ± 3.1 | 0.07 ± 0.04 | 0.03-0.10 |
| 10¹²/L | | | |
| Mean corpuscular | 97 ± 8 | 98 ± 8 | 82-98 |
| volume (MCV), fL | | | |

1. A method for reducing the blood transfusion volume for a hematologic malignancy patient in need thereof comprising administering at least two units of oxygen and carbon dioxide reduced, leukoreduced red blood cells to the hematologic malignancy patient in need thereof, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, leukoreduced red blood cells are administered to the hematologic malignancy patient in need thereof in a single transfusion event, and wherein the average volume of subsequent blood transfusions administered to the hematologic malignancy patient in need thereof after the administering of the at least two units of oxygen and carbon dioxide reduced, leukoreduced red blood cells is reduced by at least 10%.

2. (canceled)

3. The method of claim 1, wherein the average volume of subsequent blood transfusions administered to the hematologic malignancy patient in need thereof after the administering of the oxygen and carbon dioxide reduced, leukoreduced red blood cells is reduced by at least 25%.

4. The method of claim 1, wherein the average interval between the subsequent blood transfusions administered to the hematologic malignancy patient in need thereof after the administering of the oxygen and carbon dioxide reduced, leukoreduced red blood cells is increased by at least 10%.

5. (canceled)

6. The method of claim 1, wherein a transfusion-related adverse event does not occur for at least 7 days after the administering of the at least two units of oxygen and carbon dioxide reduced, leukoreduced red blood cells, wherein
a. the transfusion-related adverse event is selected from the group consisting of iron overload, hemorrhagic shock, alloimmunization, an allergic reaction, a transfusion-transmitted infection (TTI), transfusion-associated graft versus host disease, transfusion-related acute lung injury (TRALI), post-transfusion purpura, transfusion-associated circulatory overload (TACO), transfusion-associated dyspnea (TAD), febrile non-hemolytic transfusion reaction (FNHTR), hypotensive transfusion reaction, delayed hemolytic transfusion reaction (DHTR), delayed serologic transfusion reaction (DSTR), acute hemolytic transfusion reaction (AHTR), a major cardiac event (MCE), hematoma, arterial puncture, delayed bleeding, localized infection or inflammation, brachial artery pseudoaneurysm, deep vein thrombosis (DVT), vasovagal reactions, nerve injury or irritation, compartment syndrome, arteriovenous fistula, and any combination thereof; or
b. the transfusion-related adverse event is a serious adverse event selected from the group consisting of a life-threatening illness, a life-threatening injury, permanent impairment of a body structure, permanent impairment of a body function, hospitalization, prolongation of hospitalization, a need for medical or surgical intervention to prevent a life-threatening illness or injury, contraction of a chronic disease, and death.

7. (canceled)

8. (canceled)

9. The method of claim 6, wherein the transfusion-related adverse event does not occur for at least 28 days after the administering.

10. The method of claim 1, wherein the hematologic malignancy is selected from the group consisting of myelodysplastic syndromes (MDS), MDS with ring sideroblasts (MDS-RS), MDS with deletion of part of the long arm of human chromosome 5q (del5q), acute myeloid leukemia (AML), myelofibrosis, myelomatosis, and leukemia, optionally wherein the myelomatosis is multiple myeloma: or optionally wherein the leukemia is acute lymphoblastic leukemia (ALL), chronic lymphocytic leukemia (CLL), acute myelogenous leukemia (AML), or chronic myelogenous leukemia (CML).

11. (canceled)

12. (canceled)

13. The method of claim 1, wherein
a. the hematologic malignancy patient in need thereof has anemia,
b. the hematologic malignancy patient in need thereof requires chronic blood transfusions;
c. the hematologic malignancy patient in need thereof has a hemoglobin level of less than 9 grams per deciliter (g/dL) prior to the administering, optionally wherein the hemoglobin level is increased to 9 g/dL or more after the administering;
d. the hematologic malignancy patient in need thereof has a total white blood cell concentration of less than 4.0×10⁹/L prior to the administering, optionally wherein the total white blood cell concentration is increased to 4.0×10⁹/L or more after the administering;
e. the hematologic malignancy patient in need thereof has a neutrophil concentration of less than 1.8×10⁹/L prior to the administering, optionally wherein the neutrophil concentration is increased to 1.8×10⁹/L or more after the administering;
f. the hematologic malignancy patient in need thereof has a thrombocyte concentration of less than 145×10⁹/L prior to the administering, optionally wherein the thrombocyte concentration is increased after the administering;
g. the hematologic malignancy patient in need thereof has a reticulocyte concentration of less than 0.030×10¹²/L prior to the administering, optionally wherein the reticulocyte concentration is increased after the administering;
h. the hematologic malignancy patient in need thereof has a mean corpuscular volume (MCV) of blood that is greater than 98 femtoliters (fL) prior to the administering, optionally wherein the MCV is reduced to 98 fL or less after the administering; or
i. any combination of a to h.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. The method of claim 1, wherein the administering is over a period of about two hours.

29. The method of claim 1, wherein the storage period is less than 2 days, less than 7 days, less than 11 days, less than 21 days, less than 29 days, less than 35 days, less than 42 days-er-4-days.

30. The method of claim 1, wherein the at least two units of oxygen and carbon dioxide reduced, leukoreduced red blood cells further comprise a mixture of citrate-phosphate-dextrose (CPD) and phosphate-adenosine-guanosine-glucose-saline-mannitol (PAGG-SM).

31. The method of claim 1, wherein
a. the SO₂ is 10% or less,
b. the pCO₂ is 15 mmHg or less: or
c. both a and b.

32. (canceled)

33. A method for increasing the hemoglobin increment of a hematologic malignancy patient in need thereof comprising administering at least two units of oxygen and carbon dioxide reduced, leukoreduced red blood cells to the hematologic malignancy patient in need thereof, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, leukoreduced red blood cells are administered to the hematologic malignancy patient in need thereof in a single transfusion event, and wherein the hemoglobin increment of the hematologic malignancy patient in need thereof is increased by at least 1.0 grams per deciliter (g/dL) after the administering.

34. The method of claim 33, wherein iron overload is reduced in the hematologic malignancy patient in need thereof after the administering.

35. A method for improving oxygen delivery to a hematologic malignancy patient in need thereof comprising administering at least two units of oxygen and carbon dioxide reduced, leukoreduced red blood cells to the hematologic malignancy patient in need thereof, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, leukoreduced red blood cells are administered to the hematologic malignancy patient in need thereof in a single transfusion event, and wherein the blood oxygen level of the hematologic malignancy patient in need thereof is increased by at least 10% after the administering.

36. The method of claim 35, wherein the blood oxygen level of the hematologic malignancy patient in need thereof is increased by at least 25% after the administering.

37. The method of claim 35, wherein
the blood oxygen level of the hematologic malignancy patient in need thereof is less than 90% prior to the administering; or
the blood oxygen level of the hematologic malignancy patient in need thereof is at least 95% prior to the administering.

38. (canceled)

39. A method for preventing or reducing transfusion-related sequelae in a hematologic malignancy patient in need thereof comprising administering at least two units of oxygen and carbon dioxide reduced, leukoreduced red blood cells to the hematologic malignancy patient in need thereof, the oxygen and carbon dioxide reduced, leukoreduced red blood cells having an initial oxygen saturation (SO₂) of 20% or less and an initial partial pressure of carbon dioxide (pCO₂) of 30 millimeters of mercury (mmHg) or less and maintained at an oxygen saturation of 20% or less and a pCO₂ of 30 mmHg or less for a storage period, wherein the hematologic malignancy patient in need thereof is a blood transfusion dependent patient, wherein the at least two units of oxygen and carbon dioxide reduced, leukoreduced red blood cells are administered to the hematologic malignancy patient in need thereof in a single transfusion event, and wherein the occurrence of transfusion-related sequelae is reduced over a 7 day period after the administering compared to a hematologic malignancy patient in need thereof administered at least two units of conventional red blood cells.

40. The method of claim 39, wherein less than two transfusion-related sequelae occur over the 7 day period.

41. The method of claim 39, wherein the occurrence of transfusion-related sequelae is reduced over a 28 day period after the administering compared to a hematologic malignancy patient in need thereof administered at least two units of conventional red blood cells, optionally wherein less than two transfusion-related sequelae occur over the 28 day period.

42. (canceled)

43. The method of any one of claim 39, wherein the transfusion-related sequelae are selected from the group consisting of dyspnea, fever, chills, facial flushing, severe pain, shock, rapid pulse, low blood pressure, hypothermia, respiratory distress, nausea, vomiting, itching, and jaundice.

44. (canceled)

45. (canceled)

46. (canceled)

47. (canceled)

48. (canceled)
